(19) United States
(12) Patent Application Publication
(10) Pub. No.: US 2025/0043300 A1
(43) Pub. Date: Feb. 6, 2025

(54) METHODS TO INCREASE YIELDS IN CROPS

(71) Applicant: THE UNIVERSITY OF WARWICK, Warwickshire (GB)

(72) Inventor: Jose F. GUTIERREZ-MARCOS, Warwickshire (GB)

(73) Assignee: THE UNIVERSITY OF WARWICK, Warwickshire (GB)

(21) Appl. No.: 18/715,201

(22) PCT Filed: Dec. 13, 2022
(86) PCT No.: PCT/GB2022/053196

§ 371 Date: May 31, 2024

(30) Foreign Application Priority Data
Dec. 14, 2021 (GB) 2118058.3

(51) Int. Cl. C12N 15/82; C07K 14/415

(52) U.S. Cl. CPC C12N 15/8261; C07K 14/415

(57) ABSTRACT

The present invention relates to a method of producing a modified plant, part, cell or protoplast thereof having an increase in a yield-related trait relative to a reference plant, part, cell or protoplast thereof. More particularly, the invention relates to providing the plant, part, cell or protoplast thereof with an RKD transcription factor, a polynucleotide encoding the same, or a gene editing system for modifying an endogenous nucleic acid sequence encoding an RKD transcription factor or regulatory sequence thereof. Said plants have improvements in a yield-related trait, and thereby improved yield, which is especially useful in agriculture.

### FIELD OF THE INVENTION

The present invention relates to a method of producing a modified plant, part, cell or protoplast thereof having an increase in a yield-related trait relative to a reference plant, part, cell or protoplast thereof. More particularly, the invention relates to providing the plant, part cell or protoplast thereof with RKD transcription factor, a polynucleotide encoding the same or a gene editing system for modifying an endogenous nucleic acid sequence encoding an RKD transcription factor or regulatory sequence thereof. The invention also relates to modified plant, part, cell or protoplast thereof obtained by the methods. Said plants have improvements in a yield-relate trait.

### BACKGROUND OF THE INVENTION

Meeting the demands of global food production is an ever-increasing challenge, with the human population expected to exceed 9 billion by 2050 (World Population Prospects, 2019). The increasing human population coupled with unpredictable weather events driven by climate change have resulted in a threat to food security worldwide. To feed the growing population, it is estimated that food crop production will have to be increased by approximately 25-70% above current production levels (Hunter, et al., 2017). However, expansion of arable land through deforestation has significant drawbacks including increased soil erosion, the loss of habitats and declining ecological biodiversity.

The majority of modern day crops have been domesticated from their wild progenitors. Selective breeding strategies have been used to introduce preferable traits into plant architecture. Examples include the domestication of wheat resulting from hybridisation events over the past ten thousand years. Around ˜10-12,000 years ago, diploidy wild wheat was introgressed into the tetraploid wheat and landrace wheat was established. Modern bread wheat, Triticum aestivum, is an allohexaploid (AABBDD) that was domesticated between late 19th and early 20th century via plant breeding to select for more upright plants. In the case of rice, upright architecture has been established and spikelet branches and effective tillers have been increased. In maize, tiller branches have been decreased and cob size and number of seeds have been increased. In aspects of wheat and barley, spike lengths and branch numbers have been increased (Alseekh et al., 2021).

Several traits in crops are determined by meristem specification. The meristem is a type of tissue found in plants, that consists of undifferentiated cells capable of cell division. Cells in the meristem can develop into all other tissues and organs that occur in plants. These cells continue to divide until a time when they become differentiated. Meristems can determine several important yield related traits of crop species. Maize inflorescence meristems determine seed numbers of cobs and branch number of tassels. Wheat floral meristems can control floral branch number. Rice shoot apical meristem and axillary meristem are involved in tiller number.

Grasses are a family of monocotyledonous flowering plants with ˜12,000 species. Grasses account for 70% of the crops grown worldwide and contribute roughly the same amount to calorific intake through direct consumption and animal feed. Grass plants have a highly differentiated embryo structure and can be broken into two major clades; the BOP clade and PACMAD clade. Both clades comprise economically important crops including wheat, oats and rye in the BOP clade and maize, millet and sorghum in the PACMAD clade.

Tillering in grasses is an important agronomic trait for yield and grain production. This agronomic trait has been subject to artificial selection during the process of domestication of wild ancestors as discussed above. Tillers are formed by the initiation and outgrowth of axillary meristems that determine the number of grain-bearing branches formed. Differences in axillary meristems formation and development produces the diversity of plant architecture seen amongst the grasses. The molecular mechanisms underpinning meristem formation in dicots have been well characterised through the study of mutants with defects in shoot development. Most mutants exhibiting increased meristem formation have been found to be due to factors involved in the regulation of apical dominance and branching by phytohormones. However, the identification of key factors controlling plant architecture and seed yield has been more challenging in economically significant polyploid grasses such as wheat.

Increasing yield related traits and grain production in food crops by selective breeding is slow and lacks control over undesirable traits being introduced into the offspring through random mutation events. One solution to increase crop yields without expanding arable land or using slow selective breeding techniques is to identify the mechanisms underpinning plant architecture and either select for these mechanisms, or artificially enhance such mechanisms in the crop. However, as discussed, identifying the mechanisms underpinning plant architecture in polypoid grasses is challenging.

The inventors of the present invention have made the surprising discovery that a group of transcription factors named the ‘RKD’ transcription factors, a subfamily of plant-specific RWP-RK transcription factors, control the formation of axillary meristems which determine several important traits of crop species. Previous studies have shown that RKD factors are evolutionary conserved regulators of germ cell differentiation in land plants (Koi et al., 2016). However, thus far, these studies have only shown RKD factors to have a role in embryonic development.

The present invention is based on the discovery that these RKD transcription factors can also control the formation of axillary meristems in vegetative tissues of plants. The inventors have shown that ectopic expression of such transcription factors results in a significant increase in tiller number and seed yield in several important crop plant species. The inventors have therefore discovered a unique strategy to increase crop yields to help improve global food security.

### SUMMARY OF THE INVENTION

According to a first aspect of the invention there is provided a method of producing a modified plant, plant part, cell or protoplast thereof having an increase in a yield-related trait relative to a reference plant, plant part, cell or protoplast thereof, comprising:
(a) Providing the plant, plant part, cell or protoplast thereof with an RKD transcription factor or a variant or active fragment thereof, or a polynucleotide encoding said RKD transcription factor, variant or active fragment thereof; or
(b) Providing the plant, plant part, cell or protoplast thereof with a gene editing system for modifying an endogenous nucleic acid sequence encoding an RKD transcription factor or a regulatory sequence controlling expression thereof;
(c) Obtaining a modified plant, plant part, cell or protoplast thereof having an increase in expression of the RKD transcription factor; and
(d) Optionally regenerating a modified plant from the or each plant part, cell or protoplast.

According to a second aspect, there is provided a method of increasing a yield-related trait in a plant, plant part, cell or protoplast thereof relative to a reference plant, plant part, cell or protoplast thereof comprising increasing the expression of an RKD transcription factor in the plant, plant part, cell or protoplast thereof.

In one embodiment of the second aspect, the method comprises the steps as defined in the first aspect. Therefore in one embodiment there is provided a method of increasing a yield-related trait in a plant, plant part, cell or protoplast thereof relative to a reference plant, plant part, cell or protoplast thereof comprising increasing the expression of an RKD transcription factor in the plant, part, cell or protoplast thereof by:
(a) Providing the plant, plant part, cell or protoplast thereof with an RKD transcription factor or a variant or active fragment thereof, or a polynucleotide encoding said RKD transcription factor, variant or active fragment thereof; or
(b) Providing the plant, plant part, cell or protoplast thereof with a gene editing system for modifying an endogenous nucleic acid sequence encoding an RKD transcription factor or a regulatory sequence controlling expression thereof;
(c) Obtaining a modified plant, plant part, cell or protoplast thereof having an increase in RKD transcription factor expression; and
(d) Optionally regenerating a modified plant from the or each plant part, cell or protoplast.

In a third aspect, there is provided a modified plant, plant part, cell or protoplast thereof obtained by the first or second aspects.

Suitably, in one embodiment, the modified plant, plant part, cell or protoplast thereof comprises an increase in a yield-related trait relative to a reference plant, plant part, cell or protoplast

In a fourth aspect, there is provided a seed, other plant part or material derived from the modified plant of the first, second or third aspect.

According to a fifth aspect, there is provided use of an RKD transcription factor or a variant or active fragment thereof, or a polynucleotide encoding said RKD transcription factor or a variant or active fragment thereof, in the production of a plant, plant part, cell or protoplast thereof which has an increase in a yield-related trait relative to a reference plant, plant part, cell or protoplast thereof.

In a sixth aspect as provided herein, there is provided use of an RKD transcription factor or a variant or active fragment thereof, or a polynucleotide encoding said RKD transcription factor or a variant or active fragment thereof, or an associated marker thereof, as a selectable marker in plant transformation.

In one embodiment, the marker is used for selecting plants having an increase in a yield-related trait.

According to a seventh aspect of the present invention, there is provided a method of selecting a plant comprising an increase in a yield-related trait relative to a reference plant, comprising:
(a) Providing a plant;
(b) Optionally mutagenizing the plant;
(c) Measuring the expression level of at least one RKD transcription factor or an associated marker thereof in at least one cell of the plant;
(d) Comparing the measured expression level to a reference expression level of the same RKD transcription factor or an associated marker thereof in a reference plant; and
(e) Selecting the plant if the measured expression level is increased relative to the reference expression level.

In one embodiment, the plant may have been produced or obtained by the method of the first or second aspects, or may be a plant of the third aspect.

In an eighth aspect of the present invention, there is provided a method of plant breeding or plant improvement comprising combining the genetic material of a first plant obtained by the method of the first or second aspects with the genetic material of a second plant.

In one embodiment, the second plant may be a plant obtained by the method of the first or second aspect, or according to the third aspect, or may be another plant differing in genotype from the first plant.

In one embodiment, progeny are produced which comprise increased expression of an RKD transcription factor thereof relative to a reference plant.

In one embodiment, the method is a method of breeding plants or improving plants such that they have an increase in a yield-related trait relative to a reference plant.

According to a ninth aspect, there is provided a method of producing a hybrid seed comprising crossing a first modified plant obtained by the method of the first or second aspects, or according to the third aspect, with a second plant; and obtaining a seed.

In one embodiment, the second plant may be a plant obtained by the method of the first or second aspects, or according to the third aspect, or may be another plant differing in genotype from the first plant.

In one embodiment, the seed comprises a genotype which confers an increased expression of an RKD transcription factor thereof relative to a reference plant.

According to a tenth aspect, there is provided a hybrid seed obtained by the ninth aspect.

According to an eleventh aspect, there is provided a hybrid plant generated from the hybrid seed of the tenth aspect.

### DETAILED DESCRIPTION OF EMBODIMENTS OF THE INVENTION

Certain features and embodiments of the aspects above will now be described under the following headed sections, any feature or embodiment under any section may be combined with an applicable aspect of the invention.

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

### RKD Transcription Factor

Surprisingly, the inventors have found that RKD transcription factors, a subfamily of plant-specific RWP-RK transcription factors, control yield-related traits in plants by controlling axillary meristem formation. The inventors have found that plants which are modified to increase expression of said RKD transcription factors have increases in yield-related traits.

Suitably the RKD transcription factor is any RKD transcription factor derived from any plant. Suitably the RKD transcription factor is an RKD transcription factor derived from a crop plant.

In some embodiments, the RKD transcription factor is vegetative or reproductive. In a preferred embodiment the RKD transcription factor is a reproductive RKD transcription factor.

Suitably reproductive RKD transcription factor is an RKD transcription factor that is predominantly expressed in the reproductive tissue of a wild type plant, suitably in the embryonic tissue of a wild type plant. In some embodiments, a reproductive RKD transcription factor is expressed only in the reproductive tissues of a wild type plant. In some embodiments, a reproductive RKD transcription factor may be considered to be an embryonic RKD transcription factor.

Suitably a vegetative RKD transcription factor is an RKD transcription factor that is predominantly expressed in the vegetative tissues of a wild type plant, suitably in the non-embryonic tissues of a wild type plant. In some embodiments, a vegetative RKD transcription factor is expressed only in the vegetative tissues of a wild type plant.

Suitably therefore in a preferred embodiment, the RKD transcription factor is an endogenous reproductive RKD transcription factor.

In some embodiments, the RKD transcription factor or a variant or active fragment thereof may comprise or consist of a sequence according to any one of SEQ ID NO: 1, 4, 6-37 or a functional variant thereof or a sequence having high identity thereto.

By high identity thereto it is meant a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a reference sequence.

Functional variants of a RKD transcription factor sequence may include sequences which vary from the reference sequences, but which substantially retain activity as RKD transcription factor. It will be appreciated by the skilled person that it is possible to vary the sequence of the RKD transcription factor while retaining its ability to regulate transcription. A functional variant of a RKD transcription factor can comprise substitutions, deletions and/or insertions compared to a reference sequence, provided they do not render the RKD transcription factor substantially non-functional. Suitably, a functional variant of a RKD transcription factor may increase chromatin-related factors, increase histone modification factors, increase cell cycling factors, increase DNA methylation factors, increase histone H4 and/or increase H3K27 methylation in the modified plant, plant part, cell or protoplast thereof having an increase in a yield-related trait.

In some embodiments, a functional variant of a RKD transcription factor can be viewed as a RKD transcription factor which, when substituted in place of a reference RKD transcription factor, substantially retains its activity. For example, a functional variant of a given a RKD transcription factor preferably retains at least 80% of its activity, more preferably at least 90% of its activity, more preferably at least 95% of its activity, and yet more preferably 100% of its activity (compared to the reference RKD transcription factor).

Suitably, functional variants of a RKD transcription factor retain a significant level of sequence identity to a reference RKD transcription factor. Suitably functional variants comprise a sequence that is at least 70% identical to the reference RKD transcription factor, more preferably at least 80%, 90%, 95%, 98% or 99% identical to the reference RKD transcription factor.

Suitably the amino acid sequences of the RKD transcription factors referred to herein are present at the end of this document. Suitably any of the sequences referred to may encompass a functional variant thereof or a sequence having high identity thereto.

In some embodiments, the RKD transcription factor is selected from the group consisting of RKD1, RKD2, RKD4, RKD5, RKD6, RKD6a, RKD6b, RKD7, RKD8, RKD9, RKD10, and RKD11, suitably derived from any plant, or a functional variant thereof, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, the RKD transcription factor is selected from the group consisting of OsRKD1, OsRKD3, OsRKD4, OsRKD5, OsRKD6, OsRKD7, OsRKD8, OsRKD9, OsRKD10, TaRKD6a, TaRKD6b, TaRKD9, TaRKD11, AetRKD6a, AetRKD6b, TaRKD1, TaRKD3, TaRKD4, TaRKD10, AetRKD1, AetRKD3 and AetRKD4, or a functional variant thereof, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity thereto. References to ‘TaRKD1’ as used herein may be used interchangeably with ‘TaRKD2’. TaRKD2 is the old name for TaRKD1, therefore either of these names may be considered to be a reference to the same transcription factor.

As described above, the RKD transcription factor may be vegetative. A vegetative RKD transcription factor may be selected from the group consisting of RKD6a, RKD6b, RKD9, and RKD11, suitably derived from any plant. In one embodiment, a vegetative RKD transcription factor may be selected from: TaRKD6a, TaRKD6b, TaRKD9, TaRKD11, AetRKD6a, AetRKD6b and AetRKD9.

Suitably a TaRKD6a RKD transcription factor may be TaRKD6a-2a, TaRKD6a-2b, or TaRKD6a-2d v1 or v2.

Suitably a TaRKD6b transcription factor may be TaRKD6b-2a, or TaRKD6b-2b.

Suitably a TaRKD9 transcription factor may be TaRKD9-3a, TaRKD9-3b, or TaRKD9-3d. Suitably a TaRKD11 transcription factor may be TaRKD11-7a, TaRKD11-7b or TaRKD11-7d.

Suitably a AetRKD6a transcription factor may be AetRKD6a-2d v1 or v2.

Suitably a AetRKD6b transcription factor may be AetRKD6b-2d.

Suitably a AetRKD9 transcription factor may be AetRKD9-3d.

More particularly, a vegetative RKD transcription factor may be selected from the group consisting of: TaRKD6a-2a, TaRKD6a-2b, TaRKD6a-2d v1; TaRKD6a-2d v2; TaRKD6b-2a, TaRKD6b-2b; TaRKD9-3a, TaRKD9-3b, TaRKD9-3d; TaRKD11-7a; AetRKD6a-2d;

AetRKD6b-2d and AetRKD9-3d.

As described above, the RKD transcription factor may be reproductive. A reproductive RKD transcription factor may be selected from the group consisting of RKD1, RKD2, RKD3, RKD4, and RKD10, suitably derived from any plant. A reproductive RKD transcription factor may be selected from the group consisting of: TaRKD1, TaRKD3, TaRKD4, TaRKD10, AetRKD1, AetRKD3, and AetRKD4.

Suitably a TaRKD1 transcription factor may be TaRKD1-7a, TaRKD1-7b, or TaRKD1-7d.

Suitably a TaRKD3 transcription factor may be TaRKD3-7a, TaRKD3-7b, or TaRKD3-7d.

Suitably a TaRKD4 transcription factor may be TaRKD4-6a, TaRKD4-6b, or TaRKD4-6d.

Suitably a TaRKD10 transcription factor may be TaRKD10-7a, TaRKD10-7b, TaRKD10-7d.

Suitably a AetRKD1 transcription factor may be AetRKD1-7d.

Suitably a AetRKD3 transcription factor may be AetRKD3-7d.

Suitably a AetRKD4 transcription factor may be AetRKD4-7d.

More particularly, a reproductive RKD transcription factor may be selected from the group consisting of: TaRKD1-7a, TaRKD1-7b, TaRKD1-7d; TaRKD3-7a, TaRKD3-7d; TaRKD4-6a, TaRKD4-6b, TaRKD4-6d; TaRKD10-7a, TaRKD10-7d; AetRKD1-7d; AetRKD3-7d and AetRKD4-7d.

In some embodiments, the RKD transcription factor may comprise the motif RWPHRK (SEQ ID NO: 2). In some embodiments, the RKD transcription factor be any plant-specific RWP-RK transcription factor that comprises the motif RWPHRK (SEQ ID NO: 2).

Suitably, in such an embodiment, the RKD transcription factor may be selected from the group consisting of: TaRKD6a, TaRKD6b, TaRKD11, TaRKD1, TaRKD3, TaRKD4, TaRKD10, AetRKD1, AetRKD3, AetRKD4, AetRKD6a, AetRKD6b, OsRKD3, OsRKD4, OsRKD5, OsRKD6, and OsRKD8, or any of the specific forms of these transcription factors listed above.

In some embodiments, the RKD transcription factor may comprise the motif KSSGGSRPAAGGKSLDHIGFEELRTYFYMPITKAAREMNVGLTVLKKRCRELGVARWPHR KMKSLRSLILNIQDMGKGATSPAAVQG (SEQ ID NO: 3). In some embodiments, the RKD transcription factor may be any plant-specific RWP-RK transcription factor that comprises the motif: KSSGGSRPAAGGKSLDHIGFEELRTYFYMPITKAAREMNVGLTVLKKRCRELGVARWPHR KMKSLRSLILNIQDMGKGATSPAAVQG (SEQ ID NO: 3). Suitably in such an embodiment, the RKD transcription factor is an RKD1 transcription factor, suitably a TaRKD1 transcription factor. Suitably, in such an embodiment, the RKD transcription factor may be selected from the group consisting of: TaRKD1-7A (SEQ ID NO:1) or a functional variant thereof or a sequence having high identity thereto.

In some embodiments, the RKD transcription factor may comprise the motif KSSGGARPAAGGKSLDHIGFEELRTYFYMPITKAAREMNVGLTVLKKRCRELGVARWPHR KMKSLRSLILNIQEMGKGATSPAAVQG (SEQ ID NO: 5). In some embodiments, the RKD transcription factor may be any plant-specific RWP-RK transcription factor that comprises the motif: KSSGGARPAAGGKSLDHIGFEELRTYFYMPITKAAREMNVGLTVLKKRCRELGVARWPHR KMKSLRSLILNIQEMGKGATSPAAVQG (SEQ ID NO: 5). Suitably, in such an embodiment, the RKD transcription factor is an RKD1 transcription factor, suitably a TaRKD1 transcription factor. Suitably, in such an embodiment, the RKD transcription factor may be selected from the group consisting of: TaRKD1-7b (SEQ ID NO: 4), TaRKD1-7d (SEQ ID NO: 6) and AetRKD1-7d (SEQ ID NO: 24) or a functional variant thereof or a sequence having high identity thereto.

In some embodiments, the RKD transcription factor is a wheat RKD transcription factor. Suitably, the RKD transcription factor is a Triticum aestivum RKD factor, preferably TaRKD1, or TaRKD4. In some such embodiments, the TaRKD1 factor is selected from: TaRKD1-7d (SEQ ID NO: 6), TaRKD1-7a (SEQ ID NO: 1) and TaRKD1-7b (SEQ ID NO: 4) or a functional variant thereof or a sequence having high identity thereto. In one such embodiment, the RKD transcription factor is TaRKD1-7d (SEQ ID NO: 6) or a functional variant thereof or a sequence having high identity thereto.

In some embodiments, the RKD transcription factor is a rice RKD transcription factor. Suitably, the RKD transcription factor is an Oryza sativa RKD factor, preferably OsRKD3 (SEQ ID NO:31), OsRKD4 (SEQ ID NO:32), OsRKD5 (SEQ ID NO:33), OsRKD6 (SEQ ID NO: 34) or OsRKD8 (SQ ID NO: 36) or a functional variant thereof or a sequence having high identity thereto. In one embodiment, the RKD transcription factor is OsRKD3 (SEQ ID NO: 31) or a functional variant thereof or a sequence having high identity thereto.

In some embodiments, the RKD transcription factor is an Arabidopsis thaliana RKD factor. In one embodiment, the RKD transcription factor is AtRKD4 (AT5G53040; SEQ ID NO: 39) or a functional variant thereof or a sequence having high identity thereto.

In some embodiments, the RKD transcription factor is a wild wheat transcription factor. Suitably the RKD transcription factor is an Aegilops tauschii RKD factor, preferably AetRKD1-7D (SEQ ID NO:24), AetRKD3-7D (SEQ ID NO:25), AetRKD4-7D (SEQ ID NO: 26), AetRKD6a-2D (SEQ ID NO:27), AetRKD6b-2D (SEQ ID NO:28), or AetRKD9-3D (SEQ ID NO:29) or a functional variant thereof or a sequence having high identity thereto.

Suitably the RKD transcription factor may be derived from the same species of plant as the modified plant, i.e. homologous, or may be derived from a different species of plant to the modified plant, i.e. heterologous. The RKD transcription factor may be heterologous to the plant or may be homologous to the plant. Preferably, the RKD transcription factor is homologous to the plant. In one embodiment, the RKD transcription factor is a homologous RKD transcription factor. Suitably, for example, wheat plants are provided with or modified with a wheat RKD transcription factor, or a polynucleotide encoding a wheat RKD transcription factor. Suitably, for example, rice plants are provided with or modified with a rice RKD transcription factor or polynucleotide encoding a rice RKD transcription factor.

However, suitably the RKD transcription factor, or a polynucleotide encoding the RKD transcription factor is exogenous to the plant. Suitably therefore the RKD transcription factor, or a polynucleotide encoding the RKD transcription factor is introduced into the plant from outside, and originated outside the plant.

In one preferred embodiment of the method of the invention, there is provided a method of producing a modified wheat (Triticum Aestivum) plant, plant part, cell or protoplast thereof having an increase in a yield-related trait relative to a reference wheat plant, plant part, cell or protoplast thereof, comprising:
(a) Providing the wheat plant, plant part, cell or protoplast thereof with an RKD transcription factor selected from TaRKD1-7d (SEQ ID NO: 6), TaRKD1-7a (SEQ ID NO: 1) and TaRKD1-7b (SEQ ID NO: 4), or a variant or active fragment thereof, or a polynucleotide encoding said RKD transcription factor, variant or active fragment thereof;
(b) Obtaining a modified plant, plant part, cell or protoplast thereof having an increase in expression of the RKD transcription factor; and
(c) Optionally regenerating a modified plant from the or each plant part, cell or protoplast.

Suitably providing comprises introducing the RKD transcription factor selected from TaRKD1-7d (SEQ ID NO: 6), TaRKD1-7a (SEQ ID NO: 1) andTaRKD1-7b (SEQ ID NO: 4), or a variant or active fragment thereof, or a polynucleotide encoding said RKD transcription factor, variant or active fragment thereof into the plant, plant part, cell or protoplast thereof. Suitably providing comprises modifying the wheat plant, suitably transforming or transducing the wheat plant. Suitably modifying the wheat plant to have an increased expression of the RKD transcription factor.

In one preferred embodiment of the method of the invention, there is provided a method of producing a modified rice (Oryza sativa) plant, plant part, cell or protoplast thereof having an increase in a yield-related trait relative to a reference rice plant, plant part, cell or protoplast thereof, comprising:
(a) Providing the rice plant, plant part, cell or protoplast thereof with an RKD transcription factor selected from OsRKD3 (SEQ ID NO:31), or a variant or active fragment thereof, or a polynucleotide encoding said RKD transcription factor, variant or active fragment thereof;
(b) Obtaining a modified plant, plant part, cell or protoplast thereof having an increase in expression of the RKD transcription factor; and
(c) Optionally regenerating a modified plant from the or each plant part, cell or protoplast.

Suitably providing comprises introducing the RKD transcription factor selected from OsRKD3 (SEQ ID NO:31), or a variant or active fragment thereof, or a polynucleotide encoding said RKD transcription factor, variant or active fragment thereof into the plant, plant part, cell or protoplast thereof. Suitably providing comprises modifying the rice plant, suitably transforming or transducing the rice plant. Suitably modifying the rice plant to have an increased expression of the RKD transcription factor.

Suitably, the RKD transcription factor or a variant or active fragment thereof, or a polynucleotide encoding said RKD transcription factor is exogenous.

Suitably the RKD transcription factor or a variant or active fragment thereof, or a polynucleotide encoding said RKD transcription factor is introduced into the plant. Suitable means of introducing such molecules are discussed elsewhere herein.

Suitably the method may be a method of increasing a yield-related trait in a wheat plant, plant part, cell or protoplast thereof relative to a reference wheat plant, plant part, cell or protoplast thereof comprising increasing the expression of an RKD transcription factor in the wheat plant, plant part, cell or protoplast thereof, wherein the method comprises the steps above.

### Yield-Related Trait

The term ‘Yield-related trait’ as used herein may refer to any trait which affects the yield of the plant. Suitably, in some embodiments a yield-related trait may refer to one or more of a plurality of traits which affect the yield of the plant.

Yield may be measured in a number of ways and may refer to vegetative or reproductive parts of the plant, depending on the crop and whether biomass, leaf number, harvest index or seed number or seed size is desired.

In preferred embodiments, a yield-related trait is selected from: (i) number of axillary meristems; (ii) number of tillers; (iii) rachis length; (iv) spike length; (v) number of spikelets per spike; (vi) number of grains per spike; (vii) panicle branch number; (viii) seed weight and/or number of seeds; and/or (ix) thousand kernel weight.

Suitably, a yield-related trait may be a combination of more than one of the listed traits.

Suitably, an increase in a yield-related trait is selected from: (i) an increase in the number of axillary meristems; (ii) an increase in the number of tillers; (iii) an increase in the rachis length; (iv) an increase in the spike length; (v) an increase in the number of spikelets per spike; (vi) an increase in the number of grains per spike; (vii) an increase in the panicle branch number; (viii) an increase in the seed weight and/or an increase in the number of seeds; and/or (ix) an increase in the thousand kernel weight relative to a reference plant.

Suitably, an increase in a yield-related trait may be an increase in a combination of more than one of the listed traits. Suitably therefore an increase in a yield-related trait may be an increase in one or more than one of the listed traits relative to a reference plant, or may be a combined increase in more than one yield-related trait relative to the combined traits in a reference plant.

In some embodiments, the yield-related trait is number of axillary meristems and/or number of tillers. Suitably, an increase in yield-related trait may be an increase in number of axillary meristems and/or an increase in the number of tillers relative to a reference plant. Suitably in such embodiments, the plant is wheat.

In some embodiments, the yield-related trait is panicle branch number and/or number of seeds per panicle. Suitably, an increase in yield-related trait may be increased panicle branch number and/or increased number of seeds per panicle relative to a reference plant. Suitably in such embodiments that plant is rice.

In some embodiments, the yield-related trait may be size, weight and/or number of seeds or grain. Suitably, an increase in yield-related trait may be an increase in size, weight and/or number of seeds or grain relative to a reference plant. Suitably in such embodiments the plant is wheat or rice.

In one preferred embodiment of the method of the invention, there is provided a method of producing a modified wheat (Triticum Aestivum) plant, plant part, cell or protoplast thereof having an increase in number of axillary meristems and/or number of tillers relative to a reference wheat plant, plant part, cell or protoplast thereof, comprising:
(a) Providing the wheat plant, plant part, cell or protoplast thereof with an RKD transcription factor selected from TaRKD1-7d (SEQ ID NO: 6), TaRKD1-7a (SEQ ID NO: 1) and TaRKD1-7b (SEQ ID NO: 4), or a variant or active fragment thereof, or a polynucleotide encoding said RKD transcription factor, variant or active fragment thereof;
(b) Obtaining a modified plant, plant part, cell or protoplast thereof having an increase in expression of the RKD transcription factor; and
(c) Optionally regenerating a modified plant from the or each plant part, cell or protoplast.

In one preferred embodiment of the method of the invention, there is provided a method of producing a modified rice (Oryza sativa) plant, plant part, cell or protoplast thereof having an increase in panicle branch number and/or number of seeds per panicle relative to a reference rice plant, plant part, cell or protoplast thereof, comprising:
(a) Providing the rice plant, plant part, cell or protoplast thereof with an RKD transcription factor selected from OsRKD3 (SEQ ID NO:31), or a variant or active fragment thereof, or a polynucleotide encoding said RKD transcription factor, variant or active fragment thereof;
(b) Obtaining a modified plant, plant part, cell or protoplast thereof having an increase in expression of the RKD transcription factor; and
(c) Optionally regenerating a modified plant from the or each plant part, cell or protoplast.

Suitably, the RKD transcription factor or a variant or active fragment thereof, or a polynucleotide encoding said RKD transcription factor is exogenous.

Suitably the RKD transcription factor or a variant or active fragment thereof, or a polynucleotide encoding said RKD transcription factor is introduced into the plant. Suitable means of introducing such molecules are discussed elsewhere herein.

Suitably the method may be a method of increasing number of axillary meristems and/or number of tillers in a wheat plant, plant part, cell or protoplast thereof relative to a reference wheat plant, plant part, cell or protoplast thereof comprising increasing the expression of an RKD transcription factor in the wheat plant, plant part, cell or protoplast thereof, wherein the method comprises the steps above.

Suitably the method may be a method of increasing number of panicle branches and/or number of seeds per panicle in a rice plant, plant part, cell or protoplast thereof relative to a reference rice plant, plant part, cell or protoplast thereof comprising increasing the expression of an RKD transcription factor in the rice plant, plant part, cell or protoplast thereof, wherein the method comprises the steps above.

### Increase in Yield-Related Trait

The present invention relates to increasing a yield-related trait in a plant by increasing the expression of an RKD transcription factor. By ‘increasing a yield related trait’ it is meant that one or more yield related traits are increased relative to a reference plant, wherein a reference plant may otherwise be known as a control plant and is defined elsewhere herein.

The value of a yield-related trait of the reference plant, plant part, cell or protoplast thereof may appropriately be calculated as a mean value of several reference plants. The value of a yield-related trait of the modified plant, plant part, cell or protoplast thereof may appropriately be calculated as a mean value of several modified plants.

Suitably, where a yield-related trait is increased in the modified plant, plant part, cell or protoplast thereof relative to a reference plant, plant part, cell or protoplast thereof, the trait may optionally be increased by 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000% relative to the same yield-related trait in a reference plant, plant part, cell or protoplast.

Suitably, in all embodiments of the invention where a yield-related trait is increased in the modified plant, plant part, cell or protoplast thereof relative to a reference plant, plant part, cell or protoplast thereof, the trait may optionally be increased by 100% to 149%, 150% to 199%, 200% to 249%, 250%, to 299%, 300% to 349%, 350% to 399%, 400% to 449%, 450% to 499%, 500% to 599%, 600% to 699%, 700% to 799%, 800% to 899% or 900% to 1000% relative to the same yield-related trait in a reference plant, plant part, cell or protoplast.

Suitably, in some embodiments the modified plant, plant part, cell or protoplast thereof comprises a decrease in a different trait relative to a reference plant, plant part, cell or protoplast. Suitably, in some embodiments, the modified plant, plant part, cell or protoplast thereof has an increase in a yield-related trait and a decrease in a different trait relative to a reference plant, plant part, cell or protoplast. A different trait may refer to a trait not related to plant yield or any trait that is undesirable, for example a trait which negatively impacts plant yield.

Suitably, where a different trait is decreased in the modified plant, plant part, cell or protoplast thereof relative to a reference plant, plant part, cell or protoplast thereof, the trait may optionally be decreased by 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% relative to that of the same trait in a reference plant, plant part, cell or protoplast.

Suitably, in all embodiments of the invention where a different trait is decreased in the modified plant, plant part, cell or protoplast thereof relative to a reference plant, plant part, cell or protoplast thereof, the different trait may optionally be decreased by 99% to 90%, 89% to 80%, 79% to 70%, 69%, to 60%, 59% to 50%, 49% to 40%, 39% to 30%, 29% to 20%, 19% to 10%, 9% to 1% relative to that of the same trait in a reference plant, plant part, cell or protoplast. As above, the different trait of the reference plant part, cell or protoplast thereof may appropriately be calculated as a mean value of several reference plants. The different trait of the modified plant part, cell or protoplast thereof may appropriately be calculated as a mean value of several modified plants.

### Increasing the Expression of a RKD Transcription Factor

Suitably, increasing the expression of an RKD transcription factor may involve providing a plant, plant part, cell or protoplast thereof with an RKD transcription factor protein or a variant or active fragment thereof. Suitably, increasing the expression of an RKD transcription factor may involve providing a plant, part, cell or protoplast thereof with a polynucleotide encoding the RKD transcription factor, variant or active fragment thereof.

Suitably, the polynucleotide encoding the RKD transcription factor, variant or active fragment thereof may be a polynucleotide encoding a RKD transcription factor, variant or active fragment thereof and a regulatory sequence controlling expression thereof. Suitably the polynucleotide encoding the RKD transcription factor, variant or active fragment thereof may be an expression cassette comprising a polynucleotide encoding a RKD transcription factor, variant or active fragment thereof and a regulatory sequence controlling expression thereof. Suitably, the polynucleotide encoding the RKD transcription factor, variant or active fragment thereof may be DNA. Suitably, the polynucleotide encoding the RKD transcription factor, variant or active fragment thereof may be RNA. Suitably the polynucleotide may comprise more than one polynucleotide encoding a RKD transcription factor, variant or active fragment thereof and optionally one or more regulatory sequences.

Suitably providing the plant, plant part, cell or protoplast with an RKD transcription factor protein or a variant or active fragment thereof, or a polynucleotide encoding said RKD transcription factor, variant or active fragment thereof comprises introducing an RKD transcription factor protein or a variant or active fragment thereof, or a polynucleotide encoding said RKD transcription factor, variant or active fragment thereof into a plant, plant part, cell or protoplast.

Suitably providing or introducing the RKD transcription factor protein or a variant or active fragment thereof, or a polynucleotide encoding said RKD transcription factor, variant or active fragment thereof into the plant, plant part, plant cell or protoplast modifies the plant, plant part, cell or protoplast. Suitably genetically modifies the plant, plant part, cell or protoplast.

Suitably introducing may comprise transducing or transforming the plant, plant part, cell or protoplast with the RKD transcription factor protein or a variant or active fragment thereof, or a polynucleotide encoding said RKD transcription factor, variant or active fragment thereof.

Suitably therefore step (a) may comprise transforming or transducing a plant, plant part, cell or protoplast thereof with an RKD transcription factor or a variant or active fragment thereof, or a polynucleotide encoding said RKD transcription factor, variant or active fragment thereof.

Suitably step (a) may comprise increasing the expression of an RKD transcription factor or a variant or active fragment thereof in a plant, plant part, cell or protoplast thereof. Suitably step (a) may comprise overexpressing an RKD transcription factor or a variant or active fragment thereof in a plant, plant part, cell or protoplast thereof. Suitably which may be achieved by transforming or transducing a plant, plant part, cell or protoplast thereof with an RKD transcription factor or a variant or active fragment thereof, or a polynucleotide encoding said RKD transcription factor, variant or active fragment thereof.

Suitably step (a) may comprise modifying a plant, plant part, cell or protoplast thereof to increase the expression of an RKD transcription factor or a variant or active fragment thereof. Suitably step (a) may comprise modifying a plant, plant part, cell or protoplast thereof to overexpress an RKD transcription factor or a variant or active fragment thereof. Suitably which may be achieved by transforming or transducing a plant, plant part, cell or protoplast thereof with an RKD transcription factor or a variant or active fragment thereof, or a polynucleotide encoding said RKD transcription factor, variant or active fragment thereof.

In one embodiment, increasing the expression of a RKD transcription factor may involve providing a gene editing system for modifying an endogenous nucleic acid sequence encoding an RKD transcription factor or a regulatory sequence controlling expression thereof. Suitably providing the gene editing system to the plant, plant part, cell or protoplast comprises introducing the gene editing system into the plant, plant part, cell or protoplast. Suitably introducing the gene editing system into the plant, plant part, cell or protoplast may comprise transducing or transforming a plant, plant part, cell or protoplast with the gene editing system. Suitably in such an embodiment the plant, plant part, cell or protoplast is non-transgenic.

Suitable means of transducing or transforming a plant, plant part, cell or protoplast are discussed elsewhere herein.

Suitably step (a) may comprise increasing the expression of, or overexpression of, an RKD transcription factor or a variant or active fragment thereof in a plant, plant part, cell or protoplast thereof. Suitably which may be achieved by transforming or transducing a plant, plant part, cell or protoplast thereof with the gene editing system. Suitably the gene editing system is operable to increase expression of, or overexpress, an RKD transcription factor or a variant or active fragment thereof, suitably an endogenous RKD transcription factor or a variant or active fragment thereof.

Suitably step (a) may comprise modifying a plant, plant part, cell or protoplast thereof to increase the expression of, or overexpress, an RKD transcription factor or a variant or active fragment thereof. Suitably which may be achieved by transforming or transducing a plant, plant part, cell or protoplast thereof with the gene editing system. Suitably the gene editing system is operable to increase expression of, or overexpress, an RKD transcription factor or a variant or active fragment thereof, suitably an endogenous RKD transcription factor or a variant or active fragment thereof.

Suitably the method may further comprise a step of expressing the RKD transcription factor in the plant, plant part, cell or protoplast thereof. Suitably the method may further comprise a step of overexpressing the RKD transcription factor in the plant, plant part, cell or protoplast thereof. Suitably this step occurs after step (a) of providing, introducing, transforming or transducing the plant, plant part, cell or protoplast thereof. Suitably expressing, or overexpressing, the RKD transcription factor may occur automatically after step (a) or may comprise inducing expression of the RKD transcription factor. Suitable means of inducing expression are described elsewhere herein, but may include contacting the plant, plant part, cell or protoplast thereof with an inducer.

Suitably, where the RKD transcription factor expression is increased relative to reference plants, the expression of a RKD transcription factor in the modified plant, plant part, cell or protoplast thereof may optionally be increased by 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000% relative to the expression of RKD transcription factor in the reference plant, plant part, cell or protoplast thereof.

Suitably, where a RKD transcription factor expression is increased relative to reference plants, the expression of the RKD transcription factor in the modified plant, plant part, cell or protoplast thereof may optionally be increased by 101% to 149%, 150% to 199%, 200% to 249%, 250%, to 299%, 300% to 349%, 350% to 399%, 400% to 449%, 450% to 499%, 500% to 599%, 600% to 699%, 700% to 799%, 800% to 899% or 900% to 1000% relative to the expression of RKD transcription factor in the reference plant, plant part, cell or protoplast thereof (i.e. the control value).

Suitably, increasing the expression of a RKD transcription factor may refer to increasing, elevating or upregulating the level of RKD transcription factor expression in a plant, plant part, cell or protoplast such that it exceeds the level of RKD transcription factor expression in a reference plant, plant part, cell or protoplast.

In one embodiment, increasing, elevating or upregulating the level of RKD transcription factor expression in a plant, plant part, cell or protoplast as used herein may be regarded as overexpression of the RKD transcription factor. Suitably ‘overexpressing’ may be used interchangeably with any of these terms. Suitably when the RKD transcription factor is overexpressed, the level of expression thereof exceeds the level of RKD transcription factor expression in a reference plant, plant part, cell or protoplast. Suitably the level of expression may exceed the level of RKD transcription factor expression in a reference plant, plant part, cell or protoplast by any of the values mentioned above.

In one embodiment, increasing the expression of a RKD transcription factor may refer to ectopically expressing the RKD transcription factor in a plant, plant part, cell or protoplast. Suitably therefore, the expression of the RKD transcription factor may be increased due to the RKD transcription factor being ectopically expressed. By ‘ectopic’ expression it is meant that the RKD transcription factor is expressed in tissues or cells of a plant or a plant part, or in cells or protoplasts of a plant, in which it is not typically expressed in a reference plant. Suitably therefore ectopic expression is a form of increased expression. Suitably the RKD transcription factor may be ectopically expressed in vegetative tissues of a plant or plant part, or in vegetative cells or protoplasts of a plant. In some embodiments, the RKD transcription factor is a reproductive RKD transcription factor, as explained elsewhere herein, which is ectopically expressed in a pant, plant part, cell or protoplast. In some embodiments, the RKD transcription factor is an reproductive RKD transcription factor, as explained elsewhere herein, which is ectopically expressed in a vegetative tissue of a plant or plant part, or in vegetative cells or protoplasts of a plant.

Suitably step (a) of the method may comprise providing a vegetative tissue of a plant or plant part, or a vegetative cell or protoplast thereof with a reproductive RKD transcription factor or a variant or active fragment thereof, or a polynucleotide encoding said reproductive RKD transcription factor, variant or active fragment thereof.

Suitably step (b) of the method may comprise providing a vegetative tissue of a plant or plant part, or a vegetative cell or protoplast thereof with a gene editing system for modifying an endogenous nucleic acid sequence encoding a reproductive RKD transcription factor or a regulatory sequence controlling expression thereof.

Suitably therefore the method of the invention may comprise a method of increasing a yield-related trait in a plant, plant part, cell or protoplast thereof relative to a reference plant, plant part, cell or protoplast thereof comprising ectopically expressing an RKD transcription factor in the plant, plant part, cell or protoplast thereof. Suitably ectopically expressing a reproductive RKD factor in the plant, plant part, cell or protoplast thereof. Suitably ectopically expressing a reproductive RKD factor in vegetative tissues of a plant, plant part, cell or protoplast thereof. Suitably therefore the method of the invention may comprise a method of increasing a yield-related trait in a plant, plant part, cell or protoplast thereof relative to a reference plant, plant part, cell or protoplast thereof comprising ectopically overexpressing an RKD transcription factor in the plant, plant part, cell or protoplast thereof. Suitably ectopically overexpressing a reproductive RKD factor in the plant, plant part, cell or protoplast thereof. Suitably ectopically overexpressing a reproductive RKD factor in vegetative tissues of a plant, plant part, cell or protoplast thereof.

Suitably therefore the plant, plant part, cell or protoplast having increased RKD transcription factor expression is a modified, transgenic, or a transformed plant part, cell or protoplast. Suitably therefore the plant, plant part, cell or protoplast having ectopic RKD transcription factor expression is a modified, transgenic, or a transformed plant part, cell or protoplast.

Suitably a reference plant, plant part, cell or protoplast as referred to herein is a non-modified, non-transgenic, untransformed plant, plant part, cell or protoplast of the same species as the modified plant, plant part, cell or protoplast of the invention. The reference plant, plant part, cell or protoplast may be genetically equivalent to the modified plant, plant part, cell or protoplast, but unmodified. The reference plant, plant part, cell or protoplast may be a wild type plant, plant part, cell or protoplast of the same species as the modified plant, plant part, cell or protoplast. The level of a RKD transcription factor expression in a reference plant, plant part, cell or protoplast may be considered to be a control or reference value. Such a control or reference value may appropriately be calculated as a mean value of several reference plants, plant parts, cells or protoplasts. Equally, the level of RKD transcription factor expression in modified plants, plant parts, cells or protoplasts of the invention may be calculated as a mean value of several modified plants, plant parts, cells or protoplasts.

### Gene Editing System

The methods of the present invention allow for the modification of plants, plant parts, cells, or protoplasts to increase the expression of an RKD transcription factor by using a gene editing system, specifically by providing a gene editing system to the plants, plant parts, cells, or protoplasts which will target and modify an endogenous sequence encoding an RKD transcription factor or a regulatory sequence controlling expression thereof.

Suitably this gene editing embodiment is an alternative to providing the plant, plant part, cell or protoplast with an exogenous RKD transcription factor, or a variant or active fragment thereof, or a polynucleotide encoding said RKD transcription factor, variant or active fragment thereof.

A “gene editing system” as used in the context of the present invention may refer to any tool that is suitable for modifying genetic code of an organism. Gene editing techniques are known in the art. Suitable gene editing systems may be selected from the list comprising of CRISPR-CAS9, CRISPR-CAS12a, RNA interference, short-hairpin RNAs, TALENs, zinc-finger nucleases.

The terms “CRISPR-Cas” AND “gRNA” are well known to the person skilled in the art. CRISPR/Cas technology is described extensively in the literature (e.g. Cong et al. ‘Multiplex Genome Engineering Using CRISPR/Cas Systems’, Science, 15-2-2013: Vol. 339 no. 6121 pp. 819-823) and, inter alia, in the following patent documents: U.S. Pat. No. 8,697,359, US2010076057, WO2013/176772, U.S. Pat. No. 8,771,945, US2010076057, US2014186843, US2014179770, US2014179006, WO2014093712, WO2014093701, WO2014093635, WO2014093694, WO2014093655, WO2014093709, WO2013/188638, WO2013/142578, WO2013/141680, WO2013/188522, U.S. Pat. No. 8,546,553, WO2014/089290, and WO2014/093479. CRISPR/Cas systems can be obtained commercially from Sigma-Aldrich (St. Louis, MO, US) under the CRISPR/Cas Nuclease RNA-guided Genome Editing suite of products and services, or from Thermo Fisher Scientific, Inc. (Waltham, MA, US) under the GeneArt® CRISPR branded products and services.

The term “Zinc fingers” or “zinc finger domains” and “Zinc finger nuclease” are well known to the person skilled in the art. Zinc fingers are small protein structural motifs which make finger-like protrusions that make tandem contacts with their target molecule. Zinc finger-\dna code is well known to the person skilled in the art. Zinc fingers may be fused to DNA cleavage module such as a nuclease to make a zinc finger nuclease. ZFNs can be engineered to target and induce a DSB at any sequence. ZFN technology is described extensively in the literature and, inter alia, in the following patent documents: U.S. Pat. Nos. 6,479,626, 6,534,261, 6,607,882, 6,746,838, 6,794, 136, 6,824,978, 6,866,997, 6,933,113, 6,979,539, 7,013,219, 7,030,215, 7,220,719, 7,241,573, 7,241,574, 7,585,849, 7,595,376, 6,903,185, 6,479,626, 8,106,255, 20030232410, and 20090203140. ZFNs can be obtained commercially from Sigma-Aldrich (St. Louis, MO, US) under the CompoZr® Zinc Finger Nuclease Technology branded products and services.

The term “TALE domain” and “TALEN” are well known to the person skilled in the art. TALE domains are proteins which recognize DNA sequences through a central repeat domain consisting of a variable number of ˜34 amino acid repeats. Two critical amino acids (called RVDs) in each repeat bind specific DNA bases. The TALE-DNA code is well known to the one skilled in the art. One or more TALE domains may be fused to a DNA cleavage module such as a nuclease to make a TALEN. TALEN technology is described extensively in the literature and, inter alia, in the following patent documents: U.S. Pat. Nos. 8,420,782, 8,470,973, 8,440,431, 8,440,432, 8,450,471, 8,586,363, 8,697,853, EP2510096, U.S. Pat. Nos. 8,586,526, 8,623,618, EP2464750, US2011041195, US2011247089, US2013198878, WO2012/116274, WO2014110552, WO2014070887, WO2014022120, WO2013192316, and WO2010008562. TALENs can be obtained commercially from Thermo Fisher Scientific, Inc. (Waltham, MA, US) under the GeneArt® TALs branded products and services (formerly marketed under the Life Technologies brand).

In one embodiment the gene editing system is a CRISPR-Cas system, suitably a CRISPR-Cas9 system, a CRISPR-Cas12 system, a CRISPR-Cas13a system, or a CRISPR-Cas13b system. In one embodiment, the gene editing system is a CRISPR-Cas9 system.

Suitably in such an embodiment, the gene editing system comprises a Cas enzyme, optionally one or more further Cas proteins, and a DNA-targeting RNA. Suitably providing the gene editing system to a plant, plant part, cell or protoplast comprises providing a Cas enzyme or nucleic acid sequence encoding a Cas enzyme, and a DNA targeting RNA or a nucleic acid sequence encoding a DNA targeting RNA to the plant, plant part, cell or protoplast. As described elsewhere herein, providing may comprise introducing said components into the plant, plant part, cell or protoplast. Suitable means of introducing proteins and nucleic acids into cells are described elsewhere herein.

Suitably the DNA targeting RNA may be a guide RNA. Suitably the guide RNA may be a single molecule guide RNA or a dual molecule guide RNA. Suitably the DNA-targeting RNA targets a target region of an endogenous nucleic acid sequence. Suitably the target region in this case encodes an RKD transcription factor or a regulatory sequence controlling expression thereof. Suitably by targets it is meant that the DNA-targeting RNA comprises a sequence which is complementary to a target region of the endogenous nucleic acid sequence encoding an RKD transcription factor or a regulatory sequence controlling expression thereof, such that the DNA-targeting RNA is capable of directing the Cas enzyme to bind to the target region. Suitably the DNA targeting RNA may also comprise a Cas-binding region in order to bind to the Cas protein(s) and target it to the target region of an endogenous nucleic acid sequence. Suitably the target region is adjacent to a Protospacer Adjacent Motif (PAM), suitably the target region is immediately adjacent to a PAM, suitably the target region is immediately downstream of a PAM. Suitable PAM sequences are known in the art but may be for example 5′-NGG-3′. Suitable tools in the art are known to design guide RNA sequences which will bind to a desired target region which is adjacent to a PAM.

Suitably, the target region is a regulatory sequence controlling expression of an endogenous RKD transcription factor. Suitably therefore the DNA-targeting RNA comprises a region which is complementary to a regulatory sequence controlling expression of an endogenous RKD transcription factor. Suitably the regulatory sequence controlling expression of an endogenous RKD transcription factor may be a promoter, repressor, enhancer etc. Suitably the regulatory sequence is operably linked to the polynucleotide sequence encoding the endogenous RKD transcription factor. Suitably, the Cas enzyme is capable of editing the regulatory sequence to modulate a pattern of expression of an endogenous RKD transcription factor in a modified plant, plant part, cell or protoplast of the present invention. Suitably, the pattern of expression of the RKD transcription factor in the modified plant, plant part, cell or protoplast may be modulated from embryonic tissue expression to vegetative tissue expression.

Editing of the regulatory sequence may be used in order to achieve an increase in RKD transcription factor expression in vegetative tissues in the modified plant, plant part, cell or protoplast of the present invention compared to a reference plant. Editing of the regulatory sequence may be used in order to achieve an increase in reproductive RKD transcription factor expression in vegetative tissues in the modified plant, plant part, cell or protoplast of the present invention compared to a reference plant. Suitably, the RKD transcription factor expression in the modified plant, plant part, cell or protoplast is increased in vegetative tissues compared to a reference plant. Suitably, the reproductive RKD transcription factor expression in the modified plant, plant part, cell or protoplast is increased in vegetative tissues compared to a reference plant.

Suitably the Cas enzyme may edit the target region by cleaving the target sequence. Suitably after cleavage of the target region, one or more modifications may be introduced into the target region by repair. Suitably by non-homologous end joining or via homology directed repair mediated by cellular machinery. Suitably modifications such as deletions, or insertions may be introduced into the target region. Suitably the modifications may have the desired effects described above.

### RKD Transcription Factor Mechanisms

Without wishing to be bound by theory, the inventors have made the surprising discovery that increasing the expression of RKD transcription factors may increase yield related traits in plants by positively regulating epigenetic regulators such as histone methyltransferases and activating cell reprogramming through chromatin remodelling.

Experimental evidence suggests that ectopic TaRKD1 expression induces cell reprogramming and activates cell cycle by increasing chromatin-related factors such as factors involved in increasing chromatin accessibility; chromatin assembly; DNA packaging; and protein-DNA complex assembly.

Suitably, the modified plant, plant part, cell or protoplast thereof having an increase in a yield-related trait as obtained by the methods described herein may also have increased expression of one or more chromatin-related factors. Suitably, the modified plant, plant part, cell or protoplast thereof having an increase in a yield-related trait as obtained by the methods described herein may also have increased expression of one or more factors involved in chromatin accessibility; chromatin assembly; DNA packaging; and/or protein-DNA complex assembly relative to a reference plant. Suitably therefore the method of the invention may be a method for increasing the expression of one or more factors involved in chromatin accessibility; chromatin assembly; DNA packaging; and/or protein-DNA complex assembly in a plant, plant part, cell or protoplast thereof relative to a reference plant. Suitably the method may comprise obtaining a plant, plant part, cell or protoplast thereof having an increased expression of one or more factors involved in chromatin accessibility; chromatin assembly; DNA packaging; and/or protein-DNA complex assembly compared to a reference plant.

The inventors have further found that increased expression of RKD transcription factors is associated with increased levels of Histone H4. Suitably, providing or increasing expression of an RKD transcription factor may result in upregulation of histone H4. Histone H4, a histone protein involved in the chromatin structure, is known to be associated with cell division and is highly expressed in dividing tissues such as meristems and embryos. The inventors have found that increased expression of RKD transcription factors induces cell reprogramming via controlling chromatin accessibilities and initiates meristems through activating cell cycles. Specifically, increased expression of RKD transcription factors in some embodiments of the present invention may result in increased histone modification factors, cell cycling factors and DNA methylation factors. Suitably, the modified plant, plant part, cell or protoplast thereof having an increase in a yield-related trait as obtained by the methods described herein may also have increased expression of one or more histone modification factors, cell cycling factors and/or DNA methylation factors relative to a reference plant. Suitably therefore the method of the invention may be a method for increasing the expression of one or more histone modification factors, cell cycling factors and/or DNA methylation factors in a plant, plant part, cell or protoplast thereof relative to a reference plant. Suitably the method may comprise obtaining a plant, plant part, cell or protoplast thereof having an increased expression of one or more histone modification factors, cell cycling factors and/or DNA methylation factors compared to a reference plant.

Suitably, the modified plant, plant part, cell or protoplast thereof having an increase in a yield-related trait as obtained by the methods described herein may have increased histone H4 and/or H3K27 methylation relative to a reference plant. Suitably therefore the method of the invention may be a method for increasing the histone H4 and/or H3K27 methylation in a plant, plant part, cell or protoplast thereof relative to a reference plant. Suitably the method may comprise obtaining a plant, plant part, cell or protoplast thereof having increased histone H4 and/or H3K27 methylation compared to a reference plant.

In some embodiments, the present invention may comprise use of increased histone H4 and/or H3K27 methylation as a selectable marker for selecting plants having an increase in a yield related trait. Suitably selecting those plants having increased histone H4 and/or H3K27 methylation will select for plants having an increase in expression of an RKD transcription factor. Suitably said plants may have a mutation within a polynucleotide encoding an RKD transcription factor.

Surprisingly, the inventors have further found that RKD transcription factor expression upregulates histone methyltransferases, and increases binding of RKD transcription factors to meristemic determinacy factor promoters which induces increased meristemic determinacy factor expression.

Accordingly, in some embodiments, the modified plant, plant part, cell or protoplast thereof having an increase in a yield-related trait as obtained by the methods described herein may further have increased expression of one or more meristemic determinacy factors. Suitably, meristemic determinacy factors may include genes involved in meristem maintenance. Suitably, genes involved in meristem maintenance include but are not limited to wuschel (WUS), GROWTH-REGULATING FACTOR 4 (GRF4) and its cofactor GRF-INTERACTING FACTOR 1 (GIF1), auxin response factor 3 (ARF3), apetala2 (AP2), auxin, leafy (LFY), YUC4, shoot meristemless (STM), IPT7, cytokinin (CK), clavata 3 (CLV3), far-red elongated hypocotyl 3 (FHY3), fruitfull (FUL), squamosa promoter binding protein-like (SPL), argonaute1 (AGO1), argonaute10 (AGO10), class III homeodomain-leucine zipper (HD-ZIP III), miRNA156, miRNA 164a/b/c, miRNA 165/166, miR172, shoot meristemless (STM), GA20 oxidase (G20ox), cup-shaped cotyledon 1/2/3, asymmetric leaves 1/2 (AS1/2), teosinte branched1/cycloidea/pcf1 (TCP), Primordium identity factors (PrlFs), KNU, powerdress (PWR), CRC, and ramosa 3.

Suitably therefore the method of the invention may be a method for increasing the expression of one or more meristemic determinacy factors in a plant, plant part, cell or protoplast thereof relative to a reference plant. Suitably the method may comprise obtaining a plant, plant part, cell or protoplast thereof having increased expression of one or more meristemic determinacy factors compared to a reference plant. In other embodiments, the modified plant, plant part, cell or protoplast thereof having an increase in a yield-related trait as obtained by the methods described herein may have upregulated expression of one or more histone methyltransferases. Suitably therefore the method of the invention may be a method for increasing the expression of one or more histone methyltransferases in a plant, plant part, cell or protoplast thereof relative to a reference plant. Suitably the method may comprise obtaining a plant, plant part, cell or protoplast thereof having increased expression of one or more histone methyltransferases compared to a reference plant.

The inventors have further found that overexpression of RKD transcription factors may also result in an increase in the expression of trehalose synthase. Suitably, upregulation of trehalose-6-phosphate (Tre-6-p) synthases decreases meristematic determinacy, causing increased meristem formation and maintenance.

Accordingly, in some embodiments, the modified plant, plant part, cell or protoplast thereof having an increase in a yield-related trait as obtained by the methods described herein may further have increased expression of trehalose synthase relative to a reference plant.

Suitably therefore the method of the invention may be a method for increasing the expression of trehalose synthase/trehalose synthesis in a plant, plant part, cell or protoplast thereof relative to a reference plant. Suitably the method may comprise obtaining a plant, plant part, cell or protoplast thereof having increased trehalose synthase expression/trehalose synthesis compared to a reference plant.

In some embodiments, the present invention may comprise use of increased trehalose synthesis as a selectable marker for selecting plants having an increase in a yield related trait. Suitably selecting those plants having increased trehalose synthesis will select for plants having an increase in expression of an RKD transcription factor. Suitably said plants may have a mutation within a polynucleotide encoding an RKD transcription factor.

### Plant

The plant may be any plant species. The plant may be a plant which is typically used as a crop, whether for human food, animal food, energy or other purposes. In some embodiments, the plant is a Monocotyledon (monocot). In some embodiments, the plant is a Dicotyledon (dicot) plant. Suitably, monocot plants include grasses or grass-like plants. Dicots include trees, shrubs, soybean, and flowering plants such as roses, foxglove and alcea.

In some embodiments, the plant is an agricultural plant species of economic significance. In some embodiments, the plant is a crop plant.

Suitable plants for use in the present invention include crops and plants of agricultural, horticultural, or economic significance. Suitable plants may include any of the following or parts, cells or protoplasts thereof: Musa textilis, Medicago sativa, Prunus dulcis, Pimpinella anisum, Malus sylvestris, Prunus armeniaca, Areca catechu, Arracacia xanthorhiza, Maranta arundinacea, Cynara scolymus, Helianthus tuberosus, Asparagus officinalis, Persea americona, Pennisetum americanum, Vigna subterranean, Musa paradisiaca, Hordeum vulgare, Phaseolus vulgaris, Phaseolus vigna spp., Beta vulgaris, Citrus bergamia, Rubus spp., Piper nigrum, Acacia mearnsii, Vaccinium spp., Bertholletia excelsa, Artocarpus altilis, Vicia faba, Brassica oleracea botrytis, Sorghum bicolor, Brassica oleracea gemmifera, Fagopyrum esculentum, Brassica oleracea capitate, Brassica rapa, Brassica spp., Theobroma cacao, Cucumis melo, Carum carvi, Elettaria cardamomum, Cynara cardunculus, Ceratonia siliqua, Daucus carota, Anacardium occidentale, Manihot esculenta, Ricinus communis, Brassica oleracea botrytis, Apium graveolens, Sechium edule, Prunus spp., Castanea sativa, Cicer arietinum, Cichorium intybus, Cichorium intybus, Capsicum spp., Cinnamomum verum, Cymbopogon nardus, Citrus medica, Citrus veticulata, Trifolium spp., Syzygium aromaticum, Cocos nucifera, Colocasia spp.; Xanthosoma spp., Coffee spp., Cola spp., Brassica napus, Zea mays, Valerianella locusta, Gossypium spp., Vigna unguiculate, Vaccinium spp., Lepidium sativum, Cucumis sativus, Ribes spp., Annona reticulata, Colocasia esculenta, Phoenix dactylifera, Moringa oleifera, Phaseolus spp., Allium sativum, Allium cepa, Pisum sativum, Triticum durum, Xanthosoma spp.; Colocasia spp., Solanum melongena, Cichorium endivia, Lygeum spartum, Foeniculum vulgare, Trigonella foenumgraecum, Ficus carica, Corylus avellane, Furcraea macrophylla, Linum usitatissimum, Phormium tenax, Pelargonium spp.; Geranium spp., Zingiber officinalis, Langenaria spp; Cucurbita spp., Cicer arietinum, Citrus paradise, Vitis vinifera, Lygeum spartum, Dactylis glomerata, Arachis hypogaea, Psidium guajava, Corylus avellane, Cannabis sativa, Crotalaria juncea, Agave fourcroydes, Lawsonia inermis, Humulus lupulus, Armoracia Rusticana, Indigofera tinctorial, Jasminum spp., Corchorus spp., Brassica oleracea acephala, Ceiba pentandra, Hibiscus cannabinus, Brassica oleracea gongylodes, Lavandula spp., Allium ampeloprasum, Citrus limon, Cymbopogon citratus, Lens culinaris, Lespendeza spp., Lactuca sativa, Glycyrrhiza glabra, Citrus aurantifolia, Citrus limetta, Linum usitatissimum, Litchi chinensis, Eriobotrya japonica, Lupinus spp., Macadamia spp., Myristica fragrans, Agave atrovirens, Citrus reticulata, Mangifera indica, Manihot esculenta, Secale cereal, Mespilus germanica, Cucumis melo, Penicum miliaceum, Eleusine coracana, Setaria italica, Echinochloa crusgalli, Eleusine coracana; Mentha spp., Morus spp., Morus alba, Agaricus spp.; Pleurotus spp. Volvariella, Brassica nigra; Sinapis alba, Prunus persica, Phormium tenax, Guizotia abyssinica, Myristica fragrans, Avena spp., Elaeis guineensis, Abelmoschus esculentus, Olea europea, Papaver somniferum, Citrus sinensis, Citrus aurantium, Dactylis glomerate, Metroxylon spp., Borassus flabellifer, Carica papaya, Pastinaca sativa, Pyrus communis, Pisum sativum, Carya illinoensis, Capsicum annuum, Diospyros kaki; Diospyros virginiana, Cajanus cajan, Ananas comosus, Pistacia spp., Prunus domestica, Punica granatum, Citrus grandis, Solamum tuberosum, Ipomoea batatas, Cucurbita spp., Chrysanthemum cineraraiefolium, Aspidosperma spp., Cydonia oblonga, Cinchona spp., Chenopodium quinoa, Raphanus sativus (including Cochlearia armoracia), Boehmeria nivea, Agrostis spp., Boehmeria nivea, Rheum spp., Oryza sativa; Oryza glaberrima, Rose spp., Hevea brasiliensis, Secale cereal, Lolium spp., Carthamus tinctorius, Metroxylon spp., Onobrychis viciifolia, Valerianella locusta, Tragopogon porrifolius, Achras sapota, Citrus reticulata, Brassica ileracea capitate, Scorzonera hispanica, Sesamum indicum, Butyrospermum paradoxum, Agave sislana, Citrus aurantifolia, Glycine max, Triticum spelta, Spinacia oleracea, Secale cereal, Cucurbita spp., Fragaria spp., Sorghum bicolor Sudanense, Saccharum officinarum, Helianthus annuus, Crotalaria juncea, Citrus limetta, Iopmoea batatas, Citrus reticulata, Xanthosoma sagittifolium, Manihot esculenta, Colocasia esculenta, Camellia sinensis, Eragrostis abyssinica, Phleum pratense, Nicotiana tabacum, Lycopersicum esculentum, Lotus spp., Aleurites spp., Brassica rapa, Urena lobate, Vanilla planifolia, Vicia sativa, Juglans spp., Citrullus lanatus, Acacia mearnsii, Triticum spp., Hordeum spp., Dioscorea spp., and Ilex paraguariensis.

Suitable plants include the Poaceae family, suitably of the BOP clade and/or PACMAD clade. In a preferred embodiment, the plant genus is wheat (Triticinae), rice (Oryza), barley (Hordeum vulgare), oat (Avena sativa), maize (Zea mays) or sorghum (Sorghum).

Wheat may be any variety of wheat (Triticinae). Suitably, varieties of wheat include but are not limited to durum, spelt, emmer, hard red winter, hard red spring, soft red winter, soft white and hard white. In one embodiment, the plant is Triticum aestivum.

Rice may be any variety of rice (Oryza). Suitably, varieties of rice include but are not limited to cultivated varieties such as Oryza sativa (Asian rice), Oryza glaberrima (African rice) as well as wild varieties. In one embodiment, the plant is a Oryza sativa.

Barley may be any variety of barley (Hordeum). Suitably, varieties of barley include but are not limited to cultivated varieties such as two-row or common barley (Hordeum distichon) and six-row barley (Hordeum vulgare) as well as wild varieties such as Hordeum spontaneum. In one embodiment, the plant is Hordeum distichon. In another embodiment, the plant is Hordeum vulgare.

Oat may be any variety of oat (Avena). Suitably, varieties of oat include but are not limited to cultivated varieties such as common oats (Avena sativa), hull-less oats (Avena nuda) and black oats (Avena strigosa), as well as wild varieties. In one embodiment, the plant is Avena sativa.

Maize may be any variety of maize (Zea). Suitably, varieties of maize include but are not limited to cultivated varieties such as flour corn (Zea mays amylacea), sweetcorn (Zea mays saccharate; and Zea mays rugosa), striped maize (Zea mays japonica), popcorn (Zea mays everta) as well as wild varieties. In one embodiment the plant is Zea mays amylacea.

Sorghum may be any variety of sorghum (Sorghum). Suitably, varieties of sorghum include but are not limited to cultivated varieties such as cultivated sorghum (Sorghum bicolor), Sorghum burmahicum, Sorghum amplum and Sorghum trichocladum. In one embodiment, the plant is Sorghum bicolor.

### Plant Part or Material

The present invention may include any plant biomass derived from a plant as described herein. Any reference to a ‘plant’ herein should be taken as encompassing a part of a plant, including one or more cells of a plant.

The term “plant part” may refer to any component of a plant. Examples of plant parts include but are not limited to leaves, stems, leaf sheathes, flowers, roots, tubers, seeds, rhizomes, grain, embryo, pollen, ovules, siliques, pods, ears, cobs, husks, stalks, fruits, root tips, anthers, pericarp, glumes, peduncles, silk, tissue or cells.

A plant part may also refer to any materials derived from a plant which may comprise plant products including flour, fruit, meal, timber and/or wood.

Suitably, according to one aspect of the invention there is provided a seed, or other plant part or plant material derived from the modified plant of the first and/or second aspects.

Other plant parts include but are not limited to leaves, stems, leaf sheathes, flowers, roots, tubers, seeds, rhizomes, grain, embryo, pollen, ovules, siliques, pods, ears, cobs, husks, stalks, fruits, root tips, anthers, pericarp, glumes, peduncles, silk, tissue or cells, for example.

Plant materials are derived from a plant, and include but are not limited to plant products such as flour, fruit, meal, timber and/or wood for example.

### Recombinant Polynucleotide

Suitably, the polynucleotide encoding said RKD transcription factor may be a recombinant polynucleotide.

Suitably, the recombinant polynucleotide encoding the RKD transcription factor encodes one or more of a plurality of RKD transcription factors as described herein. In one embodiment, the recombinant polynucleotide encoding the or each RKD transcription factor may comprise or consist of one or more sequences encoding an RKD transcription factor as described hereinabove. In one embodiment, the recombinant polynucleotide encoding the or each RKD transcription factor may comprise or consist of one or more sequences according to any one of SEQ ID NO: 1, 4, 6-37 or a functional variant thereof. In a preferred embodiment, the recombinant polynucleotide encodes one RKD transcription factor. In a preferred embodiment, the recombinant polynucleotide encoding the RKD transcription factor may comprise or consist of a sequence according to any one of SEQ ID NO: 1, 4, 6-37 or a functional variant thereof.

Polynucleotides which encode an RKD transcription factor for use in invention may be wholly or partially synthetic and may include, but are not limited to, DNA, cDNA and RNA including but not limited to mRNA, rRNA, tRNA, ncRNA, lncRNA and miRNA.

Polynucleotides encoding an RKD transcription factor for use in the invention can be readily prepared by the skilled person using techniques which are well known to those skilled in the art, such as those described in Sambrook et al. “Molecular Cloning”, A laboratory manual, Cold Spring Harbor Laboratory Press, Volumes 1-3, 2001 (ISBN-0879695773), and Ausubel et al. Short Protocols in Molecular Biology. John Wiley and Sons, 4th Edition, 1999 (ISBN-0471250929). Said techniques include (i) the use of the polymerase chain reaction (PCR) to amplify samples of nucleic acid, (ii) chemical synthesis, or (iii) preparation of cDNA sequences.

Polynucleotides encoding RKD transcription factors may be generated and used in any suitable way known to those skilled in the art, including taking polynucleotides, identifying suitable restriction enzyme recognition sites either side of the portion to be expressed, and cutting out said portion from the polynucleotide. The excised portion may then be operably linked to a suitable promoter and expressed in a suitable expression system, such as a commercially available expression system. Alternatively, the relevant portions of polynucleotides can be amplified by using suitable PCR primers. Modifications to the polynucleotide sequences can be made by using site directed mutagenesis.

In some embodiments, the recombinant polynucleotide may additionally comprise a promoter region. The promoter may be a naturally occurring or a synthetic promoter. Suitable promoters may be plant-, bacteria-, fungal- or virus derived or from any other suitable organism providing that they produce the desired expression pattern in the recipient plant. In a preferred embodiment, the promoter is a plant promoter. Suitable promoters need not be monocot or dicot derived, and could in principle be derived from any plant species. Preferably, promoter sequences may be derived from the same species of plant as the plant being modified, suitably therefore the promoter is a homogenous promoter. In one embodiment the plant to be modified is wheat or rice, therefore the promoter will be derived from wheat or rice.

It will be understood that a range of promoter sequences could in principle be used to drive the expression of the RKD transcription factor polynucleotide sequences disclosed herein within a plant, depending on the desired expression pattern. Commonly, strong promoters capable of producing a high-level of mRNA transcript of the RKD transcription factor in the host plant will be used. In particular, those capable of driving high-levels of mRNA transcript of the RKD transcription factor in the specific tissue of interest, will be selected for use in accordance with the present invention. Suitable promoter sequences may include but are not limited to mannopine synthase, nopaline synthase, and octopine synthase; alcohol dehydrogenase promoter from Zea mays; light inducible promoters such as ribulose-biphosphate-carboxylase small subunit gene from various species and the major chlorophyll a/b binding protein gene promoter; histone promoters, actin promoters; Zea mays ubiquitin 1 promoter; 35S and 19S promoters of cauliflower mosaic virus; developmentally regulated promoters such as the waxy, zein, or bronze promoters from Zea mays; as well as synthetic or other natural promoters including those promoters exhibiting organ-specific expression or expression at specific plant development stages, such as the alpha-tubulin promoter. Preferred promoters for driving polynucleotide expression in recipient plants will include those which generate constitutive expression, for example the maize ubiquitin promoter (ZmUbi).

Suitably, in some embodiments, the promoter may a viral promoter. Suitably, the viral promoter may be selected from Cauliflower mosaic virus (CaMV) 35S promoter, Cassava vein mosaic virus (CsVMV) promoter, Sugarcane bacilliform badnavirus (ScBV) promoter, figwort mosaic virus (FMV) promoter, and Cotton leaf curl Burewala virus (CLCuBuV) promoter. In some embodiments, the promoter may be a plant promoter. Suitably, the plant promoter may be selected from plant ubiquitin (Ubi) promoter pine promoter, Zea mays ubiquitin (ZmUbi) promoter, rice actin 1 (Act-1) promoter and maize alcohol dehydrogenase 1 (Adh-1) promoter. In a preferred embodiment, the plant promoter is ZmUbi.

In some embodiments, the promoter is a plant promoter that drives expression in meristematic tissues.

A constitutive promoter is active in a cell in all circumstances. Preferably a constitutive promoter is selected from Cauliflower mosaic virus (CaMV) 35S, pine promoters, plant ubiquitin (Ubi), rice actin 1 (Act-1) and maize alcohol dehydrogenase 1 (Adh-1).

In some embodiments, CaMV 35S is a preferred constitutive promoter in dicots. In some embodiments, Maize Ubi (ZmUbi) and rice Act-1 are preferred promoters in monocots.

The skilled person will also appreciate that constitutive expression may not always be desired. Suitably, the promoter may be inducible.

Inducible promoters allow production of the expression product to be induced at a desired point in time, which is useful in many ways. An inducible promoter allows the cells to be grown to a specific density or number before inducing production of the expression product and harvesting. Inducible promoters can also be used to express co-factors which enhance the yield, potency or the stability of the expression product. Suitably, inducible promoters may direct expression of the RKD transcription factor in response to environmental, chemical or developmental cues, such as temperature, light, chemicals, drought, heat stress, and other stimuli such as alcohol, tetracycline, dexamethasone, estradiol and pathogenesis related protein induction.

Suitably, inducible promoters may be selected from light responsive promoters, stress responsive promoters, plant hormonal responsive promoters, sucrose responsive promoters, low-oxygen responsive promoters, chemically inducible promoters. For example, chemically inducible promoters may include an ethanol-inducible promoter, a dexamethasone-inducibile promoter or an estradiol-inducible promoter. Suitably the inducible promoter may be plant growth hormone inducible, for example IAA inducible, thereby inducing expression of the RKD transcription factor when the plant is growing. Suitably inducing RKD transcription factor during active growth of the plant increases yield related traits as the relevant yield related parts of the plant develop.

### Vector

Polynucleotide sequences encoding an RKD transcription factor for use in the invention may be provided as an expression cassette which may comprise at least one polynucleotide as described above for expression of the RKD transcription factor polynucleotide in a plant, plant part, cell or protoplast to increase the yield-related trait and one or more additional sequences provided as part of the expression cassette.

Suitable expression cassettes for use in the present invention may be constructed by standard techniques known in the art, to comprise 5′ and 3′ regulatory sequences.

Suitably the additional sequences may comprise regulatory sequences. Such regulatory sequences may be capable of influencing transcription or translation of a gene or gene product, for example in terms of initiation, accuracy, rate, stability, downstream processing and mobility. Examples of regulatory sequences include promoters (discussed above), 5′ and 3′ UTR's, enhancers (e.g., VP16 transactivation domain), transcription factor or protein binding sequences, start sites and termination sequences, ribosome binding sites, recombination sites, polyadenylation sequences, sense or antisense sequences. They may be DNA, RNA or protein. The regulatory sequences may be operably linked to the sequences of interest, i.e. to the polynucleotide sequence(s) encoding the RKD transcription factor. The regulatory sequences may be plant-, bacteria-, fungi- or virus-derived, and preferably may be derived from the same species of plant as the plant being modified i.e. homologous. Such elements may be included in the expression construct to obtain the optimal expression and function of RKD transcription factors in the plant or plant material.

Suitably, the expression cassette may also contains one or more restriction sites, to enable insertion of the nucleotide sequence and/or a regulatory sequence into the plant genome, at pre-selected loci. Also provided on the expression cassette may be transcription and translation initiation regions, to enable expression of the incoming genes, transcription and translational termination regions, and regulatory sequences. These sequences may be native to the plant being transformed, or may be heterologous. The expression cassettes may be a bi- or multi-functional expression cassette which functions in multiple hosts. Furthermore, as the application requires, the expression vector may further comprise additional elements which influence expression of the protein of interest in the host plant. Such regulatory sequences and elements can permit a skilled person to select in a predetermined manner a desired expression pattern in a host plant of interest depending on the application concerned.

Suitably, in one aspect of the present invention there is provided a recombinant polynucleotide or an expression cassette as set out above comprised on a vector, suitably an expression vector e.g., an expression vector adapted for expression in a plant cell.

Suitable vectors may be selected from: agrobacterium vectors, tobacco mosaic virus (TMV) vector, potato virus X vector, and cowpea mosaic virus vector.

Suitable expression vectors may comprise additional sequences encoding one or more selectable markers. Suitably a selectable marker allows for the selection of successful expression of said vector in a plant cell and/or under particular conditions. Suitable selectable markers may enable visualisation of plants expressing the RKD transcription factor (e.g., fluorescent markers such as GFP, RFP, YFP, CFP, EGFP, EYFP, DsRed, RFP1 and mCherry). Suitable selectable markers may also comprise herbicide resistance genes which may confer resistance to herbicides such as sulfonylureas, imidazolines, triazolopyrimidines, and pyrimidyloxybenzoatesin in plants expressing the RKD transcription factor.

The expression vector may also comprise one or more regulatory sequences as described above. The regulatory sequences may be plant-, bacteria-, fungal- or virus derived, and preferably may be derived from the same species of plant as the plant being modified to achieve optimal function of the polynucleotide encoding RKD transcription factors in the recipient plant background and to reduce unwanted effects.

### Method of Producing a Modified Plant

In one aspect of the invention, there is provided a method of producing a modified plant, wherein the method comprises:
Step (a) providing an RKD transcription factor or a variant or active fragment thereof, or a polynucleotide which encodes an RKD transcription factor, variant or active fragment thereof to a plant, plant part, cell or protoplast.
Suitably, the polynucleotide may be comprised in an expression cassette as above or a vector as described above. Alternatively, step (a) comprises providing a gene editing system to a plant, plant part, cell or protoplast which is suitable to edit an endogenous sequence encoding an RKD transcription factor. Suitably the gene editing system may be encoded by one or more polynucleotides which may be comprised in an expression cassette or vector as described above.

‘Providing’ encompasses introducing into the plant, plant part, cell or protoplast thereof a RKD transcription factor protein, a polynucleotide encoding a RKD transcription factor which may be comprised in an expression cassette or vector, or the components of a gene editing system which may be comprised in an expression cassette or vector. In some embodiments, the RKD transcription factor protein, polynucleotide encoding a RKD transcription factor, expression cassette, or gene editing system may be introduced by transformation or transduction.

Plants transformed with a polynucleotide or expression cassette, or vector of the invention, may be produced by standard techniques for the genetic manipulation of plants which are known in the art. DNA may be introduced into plant cells using any suitable technology, such as gene transfer via a disarmed Ti-plasmid vector carried by Agrobacterium tumefaciens, using Agrobacterium sp.-mediated transformation, vacuum infiltration, floral dip, spraying, particle or microprojectile bombardment, protoplast transformation, electroporation, microinjection, electrophoresis, pollen-tube pathway, silicon carbide- or liposome-mediated transformation, uptake by the roots, direct injection into the xylem or phloem or other forms of direct DNA uptake. Microprojectile bombardment, electroporation and direct DNA uptake are preferred where Agrobacterium is inefficient or ineffective. Alternatively, a combination of different techniques may be employed to enhance the efficiency of the transformation process, e.g., bombardment with Agrobacterium-coated microparticles or microprojectile bombardment to induce wounding followed by co-cultivation with Agrobacterium.

In one embodiment polynucleotide encoding an RKD transcription factor, or components of a gene editing system, may be directly introduced into plant cells using microprojectile bombardment. Preferably, transformation using microprojectile bombardment will include the acceleration of gold particles, coated with plasmid DNA (e.g., pBRACT214 gateway vector, or other suitable vectors including pBract202, pBract203, pBract208, pBract209, pBract204, pBract207, pBract210, pBract211 or pBract001) comprising a polynucleotide encoding an RKD transcription factor, or components of a gene editing system, and one or more regulatory sequences.

In one embodiment, a modified plant, plant part, or cell may be produced in accordance with the methods provided herein, where the RKD transcription factor, polynucleotide encoding the RKD transcription factor, gene editing system, polynucleotide encoding the gene editing system, an expression cassette or the expression vector as described above are applied directly onto the surface of the plant, part or cell to produce a modified plant, part, cell thereof having an increase in a yield-related trait. The expression vector may also be comprised in a composition. A composition may comprise the expression vector and a suitable carrier. Typically, the RKD transcription factor, polynucleotide encoding RKD transcription factors, gene editing system, polynucleotide encoding the gene editing system, expression cassette or expression vector or composition may be directly applied to the plant, part or cell, for example by rub-inoculation or by spraying directly on to the plant material, for example onto leaves, stems or roots of the plant during vegetative phase although other equally feasible methods of application will be known in the art. It is envisaged that the RKD transcription factor, polynucleotide encoding RKD transcription factor, gene editing system, polynucleotide encoding the gene editing system, expression cassette or the expression vector or composition may also be applied indirectly to the medium (e.g., soil or water) in which the plants, parts or cells are grown.

Transformation by direct application of plants, parts, or cells in accordance with the methods of the invention may involve a single application of the RKD transcription factor, polynucleotide encoding RKD transcription factor, gene editing system, polynucleotide encoding the gene editing system, expression cassette or the expression vector or composition either to the plant, part or cell or to the growth medium. However, it will be understood that treatment may alternatively involve multiple applications of the same, or indeed combinations thereof. Where multiple (i.e., two or more) different RKD transcription factors, polynucleotides encoding RKD transcription factors, gene editing systems, polynucleotides encoding the gene editing system, expression cassettes or expression vectors or compositions are applied to the same plant, these may be applied simultaneously, separately (in any order) or sequentially.

In some embodiments, the RKD transcription factor, polynucleotide encoding RKD transcription factor, gene editing system, polynucleotide encoding the gene editing system, expression cassette or the expression vector or composition disclosed herein are typically provided to the plant or plant part, or cell or protoplast in the form of an aqueous solution to produce a modified plant, part, cell or protoplast thereof having an increase in a yield-related trait. However, the RKD transcription factor, polynucleotide encoding RKD transcription factor, gene editing system, polynucleotide encoding the gene editing system, expression cassette or expression vector or composition disclosed herein may also be provided to the plant, plant part, cell or protoplast in solid form such as a powder, dust or in granular form and combinations thereof.

In a further aspect of the invention, there is provided the use of a composition comprising a polynucleotide encoding an RKD transcription factor, one or more polynucleotides encoding a gene editing system, or any of the expression vectors or cassettes comprising a polynucleotide encoding an RKD transcription factor or gene editing system as disclosed herein (and combinations thereof), for expressing an RKD transcription factor in a plant, plant part, cell or protoplast to increase a yield-related trait as compared to a reference plant, plant part, cell or protoplast. Suitably for ectopically expressing an RKD transcription factor in a plant, plant part, cell or protoplast to increase a yield-related trait. Suitably the composition is applied to the plant, plant part, cell or protoplast. Suitably the composition is applied to plant, part, cell or protoplast by rub-inoculation or by spraying or by uptake from the growth medium. Suitably the composition as disclosed herein is applied to the plant, part, cell or protoplast, or to the growth medium as an aqueous solution.

For convenience, the compositions disclosed herein may comprise other active ingredients used for the treatment of plants, for example the composition may comprise other agrochemical products such as fertilisers, herbicides, anti-bacterial or anti-fungal agents and/or pesticides.

Whole plants, plant material or plant parts may be stably or transiently transformed as desired, wherein stable transformation refers to polynucleotides which become incorporated into the plant host chromosomes such that the host genetic material may be permanently and heritably altered and the transformed cell may continue to express traits caused by this genetic material, even after several generations of cell divisions. In such embodiments, the modified plant, plant part, cell or protoplast may be referred to as a transgenic plant, plant part, cell or protoplast. Transiently transformed plant cells refer to cells which contain heterologous DNA or RNA, and are capable of expressing the trait conferred by the heterologous genetic material, without having fully incorporated that genetic material into the cell's DNA. Heterologous genetic material may be incorporated into nuclear or plastid (chloroplastic or mitochondrial) genomes as required to suit the application of the invention. In such embodiments, the modified plant, plant part, cell or protoplast may be referred to as a non-transgenic plant, plant part, cell or protoplast. Where plants are transformed with more than one polynucleotide it is envisaged that combinations of stable and transient transformations are possible.

Plants transduced with a polynucleotide or expression cassette or vector of the invention may be produced by standard techniques for the genetic manipulation of plants which are known in the art. DNA may be introduced into plant cells using any suitable technology including viral vector carrier transfer. Suitable viral vectors can be DNA or RNA viruses, including Bean yellow dwarf virus, Wheat dwarf virus, Cabbage leaf curl virus and Tobacco rattle virus.

The method as described herein may further comprise a step of expressing the RKD transcription factor or inducing expression of the RKD transcription factor. Inducing expression of RKD transcription factor in a plant may be achieved by an exposing the plant to an inducer. Suitable inducers include alcohol, tetracycline, dexamethasone, heat, cold, metals, pathogenesis related proteins. The method may further comprise the step of inducing gene editing to occur, for example by inducing components of the gene editing system to be expressed. Suitable gene editing systems are described elsewhere herein and may be selected from the list comprising of CRISPR-CAS9, CRISPR-CAS12a, TALENS, zinc-finger nucleases.

Suitably such a step of inducing expression of the RKD transcription factor may be applicable when the RKD transcription factor is operably linked to an inducible promoter. Suitably inducible promoters are described above. Suitably therefore this step may comprise contacting the plant, plant part, cell or protoplast with an effective concentration of an inducer. Suitably an effective concentration is a concentration sufficient to induce expression of the RKD transcription factor from the promoter. Suitably the inducer is capable of stimulating transcription from the inducible promoter, for example if the inducible promoter is an ethanol-inducible promoter, then the inducer used is ethanol.

Suitably the step of expressing the RKD transcription factor or inducing expression of the RKD transcription factor may comprise ectopically expressing the RKD transcription factor or inducing ectopic expression of the RKD transcription factor. Suitably in such embodiments the RKD transcription factor is expressed in a tissue of the plant, or plant part, or in a cell or protoplast thereof in which it is not usually expressed. Suitably the step may comprise ectopically expressing the RKD transcription factor or inducing ectopic expression of the RKD transcription factor in vegetative tissue of the plant, or plant part, or in a cell or protoplast thereof. Suitably the step may comprise ectopically expressing a reproductive RKD transcription factor or inducing ectopic expression of a reproductive RKD transcription factor in vegetative tissue of the plant, or plant part, or in a cell or protoplast thereof. Suitably therefore the methods of the invention are methods for ectopically expressing an RKD transcription factor in a plant, plant part, cell or protoplast thereof as explained hereinabove.

The method may further comprise a step of selecting a modified plant, plant part, cell or protoplast thereof having an increase in RKD transcription factor expression by:
Comparing the measured expression level to a reference expression level of the same RKD transcription factor in a reference plant, plant part, cell or protoplast thereof; and Selecting the modified plant, plant part, cell or protoplast if the measured expression level is increased relative to the reference expression level.

Suitable selection methods also include selection of modified plants, plant parts, cells or protoplasts via a selectable marker indicative of expression of the RKD transcription factor using for example fluorescence markers or resistance to herbicides as discussed above. Suitable selection methods also comprise identifying upregulation of histone methylation or trehalose production in modified plant, plant part, cell or protoplast thereof and selecting the plant, plant part, cell or protoplast on this basis. Suitably such plants, plant parts, cells or protoplasts will have a corresponding increase in RKD transcription factor expression.

The method may further comprise a step of regenerating a modified plant from the or each modified plant part, cell or protoplast. The step may comprise culturing the or each modified plant part, cell or protoplast. Culturing may include incubating the or each modified plant part, cell or protoplast in solid or liquid media culture under conditions conducive to regeneration. Such conditions may include for rice-16 h light/8 h dark with 200 μmol m⁻² s⁻¹ light intensity and temperature 28° C. daytime, 25° C. night, and for wheat 16 h light/8 h dark with 230 μmol m⁻² s⁻¹ light intensity and temperature 22° C. daytime, 20° C. night. Suitable conditions include any condition that allows the or each modified plant part, cell or protoplast to form a callus culture, and eventually to form a mature plant.

### A Method of Selecting a Plant

In a further aspect, there is provided a method of selecting a plant, plant part, cell or protoplast thereof comprising an increase in a yield-related trait relative to a reference plant, plant part, cell or protoplast thereof wherein the method comprises:
(a) Providing a plant, plant part, cell or protoplast thereof;
(b) Optionally mutagenizing the plant, plant part, cell or protoplast thereof;
(c) Measuring the expression level of at least one RKD transcription factor or an associated marker thereof in at least one cell of the plant, in the plant part, cell or protoplast thereof;
(d) Comparing the measured expression level to a reference expression level of the same RKD transcription factor or an associated marker thereof in a reference plant, plant part, cell or protoplast thereof; and
(e) Selecting the plant, plant part, cell or protoplast thereof if the measured expression level is increased relative to the reference expression level.

In one embodiment, the provided plant, plant part, cell or protoplast thereof may have been obtained by the methods as set out above. Therefore, in some embodiments of the method of selection, the plant, plant part, cell or protoplast thereof may have been modified by the methods of the first or second aspects by providing an RKD transcription factor, polynucleotide encoding an RKD transcription factor or gene editing system to the plant, plant part, cell or protoplast thereof.

Alternatively, the plant, plant part, cell or protoplast thereof may undergo a step of mutagenesis, suitably random mutagenesis. Suitably, in this embodiment, the plant, plant, plant part, cell or protoplast thereof has not been obtained by the methods as set out above. Suitably in this embodiment the plant, plant part, cell or protoplast provided at step (a) is unmodified.

Mutagenizing as used herein may refer to introducing mutations into a polynucleotide or a protein. Mutagenizing may include exposing the plant to radiation, chemicals and/or enzymes or any suitable technique known in the art to introduce desired mutants or traits into the plant. Radiation may include exposing the plant to ionizing or non-ionizing radiation. Suitably, the plant may be mutated by exposure to x-rays or y-radiation. Chemical mutagens may include alkylating agents, suitably ethyl methanesulfonate or nitroso compounds. Exposing plants to enzymes such as restriction enzymes can introduce double strand breaks in the DNA. Suitable restriction enzymes for mutating plants include bacterial endonucleases such as Fok1 or Cas9.

In a further alternative, the plant, plant part, cell or protoplast thereof may have undergone spontaneous mutagenesis. Suitably in this embodiment, the plant, plant, plant part, cell or protoplast thereof has not been obtained by the methods as set out above, nor has step (b) of mutagenizing been carried out. In this embodiment, the plant, plant part, cell or protoplast of step (a) may comprise a mutation which has occurred naturally and which increases the expression of an RKD transcription factor. Suitably, therefore the method may be used to screen populations of plants, plant parts, cells or protoplasts for mutants comprising an increase in a yield-related trait.

Measuring the expression level of at least one RKD transcription factor or an associated marker thereof in at least one cell of the plant, in the plant part, cell or protoplast thereof may comprise measuring the direct expression level of the RKD transcription factor. This may be achieved by methods known in the art for monitoring gene expression such as RT-PCR or Q-PCR, or by methods known in the art for monitoring protein expression such as western blotting or ELISA.

Alternatively, measuring the expression level of at least one RKD transcription factor or an associated marker thereof in at least one cell of the plant, in the plant part, cell or protoplast may comprise measuring the indirect expression level of the RKD transcription factor by measuring the level of an associated marker. This may be achieved by monitoring the expression level of a marker which is indicative of expression of the RKD transcription factor. Suitable associated markers may be for example markers introduced into the plant, plant part, cell or protoplast thereof together with the modification when providing an RKD transcription factor, polynucleotide encoding an RKD transcription factor or gene editing system to the plant, plant part, cell or protoplast thereof. Such suitable associated markers may be fluorescence markers as described elsewhere herein. These associated markers may be measured by any means known in the art which can measure fluorescence, such as fluorometers, spectrofluorometers and confocal microscopy. Other suitable associated markers may be for example endogenous markers which change as a result of providing an RKD transcription factor, polynucleotide encoding an RKD transcription factor or gene editing system to the plant, plant part, cell or protoplast thereof. Such suitable markers may comprise downstream effects caused by providing an RKD transcription factor, polynucleotide encoding an RKD transcription factor or gene editing system to the plant, plant part, cell or protoplast thereof, for example an upregulation in histone methylation or increased trehalose production in the modified plant, plant part, cell or protoplast thereof.

These associated markers may be measured by any means known in the art, methylation may be measured by chromatin immunoprecipitation assays with sequencing (ChIP-Seq), trehalose production may be measured by ChiP-Seq and/or ChIP-PCR and/or chemical analyses such as HPLC.

Suitably measurement of the expression level of at least one RKD transcription factor or associated marker provides a ‘measured expression level’.

Comparing the measured expression level to a reference expression level of the same RKD transcription factor or an associated marker thereof in a reference plant, plant part, cell or protoplast thereof suitably comprises making the same measurement made in step (b) in a reference plant. Suitably, this step further comprises comparing the value obtained in the modified plant, (the measured expression level), to the value obtained in the reference plant, (the reference expression level). Suitably this comparison may be automated.

Suitably the selection step (e) comprises selecting the modified plant, part, cell or protoplast thereof if the measured expression level in the modified plant is increased relative to the reference expression level of an equivalent unmodified plant. Suitably, for example, this step may comprise selecting the modified plant, part, cell or protoplast thereof if the measured level of gene or protein expression of an RKD transcription factor, is increased relative to the level of expression of the same RKD transcription factor gene or protein in a reference plant, part, cell or protoplast thereof. Suitably, for example, this step may comprise selecting the modified plant, part, cell or protoplast thereof if the measured level of histone methylation is increased relative to the level of histone methylation of the same histone in a reference plant, part, cell or protoplast thereof. Suitably, for example, this step may comprise selecting the modified plant, part, cell or protoplast thereof if the measured level of trehalose, is increased relative to the level of trehalose in a reference plant, part, cell or protoplast thereof.

Suitably the method of selecting the plant, plant part, cell or protoplasts provides a means of selecting those plants, plant parts, cells or protoplasts that are likely to have an increase in a yield-related trait due to an increase in expression of an RKD transcription factor.

Suitably the method may further comprise a step of cultivating the plant, plant part, cell or protoplast thereof to maturity. Suitably the method may further comprise a step of harvesting, sowing, germinating or cultivating seeds obtained from the selected plant.

### Plant Breeding and Hybrid Production

In a further aspect, there is provided herein a method of plant breeding and/or plant improvement comprising combining the genetic material of a first plant obtained by a method of the invention with the genetic material of a second plant.

Suitably the first plant is a modified plant. Suitably the first plant is a plant modified to have an increase in expression of an RKD transcription factor.

In one embodiment, the second plant may be a plant obtained by a method of the invention or may be another plant differing in genotype. Suitably therefore in some embodiments, the second plant may also be a modified plant, and may also be a plant modified to have an increase in expression of an RKD transcription factor. In other cases the second plant may be an unmodified plant, effectively a reference plant. Suitably combining the genetic material of the two plants may comprise crossing the first plant obtained by the method of the invention with a second plant.

In one embodiment, progeny are produced which comprise increased expression of an RKD transcription factor thereof relative to a reference plant. Suitably the progeny may be termed F1 progeny.

In one embodiment, the method is a method of plant breeding and/or plant improvement such that the plant progeny will have an increase in a yield-related trait relative to a reference plant.

In a further aspect, there is provided herein a method of producing a hybrid seed comprising crossing a first plant obtained by a method of the invention with a second plant; and obtaining a seed.

Suitably the first plant is a modified plant. Suitably the first plant is a plant modified to have an increase in expression of an RKD transcription factor.

In one embodiment, the second plant may be a plant obtained by a method of the invention or may be another plant differing in genotype. Suitably therefore in some embodiments, the second plant may also be a modified plant, and may also be a plant modified to have an increase in expression of an RKD transcription factor. In other cases the second plant may be an unmodified plant, effectively a reference plant.

In one embodiment, the seed comprises a genotype which confers an increased expression of an RKD transcription factor relative to a reference plant.

In a further aspect of the present invention, there is provided a hybrid seed obtained by the method of the ninth aspect and/or a hybrid plant generated from the hybrid seed.

### BRIEF DESCRIPTION OF THE FIGURES

FIG. 1: A picture of Aegilops tauschii and Triticum aestivum, showing the difference in plant architectures. Aegilops tauschii is dwarf and bushy and Triticum aestivum is upright and has fewer tillers.

FIG. 2(a): Tiller number in Ae. tauschii crops compared with AetRKD1 relative expression levels, AetRKD1 correlates with tiller number. (b) Major loci for genome wide association with tiller number, the box indicates the location of the GWAS peak associated with high tiller number in chromosome 7.

FIG. 3: Heatmap showing RKD transcription factor expression patterns in different tissues of Triticum aestivum.

FIG. 4: Phylogenetic tree showing RKD transcription factor homologues in other plant species. TaRKDs are divided to two subclasses, reproductive and vegetative. TaRKD1 homologues are specifically expressed in embryonic cells and meristem tissues.

FIG. 5: Comparison of RKD1 expression in hexaploidy wheats. TaRKD1 is strongly expressed in immature embryos, but not expressed in leaf blades and axillary meristems of T. aestivum.

FIG. 6: In Ae tauschii, AeRKD1 was highly accumulated in immature embryo and in axillary meristems of some tauschii accessions (arrows). Tauschii accessions with high, AeRKD1 expression have a greater number of tillers than the other accessions.

FIG. 7: (a) TaRKD1 ectopic overexpression in wheat increases effective tiller number (b) shows tiller number in TaRKD1-OX clones #1.4_5, #1.4_2, #2.28_5 and 2.28_11 compared to wild type tiller number.

FIG. 8(a): Monitoring axil number according to developmental stages in wild type and modified wheat with overexpression of RKD1; Wild type and TaRKD1-OX #1.4 and #2.28 clone tiller number were monitored at day 21, 28 and 32 days after sowing, (b) Number of cells undergoing division is increased in overexpression clones TaRKD1-OX.

FIG. 9: Numerical comparison of axil number between WT and transgenic lines of wheat from FIG. 8a (RKD1 overexpression) Axil number of several plants were counted. By two weeks, axil number and rate of axils are both increased in transgenic plants compared to WT.

FIG. 10: (a) Number of spikelets was increased by ectopic overexpression of TaRKD1 in Wild type and TaRKD1-OX #1.4 and #2.28 lines, the left microscopy images show immature inflorescences, (b) Similar differences are shown in SEM analysis, WT had on average 17 to 19 spikelet meristems, but transgenics had 21 to 23 meristem spikelets.

FIG. 11(a): Average spikelet length in TaRKD1 overexpressing clones #1.4 and #2.28 compared to wild type, length is increased in overexpression lines, (b) and (c) and (d) Average number of spikelets and grains per spike in TaRKD1 overexpressing clones #1.4 and #2.28 compared to wild type and a null segregant, number of spikelets and grains per spike is increased in overexpression lines, (e) Seed weight per plant in grams and (f) weight per 1,000 grains in grams is also increased in TaRKD1-OX lines.

FIG. 12: (a) RNAseq analysis of immature embryo tissues shows enrichment of chromatin accessibility in upregulated differentially expressed genes (DEGs) in TaRKD1-7D OX lines compared to wild type, (b) RNAseq analysis of immature embryo tissues shows enrichment of chromatin accessibility in upregulated differentially expressed genes (DEGs) in lines with ectopic expression of RKD1.

FIGS. 13(a) and (b): Co-expression analysis for upregulated DEGs and networks. Histone modification factors and cell cycling factors were enriched and DNA methylation factors and transcription factors were also abundant in TaRKD1 OX lines. Cell cycle and histone H3K27me3 methylation are connected in co-expression network. Histone H4 genes in top outdegree nodes in co-expression network are upregulated in axillary meristem. Among top outdegree nodes in co-expression network, several Histone H4 genes were enriched. (c) RNA in situ hybridization was performed showing Histone H4 signal was increased, and the signal was more spread in the transgenic plants.

FIG. 14: Enrichment of TaRKD1 transcription factor binding in promoter regions. DAPseq and ChIPseq were performed to determine regulatory mechanisms of TaRKD1 in meristem initiations using TaRKD1 antibody. There is an enrichment of TaRKD1 binding in promoter regions. ChIPseq signals were more abundant than DAPseq signal.

FIG. 15: Venn diagram of up and down regulated DEGs with ChiPseq_DAPseq_10 kb. TaRKD1 transcription factor binds promoters of upregulated meristematic determinacy factors such as trehalose phosphate synthase.

FIG. 16: A phylogeny tree showing reproductive OsRKD3 in rice. OsRKD3 is grouped with wheat reproductive RKD transcription factors.

FIG. 17a-e: OsRKD3 overexpression in rice increases grain yield via changes of tiller number and panicle branches. (a) The pictures show increased tiller number in OsRKD3-OX lines compared to wild type. Numerical analysis shows a significant increase in (b) tiller number, (c) seeds per panicle, (d) seeds per plant and (e) panicle branching in OsRKD3-OX lines compared to wild type.

FIGS. 18(a) and (b): Arabidopsis homologue, AtRKD4-overexpression (AtRKD4-OX) in Arabidopsis plants increased (a) axil number and (b) increased tiller number as compared to wild type.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

### Definitions and General Points

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.-in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

The terms “comprising”, “comprises” and “comprised of” as used herein are synonymous with “including”, “includes” or “containing”, “contains”, and are inclusive or open-ended and do not exclude additional, non-recited features, elements or method steps.

The recitation of numerical ranges by endpoints includes all numbers and fractions subsumed within the respective ranges, as well as the recited endpoints.

A “variant” or “functional variant” in the context of the present invention is a variant of a reference polynucleotide sequence and/or transcription factor that retains the ability to function in the same way as the reference polynucleotide sequence and/or transcription factor. Alternative terms for such variants include “biological equivalents” or “equivalents”.

An “active fragment” in the context of the present invention may refer to a minimal part or subunit of a polynucleotide and/or transcription factor of the present invention capable of functioning in the same way as the reference polynucleotide sequence and/or transcription factor.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215:403-10), such as the “Blast 2 sequences” algorithm described by Tatusova and Madden 1999 (FEMS Microbiol Lett 174:247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication deficient as they have lost the ability to propagate in a given cell since viral genes essential for replication have been eliminated from the viral vector. However, some viral vectors can also be adapted to replicate specifically in a given cell. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “cell” as used herein may refer to any eukaryotic cell, preferably a plant cell. Plant cells include any cell in the shoot system and/or root system. Cells may include any of: parenchyma cells, collenchyma cells, sclerenchyma cells, xylem cells, phloem cells, meristematic cells and epidermal cells. A cell may also include a protoplast. A protoplast is a plant cell which lacks a cell wall.

The term “polynucleotide” as used herein may refer to a compound comprised of several nucleotides joined together. The nucleotides may be joined by covalent bonds. Examples of polynucleotides include DNA and RNA.

Flowering plants comprise two classes ‘monocots’ and ‘dicots’ that are differentiated by the number of cotyledons present in the seed embryo. Monocots have one cotyledon and dicots have two cotyledons. Monocots comprise most bulbing plants, grasses and grains, such as agapanthus, asparagus, bamboo, bananas, corn, daffodils, garlic, ginger, grass, lilies, onions, orchids, rice, sugarcane, tulips, and wheat. Dicots comprise legumes, cabbage family plants and other commercially important plants such as fruit trees.

The terms “commercially important” and “economic significance” as used herein may refer to any plants that contribute to the stability of the human and animal food supply chain. Such plants include those that are important to economic stability and trade. Such plants typically include crop plants, examples of commercially important plants such as crops are given in the relevant section hereinabove.

A “phenotype” as used in the context of the present invention refers to the physical characteristics of a plant. This characteristic may be a genetically or environmentally determined.

A “trait” as used herein refers to a single or multiple features of a plant. Such traits include ‘tillering’ which refers to the production of side shoots on the plant. Other traits include ‘inflorescence’ which may refer to the process of flowering, the arrangement of flowers on a branch system and/or a system of branches. In the context of the present invention, a “yield-related trait” is any trait that determines or is associated with the amount of biomass that can be harvested from a given plant, further explanation of this term is given in the relevant section hereinabove.

The term “genotype” refers to the genetic makeup of an organism. Genotype may refer to one gene, a set of genes or the complete set of genetic material of an organism.

The term “homologous” as used herein may refer to an RKD transcription factor and/or a polynucleotide encoding an RKD transcription factor that originates from the same plant or plant species as that being modified.

The term “improving plants” as used herein, may refer to modifying a plant to have a desired characteristic or trait. In accordance with the invention the desired characteristic or trait is a yield related trait. In one context, an improved plant may have an increase in a yield-related trait, for example increased tillering, in another, a plant may have increased seed production, as compared to an unmodified reference plant.

A “hybrid plant” or “hybrid seed” as used in the context of the present invention may refer to the offspring (i.e. progeny) of cross breeding one plant variety with another plant variety. The hybrid may comprise the characteristics of both parent plant varieties. A hybrid may be an F1 or F2 hybrid. An F1 hybrid is the first-generation plant obtained from direct crossing of two parental varieties. An F2 hybrid is the offspring of the F1 hybrid.

“Cross breeding” or “crossing” as used herein in the context of the present invention may refer to producing a plant by mating or hybridizing two different species, breeds, or varieties to produce a new variety of plant.

A “transcription factor” as used herein refers to any protein that regulates the transcription of genes. A transcription factor binds to regions of DNA known as transcription factor binding sites. A transcription factor may activate gene expression or may repress gene expression.

The term “expression level” as used in the context of the present invention refers to the quantity of an expression product of a nucleotide sequence (e.g. mRNA or protein). Expression levels can be measured by various conventional means, such as quantitative PCR, by antibody-based assays, e.g. a Western Blot or an ELISA assay, for instance to evaluate whether expression of the expression product is achieved. Expression of the expression product may also be measured in a bioassay that detects an enzymatic or biological activity of the gene product. The expression level is compared to a reference expression level. A reference or control expression level may refer to the baseline level of expression in a reference plant. The reference expression level may be the average expression level of a plurality of reference plants.

A “modified plant” as used in the context of the present invention may refer to the ‘engineering’ or ‘altering’ of any plant, plant part, plant cell or protoplast to change the properties of the plant. For example, a modified plant, plant part, cell or protoplast includes providing a RKD transcription factor or a variant or active fragment thereof, a polynucleotide encoding said RKD transcription factor, variant or active fragment thereof or a gene editing system for modifying an endogenous nucleic acid sequence encoding an RKD transcription factor or a regulatory sequence controlling expression thereof to said plant, plant part, cell or protoplast. A modified plant, plant part, cell or protoplast may be generated for example by genetic modification, genetic transformation, or gene editing and refers to plants, parts, cells or protoplast thereof which have an increase in a yield-related trait compared to an unmodified reference plant, part, cell or protoplast.

The term “reference plant” as used herein refers to any equivalent plant, plant part, cell or protoplast to the modified plant, part, cell or protoplast that has not been modified. The reference plant can be a control plant. The reference plant may be a wild type plant of the same species as the modified plant. A reference plant may be a plurality of plants, parts, cells or protoplasts.

“Associated marker” as used herein may refer to any genetic, epigenetic or phenotypic factor associated with providing an RKD transcription factor, polynucleotide encoding a RKD transcription factor or a gene-editing system to a plant, plant part, cell or protoplast. An associated marker may include a fluorescent marker or herbicide resistance marker as provided as part of an expression cassette, vector or gene editing system. An example of an associated marker may also include increased biological effects associated with RKD transcription factors, such as increased chromatin remodeling, increased histone H4 and/or increase H3K27 methylation.

### EXAMPLES

### Materials and Methods

### Plant Materials and Growth Conditions

In this study, we used bread wheat (Triticum aestivum), Durum wheat (Triticum durum), goat grass (Aegilops tauschii) and synthetic hexaploid wheat. The accessions of A. tauschii were obtained from the John Innes Centre (JIC) germplasm resource (Norwich, UK) and T. aestivum, T. durum and synthetic hexaploid wheat were got from National Institute of Agricultural Botany (NIAB, Cambridge, UK). Wheat seeds were stratified on the moist paper at 4° C. for 2 days, transferred to the pots (diameter 28 cm) containing peat-based soil, and grown to flowering. All wheat plants were grown at 22/20° C. day/night in a 16/8 h light/dark cycle under light intensity of 150 μE m⁻²s⁻¹. Axillary meristems and leaves were collected at 3 weeks after sowing, and immature embryos were manually dissected from developing wheat seeds at 12-18 days after anthesis.

Indonesian black rice seeds (Oryza sativa L. Cempo Ireng) were obtained from the Research Institute for Agriculture Technology (Yogyakarta, Indonesia) and Nipponbare seeds were obtained from NIAB (Cambridge, UK). Mature seeds of rice were dehusked, sterilized, washed, planted on the half-strength Murashige and Skoog (MS) medium containing 3% sucrose, and grown for one week at 28° C. under constitutive light. The grown seedlings were transferred to the pots (diameter 28 cm) containing peat-based soil and grown at 28/25° C. day/night in a 16/8 h light/dark cycle under light intensity of 230 μE m⁻²s⁻¹ until 8 weeks. Eight weeks after transplantation, short-day treatment (12 h light/12 h dark) was performed for two weeks to induce floral transition of the rice and the treated plants were grown to ripening stages under 16 h day/8 h night conditions.

### Vector Construction and Agrobacterium-Mediated Transformation

For construction of the inducible-TaRKD1-7D, we designed codon-optimized full-length CDS construct (synTaRKD1) that was chemically synthesized (IDT, Leuven, BE). The synTaRKD1 fragment was introduced into a pDONR207 vector (Invitrogen, USA) via BP recombination and subcloned by LR recombination in the two-component chemically inducible vector pERV1 (Valdivia et al., 2013). The inducible-OsRKD3 transgenics were generated in the cv Cempo ireng background. We designed a full-length codon-optimised OsRKD3 (synOsRKD3) and chemically synthesised (IDT, Leuven, BE). For generation of the inducible-AtRKD4, we designed full-length CDS of AtRKD4 (synAtRKD4), a codon-optimized for rice and chemically synthesized (IDT, Leuven, BE). The synthesized synOsRKD3 and synAtRKD4 were introduced into a pDONR207 vector (Invitrogen, USA) via BP recombination and subcloned by LR recombination in the two-component chemically inducible vector pTA7200 (Aoyama and Chua, 1997) and pERV1 (Valdivia et al., 2013) respectively. The pERV1-synTaRKD1, pTA7002-synOsRKD3 and pERV1-synAtRKD4 were fully sequenced and transformed into Agrobacterium tumefaciens EHA105. The pERV1-synTaRKD1 vector, thereafter named indTaRKD1, was fully sequenced and the construct was introduced into Agrobacterium tumefaciens EHA105. Wheat and rice transformation was performed by Agrobacterium-mediated co-cultivation method as previously described (Lee et al., 1999, Toki et al., 2006, Susanto et al., 2020).

### Phylogenetic Tree and Protein Alignments

RKD protein sequences were obtained from NCBI, TAIR, MSU TIGR and Ensemble database. The alignment of RKD proteins was computed using MAFFT in Jalview (Waterhouse et al., 2009). Phylogenetic tree was constructed using MEGA X (Kumar et al., 2016) using the minimum evolution method (Rzhetsky and Nei, 1992). The tree was drawn to scale, with branch lengths in the same units as those of the evolutionary distances used to infer the phylogenetic tree. The evolutionary distances were computed using the Poisson correction method (Zuckerkandl and Pauling, 1965) and are in the units of the number of amino acid substitutions per site. The ME tree was searched using the Close-Neighbour-Interchange (CNI) algorithm (Nei and Kumar, 2000) at a search level of 1. The Neighbour-joining algorithm (Saitou and Nei, 1987) was used to generate the initial tree. All ambiguous positions were removed for each sequence pair (pairwise deletion option). There were a total of 1486 positions in the final dataset. Evolutionary analyses were conducted in MEGA X (Kumar et al., 2018; Stecher et al., 2020).

### Preparation of RNA Probes

To generate RNA probes for Histone H4, we selected a highly conserved region of 190 bp of wheat Histone H4 sequences (Drea et al., 2005) and. For in vitro transcription, T7 promoter and T3 promoter were added to each end of the selected Histone H4 and we chemically synthesized the designed sequence (IDT, Leuven, BE). A histone H4 fragment was transcribed by T7 or T3 RNA polymerase (Roche) for 2 hours at 37° C., labelled by DIG-UTP (Roche) during in vitro transcription and treated by stop solution (0.1 Units·μL⁻¹ DNase (Ambion, USA), 10 mM Tris-Cl, 10 mM MgCl₂, 50 mM NaCl, pH 7.5) for 30 minutes at 37° C. The synthesized RNA probes were precipitated in precipitation solution (0.8 M LiCl, 0.3 M NaOAc, 200 μg·mL⁻¹ tRNA (Sigma, St. Louis, MO), 66.7% (v/v) ethanol, pH 5.2) overnight at −20° C., centrifuged for 10 minutes at 13000 rpm at 4° C., washed using 70% ethanol, spun down for 10 minutes at 13000 rpm at 4° C., discarded supernatant, air dried and dissolved in DEPC-treated distilled water. The dissolved RNA probes were hydrolysed in carbonate buffer (40 mM NaHCO₃, 60 mM Na₂HCO₃, pH 10.2) for 30 minutes at 60° C., precipitated in precipitation solution (10% (v/v) acetic acid, 90 mM NaOAc, 66.7% ethanol, pH 5.2) for 2 hours at −80° C., spun down for 10 minutes at 13000 rpm at 4° C., washed using 70% ethanol, spun down for 10 minutes at 13000 rpm at 4° C., discarded supernatant, air dried and dissolved in DEPC-treated distilled water.

### RNA In Situ Hybridization in Axillary Meristems

To avoid contaminations of RNase, glassware was baked at 180° C. for 8 hours, plasticware was treated by 0.1 N NaOH (Sigma, St. Louis, MO) overnight at 37° C. and solutions were treated by 0.1% diethyl pyrocarbonate (DEPC, Sigma, St. Louis, MO). For RNA in situ hybridization in developing axils of WT and indTaRKD1-7D, the axils of the basal leaves of the main shoot were submerged in 4% paraformaldehyde (PFA, Agar Scientific, Essex, UK) and vacuum infiltrated three times for 10 minutes. The fixative was then replaced, and the sample incubated overnight at 4° C. After fixation, the samples were washed using phosphate buffered saline (PBS; 10 mM sodium phosphate and 150 mM NaCl, pH 7.4) with 0.1% DEPC, dehydrated via a graded ethanol series and embedded in Paraplast Plus (Sigma, St. Louis, MO). Sections were longitudinally cut at 10 μm and mounted on Superfrost Plus slides (Thermo Scientific, UK). The mounted samples were deparaffinized using Histoclear (National Diagnostics, Hull, UK), rehydrated through an ethanol series, treated by Pronase (Merck, Darmstadt, Germany) for 10 minutes at 37° C., fixed by 4% PFA for 10 minutes at room temperature, acetylated by acetic anhydride (Sigma, St. Louis, MO) in 0.1 M triethanolamine (Sigma, St. Louis, MO) for 10 minutes at room temperature and re-dehydrated by an ethanol series. To prepare RNA probe hybridization solution, subsequently, hybridization buffer (10 mM Tris-Cl, 0.3 M NaCl, 10 mM NaPO₄, 5 mM EDTA, 1.25 mg mL-1 tRNA (Sigma, St. Louis, MO), 1.25×Denhardt's (Sigma, St. Louis, MO), 12.5% dextran sulphate (AP Biotech), 50% deionized formamide (Sigma, St. Louis, MO), pH 6.8) was mixed by vortexing, spun down, and left at room temperature. RNA probe solution was prepared with mixing between RNA probe and deionized formamide in 1:1 ratio, heat the mix solution for 2 minutes at 80° C., spun down and cool on ice. The probe solution was mixed with the hybridization buffer in a 4:1 buffer: probe ratio, vortexed, spun down and left at room temperature. To prevent RNA probes from adhering to the coverslip, that was treated by Sigmacote (Sigma, St. Louis, MO) for 10 seconds at room temperature, washed by absolute ethanol and dried in fume hood before starting hybridization procedures. 50-100 μL of RNA probe hybridization solution was added on the slide, covered by Sigmacote-treated coverslip, incubated in humid chamber overnight at 45° C. The slides were dipped in 2×SSC buffer (150 mM NaCl, 15 mM NaOAc), agitated gently until the coverslips were fell off, moved to a new 2×SSC buffer, incubated for 30 minutes at 45° C., moved once more to a new 2×SSC buffer, incubated for 90 minutes at 45° C., washed twice using NTE buffer (10 mM Tris-Cl, 0.5 M NaCl, 1 mM EDTA, PH 7.5) for 5 minutes at 37° C., treated by RNase buffer (20 μg·mL⁻¹ RNase A (Sigma, St. Louis, MO) in NTE buffer) for 30 minutes at 37° C., washed three times using NTE buffer for 5 minutes at 37° C., incubated in 0.5×SSC buffer for 1 hour at 45° C. and washed using PBS for 5 minutes at room temperature. The washed slides were incubated for 5 minutes in DIG Buffer I (100 mM Tris-Cl, 150 mM NaCl, pH 7.5) at room temperature, blocked for 30 minutes in DIG Buffer II (0.5% (w/v) blocking reagent (Boehringer, Germany) in DIG buffer I) at room temperature, transferred to DIG Buffer III (0.1% (w/v) BSA, 0.3% (v/v) Triton X-100 (Sigma, St. Louis, MO) in DIG Buffer I), incubated for 30 minutes in DIG Buffer III at room temperature, transferred to DIG Buffer IV (1:3000 anti-digoxigenin-AP (Roche) in DIG Buffer III), incubated for 90 minutes in DIG Buffer IV at room temperature, washed four times using DIG Buffer III for 20 minutes at room temperature, washed using DIG Buffer I for 5 minutes at room temperature, washed using DIG Buffer V (100 mM Tris-Cl, 100 mM NaCl, 50 mM MgCl₂, pH 9.5) for 5 minutes at room temperature and incubated for 6 to 36 hours in Buffer VI (75 mg mL-1 nitroblue tetrazolium (NBT, Sigma, Pooler, UK), 50 mg·mL⁻¹ 5-bromo-4-chloro-3-indolylphosphate (BCIP, Sigma, Pooler, UK), 24 mg·mL⁻¹ Levamisole (Sigma, Pooler, UK) in DIG Buffer V) under dark conditions at room temperature, incubated in TE buffer (10 mM Tris-Cl, 1 mM EDTA, PH 7.5) for 10 minutes at room temperature, washed for 2 minutes in distilled water, dehydrated through an ethanol series, dried completely, mounted in Entellan (Merck, UK) and covered by coverslip.

### Toluidine Blue Staining of Axillary Meristem Sections

For toluidine bule staining of developing axils in WT and indTaRKD1-7D, sections of developing axils were longitudinally cut at 10 μm and mounted on Superfrost Plus slides (Thermo Scientific, UK). The mounted samples were deparaffinized using Histoclear (National Diagnostics, Hull, UK), rehydrated through an ethanol series, stained with Toluidine Blue solution for 2 minutes at room temperature, washed for 2 minutes in distilled water, dehydrated via an ethanol series, dried completely, mounted in Entellan (Merck, UK), and covered by coverslip.

### Antisera Preparation and Immunolocalization

Polyclonal antisera were raised in rabbit against synthetic peptides for TaRKD1-7D (CFKENYKRRRAAASVN SEQ ID NO: 40 and ARWPHRKMKSLRSLI SEQ ID NO: 41) (Eurogentec, Liege, BE). The raised antisera were affinity purified with peptides (CFKENYKRRRAAASVN SEQ ID NO: 40 and ARWPHRKMKSLRSLI SEQ ID NO: 41) using a Sulpholink coupling gel system (Pierce, Rockford, IL). For immunolocalization of TaRKD1-7D in axillary meristem, sections were longitudinally cut at 10 μm and mounted on Superfrost Plus slides (Thermo Scientific, UK). The mounted samples were deparaffinized using Histoclear (National Diagnostics, Hull, UK), rehydrated through an ethanol series, and blocked in 0.1% bovine serum albumin (BSA) in phosphate buffered saline (PBS; 10 mM sodium phosphate and 150 mM NaCl, pH 7.4) for 30 mins at room temperature and incubated overnight with purified anti-TaRKD1-7D (diluted 1:20) antibody at 4° C. The immunoreactions were performed using goat anti-rabbit alkaline phosphatase-conjugated secondary antibody (Sigma, St. Louis, USA; diluted 1:100) and nitroblue tetrazolium/5-bromo-4-chloro-3-indolylphosphate (NBT/BCIP; Sigma, Pooler, UK) was used as detection substrate. The stained slides by NBT/BCIP were mounted in Entellan (Merck, UK).

### Estradiol and Dexamethasone Treatment and Collection of the Treated Leaf Samples

Leaf segments or immature embryos excised from the caryopsis 12-18 days after anthesis were soaked in 40 UM of B-estradiol (Sigma, UK) or 20 μM Dexamethasone solution containing 0.01% (w/v) Tween-20 and incubated at 22° C. for 3 hours. As a mock control, tissues were also incubated in 0.02% (w/v) DMSO solution containing 0.01% Tween-20. After treatment, the solution was removed from the samples using a paper towel. The collected samples were flash frozen in liquid nitrogen and then stored at −80° C.

### RNA Extraction, Library Preparation and Sequencing

Total RNAs were extracted from the immature embryos or leaves with Qiagen RNeasy Plant Mini kit (Qiagen, Germany) as manufacturer's protocols. RNAs for sequencing were extracted using Direct-zol RNA miniprep kit (ZYMO Research, Cambridge). The extracted RNAs were quantified using Qubit fluorometer (Invitrogen, UK) and qualities of those were also checked through Bioanalyzer 2100 (Agilent, UK). Total RNA libraries were prepared using the TruSeq RNA Sample Preparation Kit (Illumina, UK) and sequenced in pair-end 150 bases mode an Illumina NexSeq platform at the Novogene.

DAP-Seq Analyses on Triticum aestivum Genomic DNA

For construction of HaloTag fused with TaRKD1-7D, pDONR207-synTaRKD1-7D vector was cloned into pIX-HALO in vitro expression vector via LR recombination, transformed into the BL21DE3 codon plus expression strain (Stratagene, USA), protein was expressed as the manufacturer's manual. Genomic DNA libraries were prepared according to Bartlett et al (2017), and genomic DNA was extracted from wheat leaves using Plant DNeasy kit (QIAGEN, Germany) as manufacturer's recommendations. 20 μg of genomic DNA was diluted in EB (10 mM Tris-HCl, pH 8.5) and sonicated to 200 bp fragments in a covaris S2 sonicator. DNA was purified using AmpureXP beads at a 2:1 bead: DNA ratio. Samples were then end repaired using the End-It kit (Lucigen) and cleaned using Qiaquick PCR purification (Qiagen) according to the manufacturer's recommendations. Purified samples were A-tailed using Klenow 3-5′ exo- for 30 min at RT and then purified using Qiaquick PCR purification as described above. Purified samples were then ligated overnight with a truncated Illumina Y-adapter as described in Bartlett et al. Libraries were purified by bead cleaning using a 1:1 bead: DNA ratio, eluted from the beads in 30 μL of EB, and quantified with the Qubit HS fluorometric assay. A quantity of 20 μl of purified GST-ARF protein (5-20 μg) was diluted in 400 μL of 1×PBS containing 25 μL of washed MagneGST beads (Promega) for 1 h at RT on a rotator to bind the protein to the beads. In addition to the GST-TaRKD1-7D samples, a negative control GST-HALO sample was performed using protein expressed in the TNT wheatgerm expression system (Promega). Beads were washed four times in 1×PBS+NP40 (0.005%) and resuspended in 100 μL of 1×PBS. One microgram of genomic DNA library (from ear was diluted to a final volume of 60 μl in 1×PBS and added to the protein bound beads. Protein bound beads and gDNA were rotated for 1 h at RT. Beads were washed four times in 1×PBS+NP40, followed by two washes with 1×PBS. Beads were transferred to a new tube and DNA was recovered by resuspending in 25 μL EB and boiling for 10 min at 98° C. Eluted samples were enriched and tagged with dual indexed multiplexing barcodes by performing 20 cycles of PCR in a 50 μL reaction. Samples were pooled and sequenced on a NExtSeq500 with 75 bp single end reads.

cDNA Synthesis and RT-PCR

Total RNA was treated with Ambion® TURBO DNase kit (Life technologies, USA) as manufacturer's protocols. DNase-treated RNAs were used for cDNA synthesis using first strand cDNA synthesis kit (ThermoFisher, UK) according to manufacturer's protocols. Semi-quantitative RT-PCR was performed using templates as the synthesized cDNAs. TaGAPDH was used for data normalization. For Quantitative real-time RT-PCR (qRT-PCR) the optimal number of cycles was determined for each gene. PCR cycling conditions included denaturing at 95° C. for 10 s, annealing at 57° C. for 20 s and extension at 72° C. for 30 s, using a Bio-rad qRT-PCR machine (Bio-Rad, UK). Changes in expression levels were calculated via the ΔΔCt method. To ensure primer specificity, qRT-PCR was done when the melting curve showed a single peak.

### RNA-Seq Library Construction and Data Analysis

RNAs for sequencing were extracted using the Direct-zol RNA miniprep kit (ZYMO Research, Cambridge). The extracted RNAs were quantified using Qubit HS kit (Invitrogen, UK) and quality checked using a Bioanalyzer 2100 (Agilent, UK). Total RNA libraries were prepared using the TruSeq RNA Sample Preparation Kit (Illumina, UK) and sequenced in paired-end 150 base mode an Illumina sequencing platform.

For the RNA-seq analysis in rice, sequencing reads were pre-processed using Trimmomatic v0.36 (Bolger et al., 2014) to remove reads with either low quality scores, irregular GC content, short length or sequencing adapters present. Trimmed reads were then quality checked using FastQC v0.11.5 (Andrews et al., 2015) and mapped to the Oryza sativa ssp. Japonica genome (release MSU 7.0) using HISAT2 v2.1.0 (Kim et al., 2015). Alignments were sorted using Samtools v0.1.19 (Li et al., 2009), counted using LiBiNorm v2.0 (Dyer et al., 2019) and then imported into R Studio (version 1.2.5033). Libraries were then normalised using DESeq2 v1.24 (Love et al., 2014) and significant differentially expressed genes (DEGs) were calculated using the combined criteria; p.adj<0.001, IfcThreshold>1. DEGs were then tested for GO term enrichment with AgriGO v2.0 (Tian et al., 2017) using a Fisher's exact test under the following criteria; FDR under dependency<0. Motif enrichment analysis was carried out using HOMER version 4.11 (Heinz et al., 2010) under default parameters and with an equal number of random gene flanking sequences as a background. De-novo motif enrichment results were used to select an appropriate transcription factor class implicated in the network. RKD binding motifs in their TSS flanking sequence were identified with HOMER and selected for co-expression network construction. Coexpression values for expressed genes were imported into Cytoscape version 3.6.1 (Shannon et al., 2003) and were visualized under a perfuse force directed layout according to MR Rank. Annotation and arrangement of nodes was carried out using Cytoscape and Adobe Illustrator (v24.0.2).

For the RNA-seq analysis in wheat, we used fastq-dump from the SRA-toolkit to download publicly available SRA files for subsequent analysis using the -split-files and -gzip parameters (Sherry et al., 2012). Wealso generated custom libraries from indTaRKD1 lines. Sequencing reads were pre-processed using fastp with default settings and the overrepresentation analysis parameter to remove reads with low quality score, irregular GC content, short length, and sequencing adapters present (Chen et al., 2018). Trimmed reads were then quality checked using FastQC v0.11.5 to ensure trimming had been successful (Andrews, 2010). Trimmed reads were then pseudoaligned using Kallisto against an indexed Triticum aestivum IWGSC RefSeq v1.1 cDNA genome under default parameters (Bray et al., 2015; IWGSC et al., 2018). Raw counts were imported into R and normalised before analysis using DESeq2 (Love et al. 2014; Team R, 2015). Replicate quality was verified using Principal Component Analysis (PCA) following a variance stabilised transformation of the samples and scatterplots of replicates were visualised using logarithmically transformed counts using DESeq2 functions and base R. Differentially expressed genes (DEGs) were calculated using DESeq2 with an FDR<0.05 and a IfcThreshold>0.5. Heatmaps were plotted using logarithmically transformed normalised counts with the pheatmap package using genes (rows) to scale (Kolde and Kolde, 2015). MA plots and volcano plots were made using DESeq2 functions and the EnhancedVolcano package (Blighe et al., 2020). Gene set enrichment analysis was performed using the gage package using genes that fit the DESeq2 dispersion plot with a set size of 800, rank test set to true with a non-paired comparison (Luo et al., 2009). GO terms enrichment analysis was calculated for all DEG list using gProfiler with a Benjimini-Hochberg correction set at a threshold of 0.05. GO term enrichment was visualised using a custom script with ggplot2 (Wickham 2011; Raudvere et al., 2019). The weighted gene coexpression network was constructed using transcript per million abundance (tpm) values over 0.5 from meristematic tissue samples taken from the JIC developmental timecourse. WGCNA and GGM were used to calculate weighted edge values for the 53,000 genes that passed the filter (Langfelder and Horvath, 2008; Schaefer et al., 2015). Average values from both functions were taken and normalised against randomly generated edge values for the genes used to create the final coexpression table. Edge values that had at least one of the indTaRKD1-7D RNAseq DEGs (set at IfcThreshold=0, p.adj=0.05) and had a value over 0.7 were filtered from the final coexpression table due to a high number coexpressed nodes below this value. The network was then visualised and annotated using Cytoscape 3.7.2 under an edge-weighted spring directed layout (Shannon et al., 2003). Nodes were annotated with a black border of 2 to indicate a DAPseq binding peak within 10 kb of the upstream promoter, coloured according to the log 2fold change value, and the size was increased if the node was a member of a transcription factor family. This coexpression was then uploaded to the nDEX database under the name ‘indTaRKD1-7D-logfoldchange’. Clusters were automatically curated using the AutoAnnotate cytoscape app and GO terms enrichment analysis was calculated for all clusters list using gProfiler with a Benjimini-Hochberg correction set at a threshold of 0.05 (Kucera et al., 2016; Raudvere et al., 2019). Clusters with significant GO terms were coloured for a separate network and the most significant non-redundant term was used to name this cluster. Revigo was used with default parameters to remove redundant GO-terms and plotted using an R script provided through the site. This coexpression network was then uploaded to the nDEX database under the name ‘indTaRKD1-7D-GO term’.

### Scanning Electron Microscopy (SEM)

For scanning electron microscopy (SEM) analysis, 2-3 mm immature inflorescences were isolated, those were submerged in fixative solution (2% paraformaldehyde, 2.5% glutaraldehyde, 25 mM phosphate buffer (pH 7)), and incubated at 4° C. for 16 hours. After fixation, samples were washed with 25 mM phosphate buffer three times for 10 minutes each with gentle agitation, were dehydrated via a graded ethanol series, and chemically dried using HMDS (Sigma, UK). Pre-treatment and SEM imaging were performed in Warwick Electron Microscopy Suite was used to perform sample coating with gold, and SEM imaging was performed using Jeol 6100 EDAX.

Spike x-Ray Computerised Tomography Scan

10 spikes from three genotypes from the spike traits trial were visualised by Dr. Paul Wilson of the Warwick Manufacturing Group (WMG). The wheat spikes were scanned on a Tescan Unitom XL Micro CT scanner. They were scanned at 40 kV and 1121 uA with an exposure of 70 ms over 1583 with 4 averages using Acquila (Tescan). The resulting radiographs were then reconstructed 3D voxelised volume using Acquila Reconstructor (Tescan), with a resulting voxel resolution of 50 um.

### Genome Wide Association Study

For the genome-wide association study we used shotgun sequencing data generated for 306 Ae. tauschii accessions (Gaurav et al., 2021). We first generated a k-mer presence/absence matrix. k-mers (k=51) were counted in trimmed raw data per accession using Jellyfish70 (version 2.2.6 or above). k-mers with a count of less than two in an accession were discarded. k-mer counts from all accessions were integrated to create a presence/absence matrix with one row per k-mer and one column per accession. The entries were reduced to 1 (presence) and 0 (absence). k-mers occurring in less than two accessions or in all but one accession was removed during the construction of the matrix. Association mapping plots were generated using Python. For a chromosome-level reference assembly, each integer on the x axis corresponds to a 10-kb genomic block starting from that position. For an anchored assembly, each integer on the x axis represents the scaffold that is anchored starting from that position. Dots on the plot represent the −log P values of the filtered k-mers within each block. Dot size is proportional to the number of k-mers with the specific −log P value.

Number of tillers was determined for each accession by growing 20 individuals for each accession in replicated plots.

### Example 1: Genome-Wide Association Study (GWAS) Identifies Major Loci Associated with Tiller Number in Aegilops tauschii (Ae. Tauschii)

### Introduction

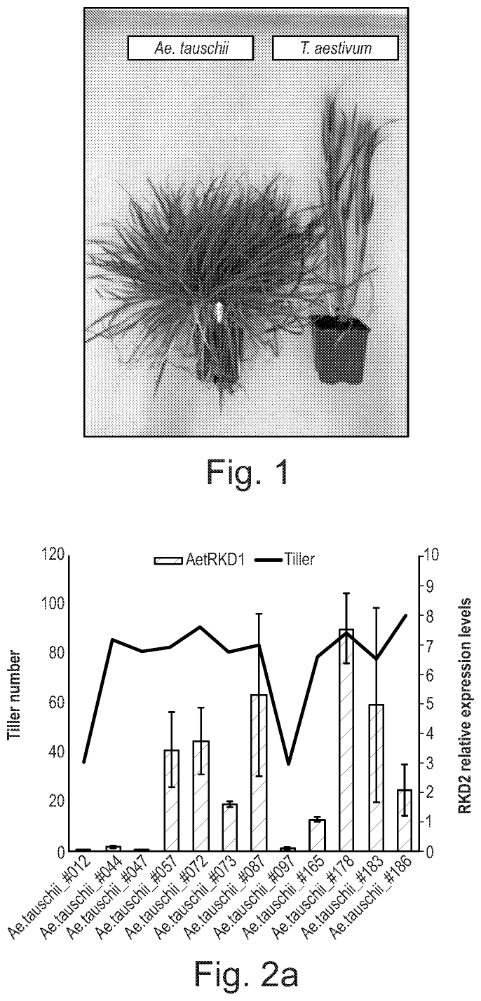

Plant architectures are different between allohexaploidy wheats such as Triticum aestivum and diploidy wheats such as Ae. tauschii. As shown in FIG. 1, Ae. tauschii is dwarf and bushy and whereas T. aestivum is upright and less tiller. Resultantly, a GWAS association study was performed to identify peaks involved with tiller number in Ae. tauschii.

### Methods

Using 200 Ae. tauschii accessions, a GWAS K-mer analysis was performed.

### Results

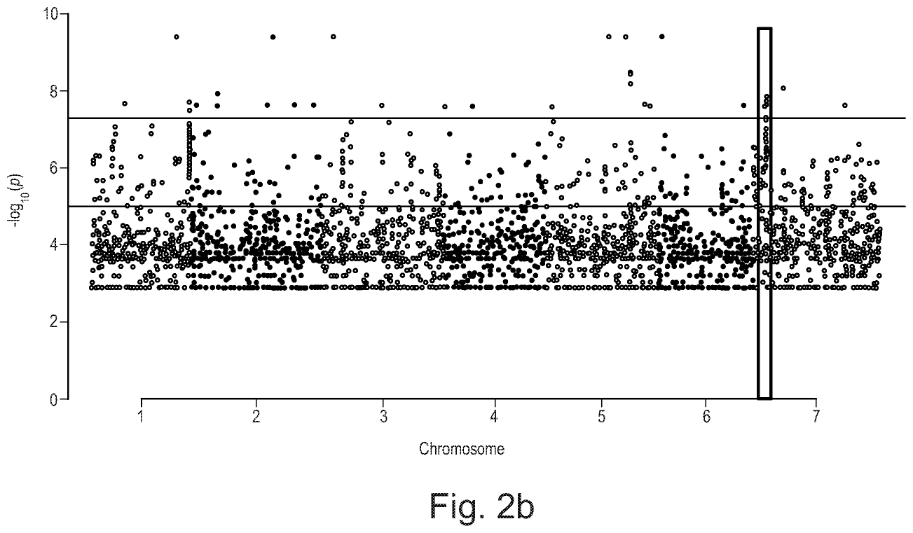

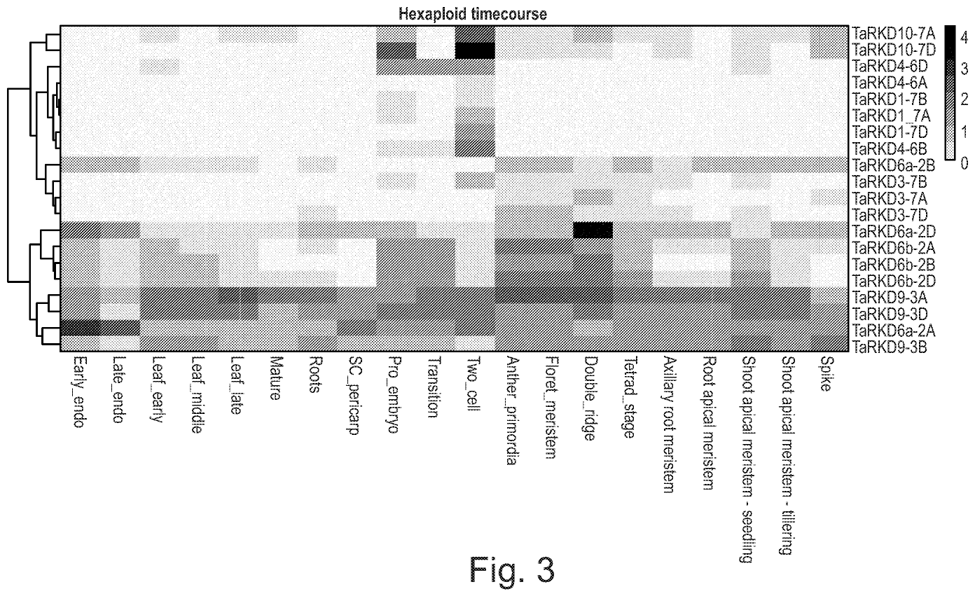

Tiller number in various Ae. tauschii accessions are shown in FIG. 2a. In the GWAS analysis shown in FIG. 2b. one high confidence locus was identified on chromosome 7D. At this locus, transcription factors including zygotic transcription factors (ZTFs) were identified and named wheat tiller factors (WTFs).

The factors identified are involved in cell reprogramming and meristem initiation.

### Example 2: TaRKD Transcription Factors

### Introduction

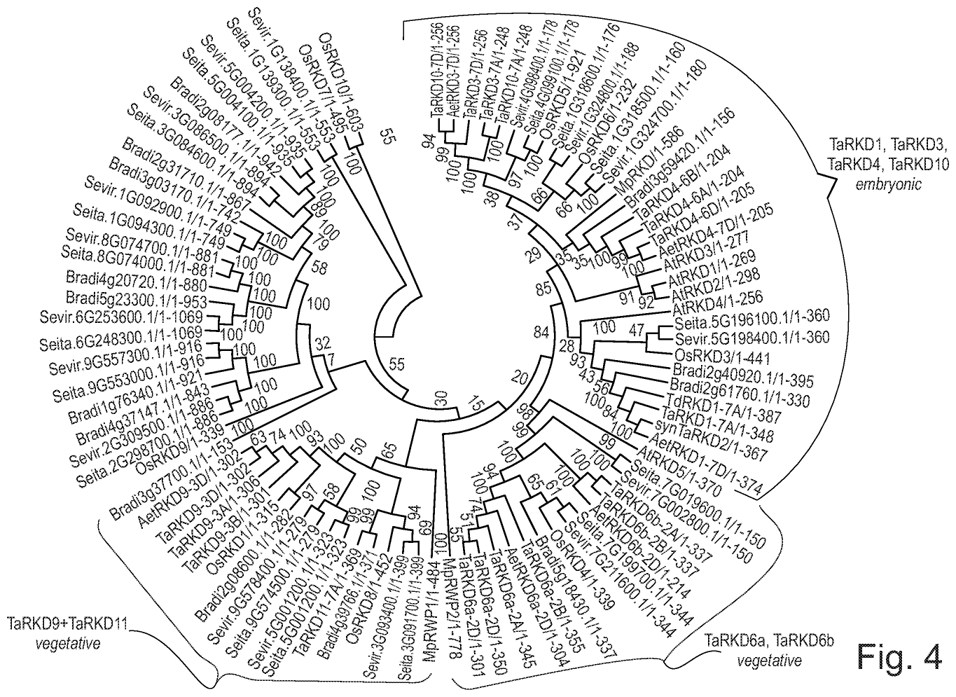

TaRKDs are a class of transcription factors, with homologues expressed in other plant species. TaRKDs are divided to two subclasses, reproductive and vegetative (see FIG. 4). TaRKD1 homologues are specifically expressed in embryonic cells and meristem tissues as shown in FIG. 3. A comparison of RKD1 expression in allohexaploidy and diploidy wheats was performed.

### Results

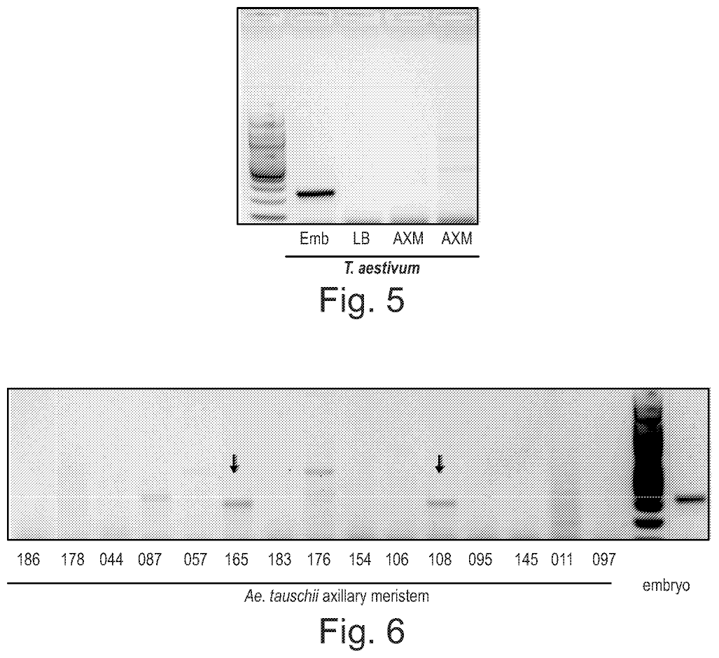

TaRKD1 is strongly expressed in immature embryos (Emb), but not expressed in leaf blades (LB) and axillary meristem (AXM) of T. aestivum as shown in FIG. 5. In Ae. tauschii, aeRKD1 is highly accumulated in the immature embryo as well (data not shown). Interestingly, AeRKD1 transcripts are also expressed in axillary meristems of some Ae. tauschii accessions. Ae. tauschii accessions have a greater number of tillers than the other accessions (FIG. 6).

### Example 3: Ectopic Expression of TaRKD1 in Hexaploidy Wheat

### Introduction

The inventors hypothesised that TaRKD1 control tiller formation in hexaploidy wheat. Therefore, a TaRKD1-OX transgenic lines were generated (#1.4_5, #1.4_2, #2.28_5 and #2.28_11.) to investigate the effect of ectopic expression of TaRKD1-OX transgenic lines were generated (#2.28_5B, #2.28_2B, #1.4_5D and #1.4_11A).

### Results

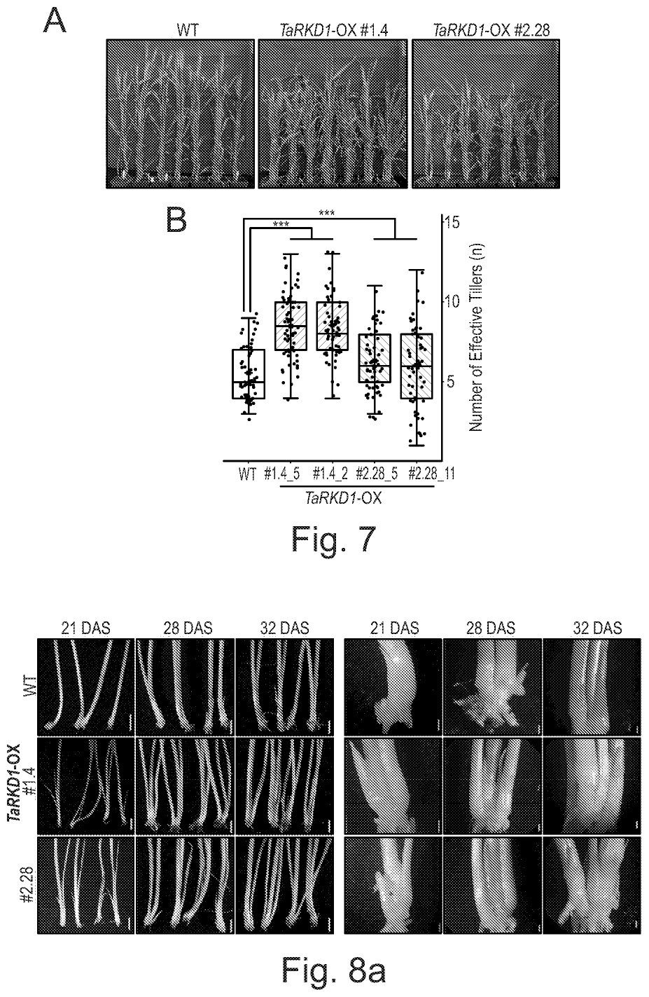

Hexaploidy wheat TaRKD1-OX lines with ectopic overexpression of TaRKD1 had a significantly increased number of effective tillers as compared to wild type (see FIGS. 7A and B).

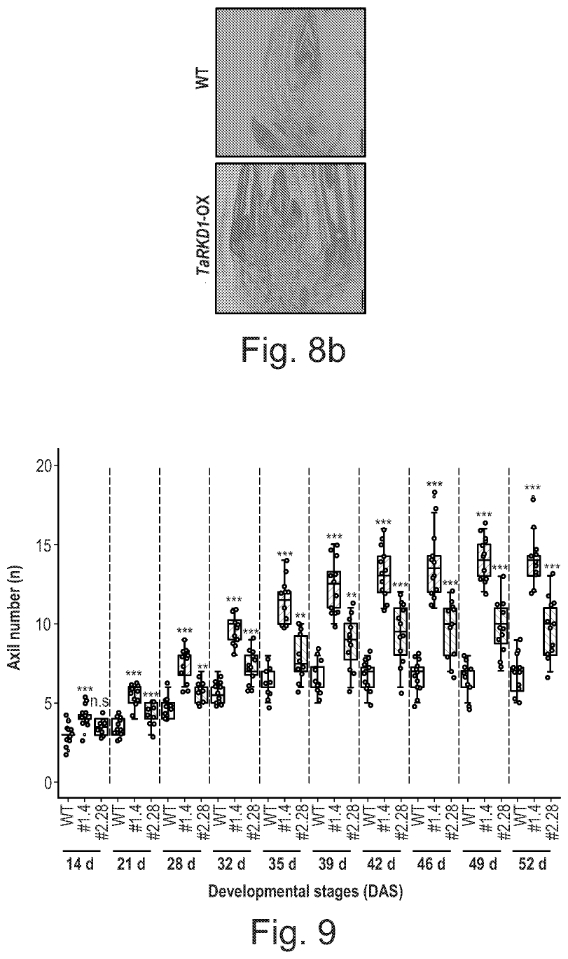

To further investigate the phenotype, axil numbers were monitored according to developmental stages. As early as 21 days after sowing (21 DAS) a difference in the number of axils between wild type and the transgenic lines (#2.28 and #1.4) was observed. At day 28 and day 32 after sowing, the number of axils is greater in the transgenic lines compared to wild type (FIG. 8a). The cell number in the axillary meristem was also increased in the TaRKD1-OX lines (FIG. 8b).

To numerically compare the number of axils between wild type and transgenic lines, the axils of several plants were counted. At day 14, a difference in the axil number was observed between WT and one of the TaRKD1-OX lines (FIG. 9). The axil number was significantly higher in the TaRKD1 expressing lines over the time course of the developmental stages from day 21 to day 52 (FIG. 9). Line #1.4 showed a more significant increase in axil number.

These findings demonstrate that TaRKD1 overexpression increases axil number through meristem initiation, and this results in an increase in tillers.

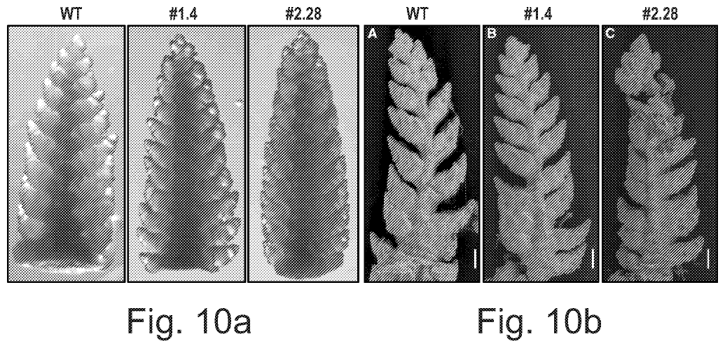

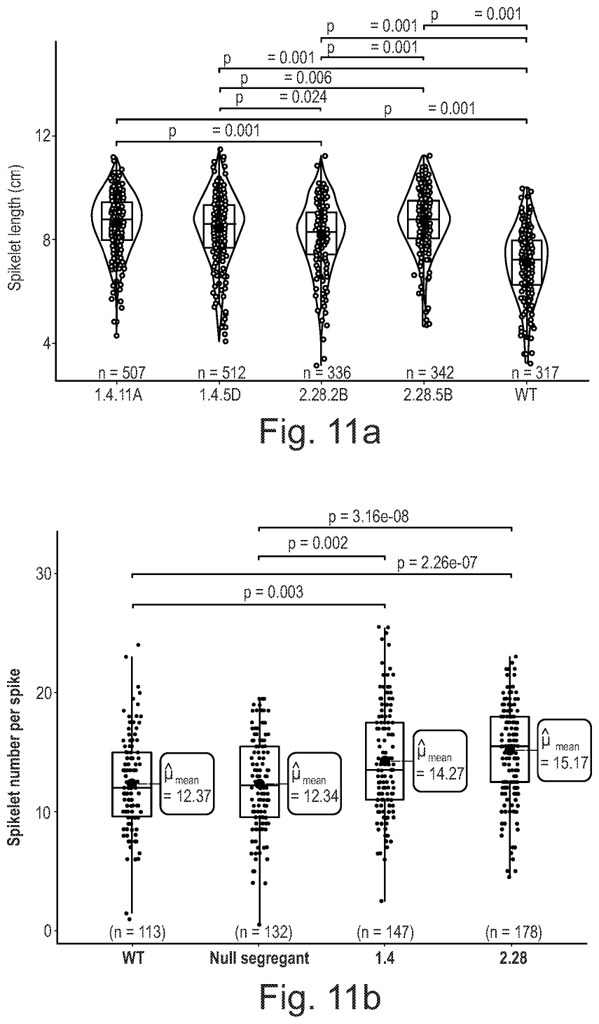

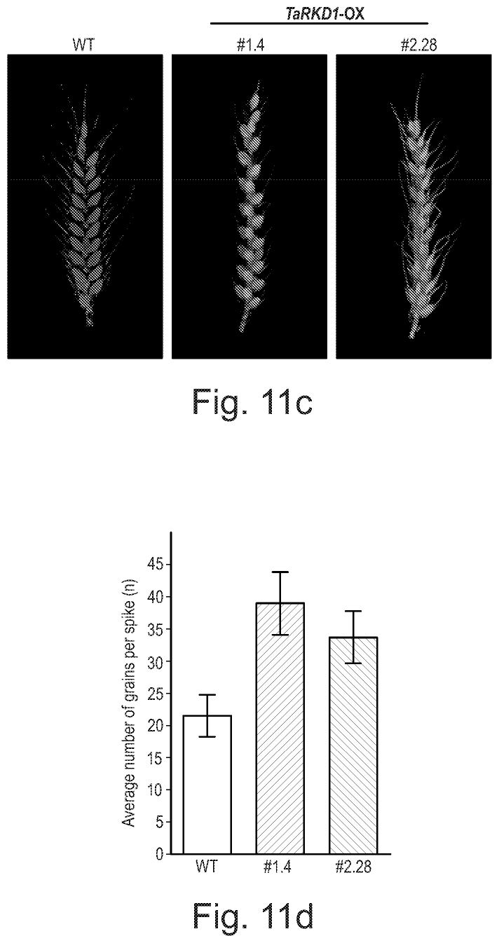

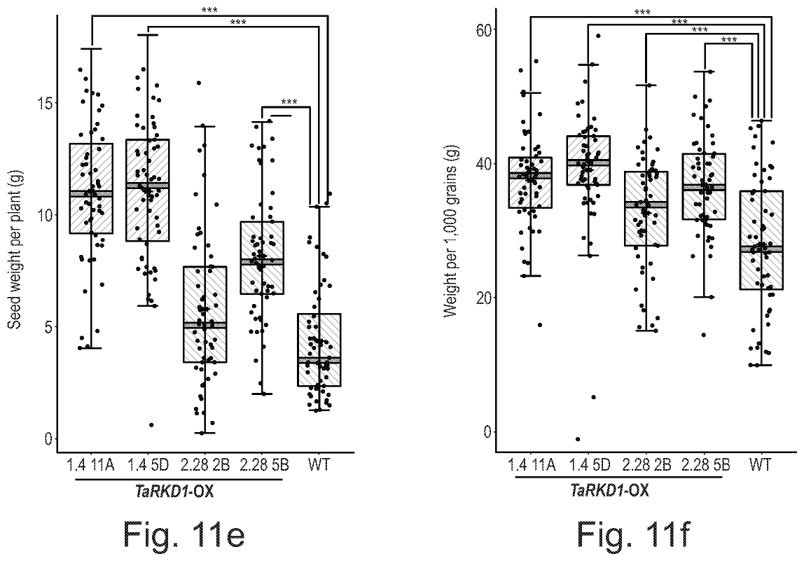

As meristem specification can determine other tissues, it was expected that other tissues might be changed in the transgenics. Overexpression of TaRKD1 increased spikelet number in mature spikes (FIG. 10a). The spike number was counted in each line and the spikelet number per spike was found to be significantly higher in TaRKD1 overexpressing lines. First, immature inflorescences were investigated, overexpression lines had more spikelet than WT. WT had usually 17 to 19 spikelet meristems, but transgenics had 21 to 23 meristems. Similar differences were also observed in SEM analysis (FIG. 10b). Spikelet length was also found to be significantly increased in TaRKD1-OX transgenic lines compared to wild type (FIG. 11a).

In mature spikes, spikelet number was also increased in overexpression lines. To allow for a quantitative comparison, spike number was counted in each line and was found to be significantly higher in the transgenics as shown in FIG. 11b.

As the number of spikelets and the number of tillers were increased upon ectopic expression of TaRKD1, the inventors hypothesized that the grain yield would also be increased.

The average number of grains per spike was found to be increased in the transgenic lines compared to wild type (FIG. 11c and FIG. 11d). The total seed weight per plant was also measured and found to be significantly increased in taRKD1-OX lines, most strikingly with the 1.4 line (FIG. 11e). Weight per 1,000 grains was also measured, also showing a significant increase in TaRKD1 overexpressing lines as compared to wild type (FIG. 11f). These results demonstrate that overexpression of TaRKD1 results in increased grain yield.

These finding show ectopic expression of TaRKD1 was able to induce initiation of several meristematic tissues, such as axillary meristem, inflorescence meristem, and spike meristem. It is expected that this factor plays a crucial role in meristem development or cell reprogramming.

### Example 4: Investigating the Molecular Function of TaRKD1 in Meristem Development

### Introduction

To investigate molecular function of TaRKD1 in meristem development, the inventors performed RNA-seq analysis in immature embryo tissues.

### Results

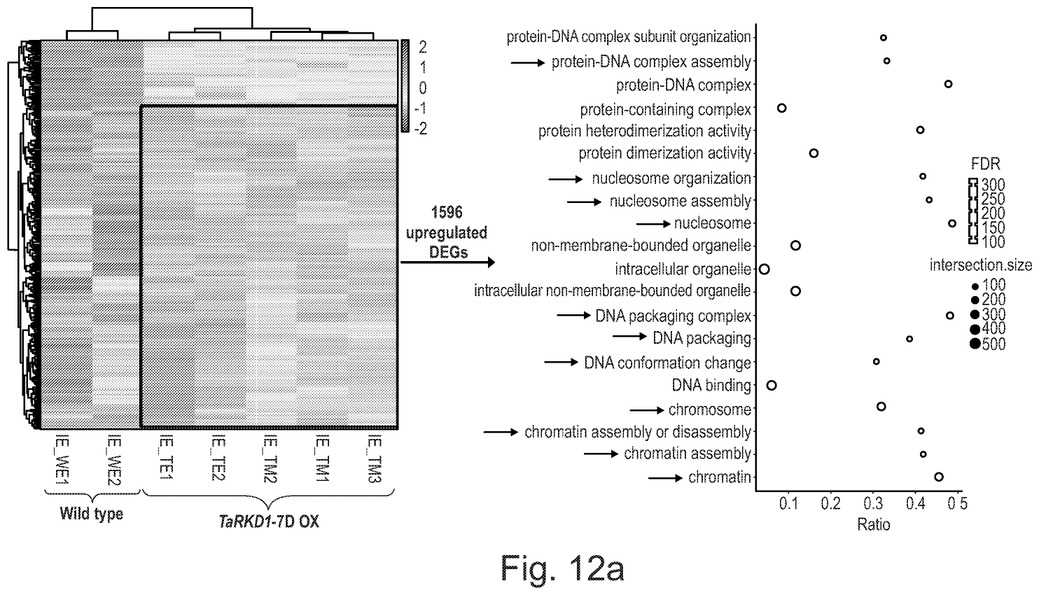

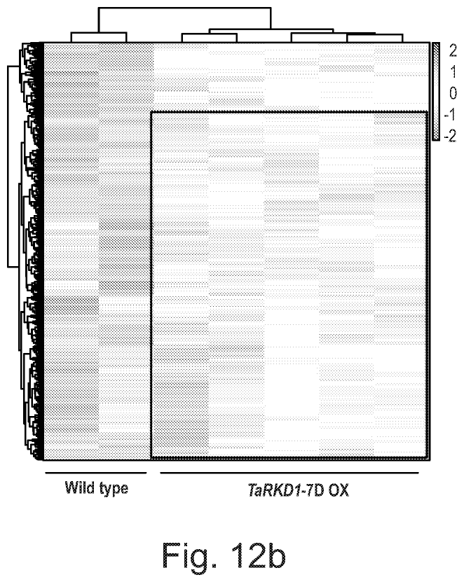

About 1,600 genes were found to be upregulated. Among this gene list were chromatin-related factors, such as factors regulating chromatin accessibility, chromatin assembly, DNA packaging, and protein-DNA complex assembly. This experiment demonstrated that TaRKD1 might be involved in chromatin accessibility and transcription initiation (FIGS. 12a and b).

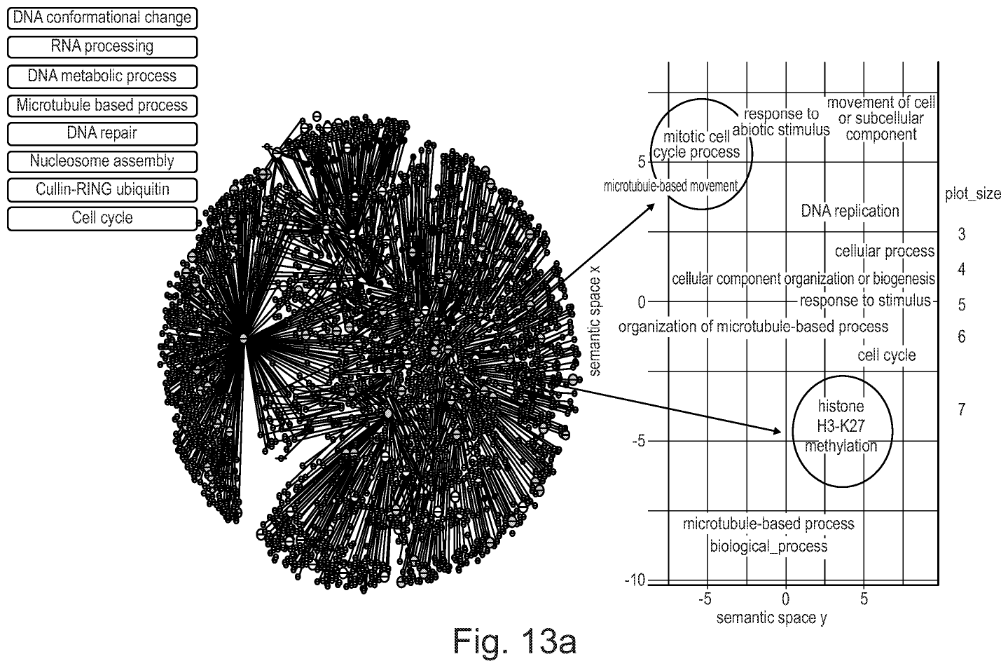

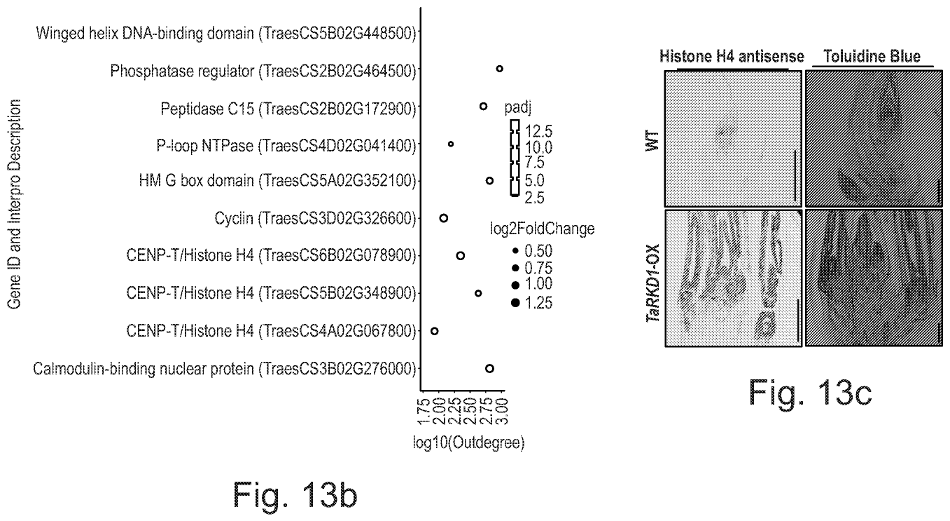

To further investigate the regulation, co-expression analysis for upregulated differentially expressed genes (DEGs) was performed and networks were constructed. Histone modification factors and cell cycling factors were enriched and DNA methylation factors and transcription factors were also abundant. Especially, cell cycle and histone H3K27me3 methylation are connected well in co-expression network (FIG. 13a).

Among top outdegree nodes in co-expression network, several Histone H4 genes were enriched as well (FIG. 13b). To verify those, RNA in situ hybridization was performed. Histone H4 signal was increased, and the signal was more spread in the transgenics (see FIG. 13c). Histone H4 genes in top outdegree nodes in co-expression network is upregulated in axillary meristem

Histone H4 also associates with cell division, and can be used as a cell division marker. H4 is highly expressed in cell divided tissues, such as meristems and embryos. These results demonstrated that TaRKD1 induces cell reprogramming via controlling chromatin accessibilities and initiates meristems through activating cell cycles.

### Example 5: Enrichment of TaRKD1 Binding in Promoter Regions with ChIPseq Showing More than DAPseq

### Introduction

As TaRKD1 is associated with histone modification markers, DAPseq and ChIPseq was performed to determine the regulatory mechanisms of TaRKD1 in meristem initiations using TaRKD1 antibody.

### Results

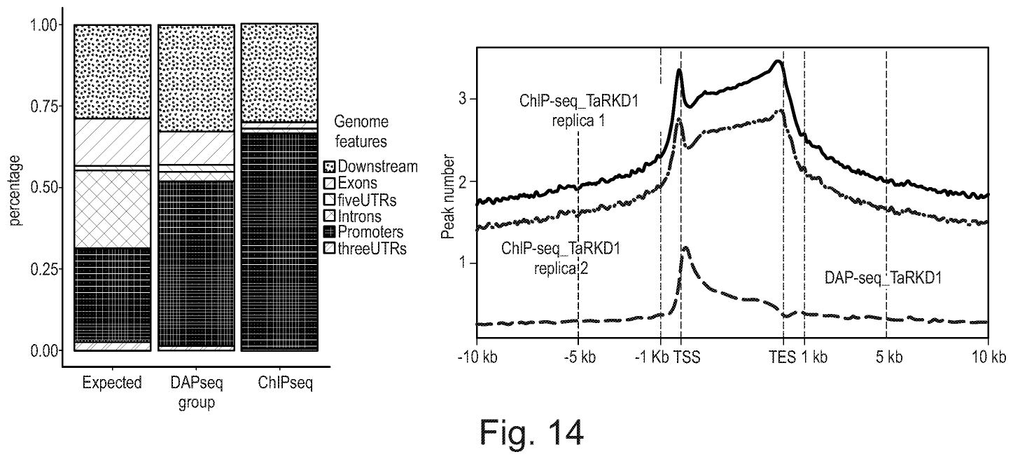

Enrichment of TaRKD1 binding in promoter regions was observed (FIG. 14).

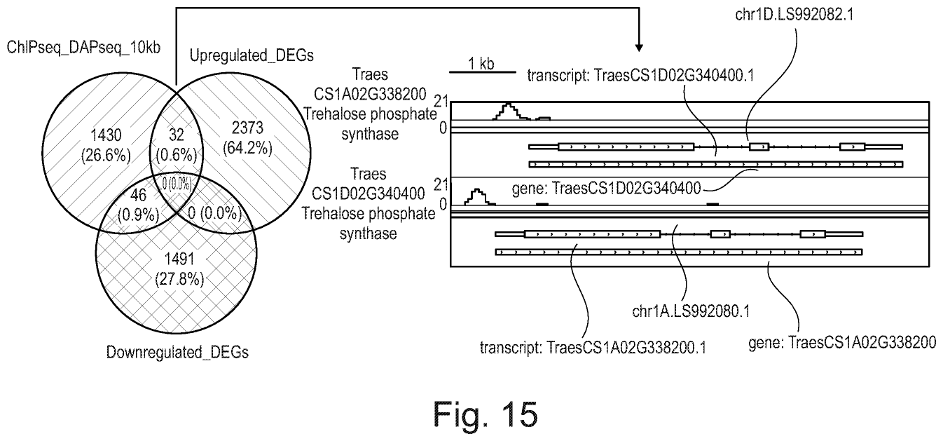

Although ChIPseq data has rarely overlapped with upregulated DEGs of RNAseq (FIG. 15), it was observed that TaRKD1 bound promoters of Trehalose phosphate synthase (FIG. 15), which is a meristematic determinacy factor.

Upregulated DEGs with ChIPseq and DAPseq peaks are trehalose-6-phosphate (T-6-P) synthases. RAMOSA3 encodes a T-6-P phosphatase in maze and decreases the abundance of T-6-P. ra3 mutant shows decreased meristematic determinacy, and upregulating T-6-P synthases should decrease meristematic determinacy, causing increased meristem formation and maintenance. T-6-P has also been shown to induce axillary meristem formation in peas suggesting it is evolutionarily conserved.

### Example 6: The Functions of RKD Transcription Factors in Meristem Formation are Conserved in Rice

### Introduction

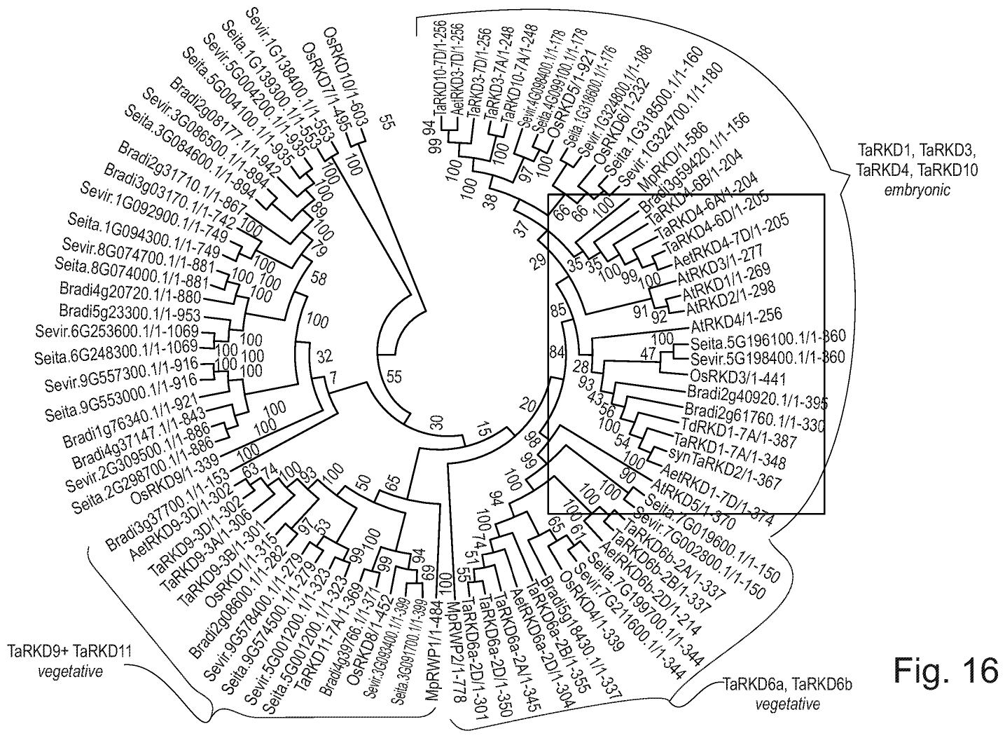

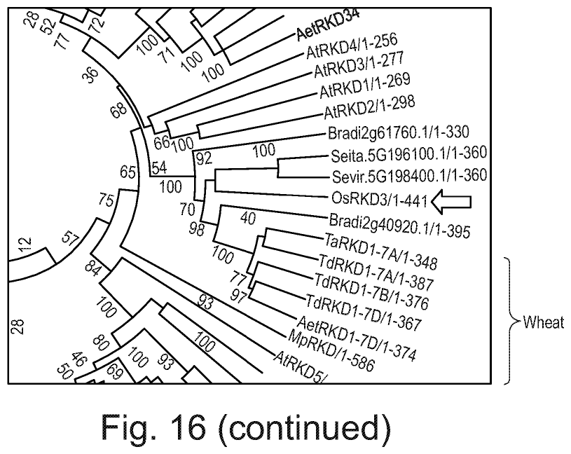

To investigate the functional conservation of RKD transcription factors, a phylogeny tree was studied and OsRKD3, a reproductive TaRKD, was identified in rice. This rice OsRKD3 is grouped with wheat reproductive RKD factors (FIG. 16). Thus, it was expected that OsRKD3 might have similar functions in meristem determinacy to wheat TaRKD1.

### Results

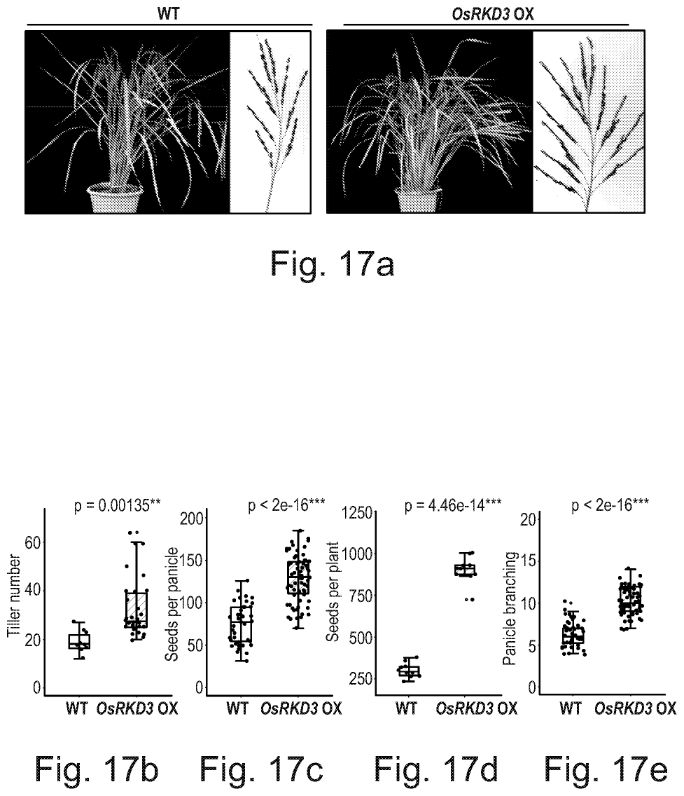

OsRKD3 overexpression transgenic rice plants were constructed and the phenotypes observed. As shown in FIG. 17a, tiller number increased in the transgenics (OsRKD3 OX) as compared to wild type. This was verified numerically, showing significant increases in the transgenic plants (FIG. 17b). The panicle branch number also increased (FIG. 17e), and this resulted in increase of seeds per panicle and seeds per plant (FIG. 17c &d). Resultantly, the grain yield also increased.

Overall, OsRKD3 overexpression increases grain yield via changes of tiller number and panicle branches, this is the same result as shown in the examples of overexpression of RKD factors in wheat above.

### Example 7: Rice Genes Encoding for Proteins with RWP-RK Domains

As OsRKD3 overexpression is associated with increased grain yield via increases in panicle branches and tiller number, the inventors further identified rice genes that encode for proteins with RWP-RK domains.

Table 1 shows a list of rice genes that encode proteins with RWP-RK domains. NLP family transcription factors have a RWP-RK domain and are preferentially expressed in the vegetative tissues of rice plants. OsRKD03 (OsRKD3), OsRKD04 (OsRKD4), OsRKD05 (OsRKD5) and OsRKD06 (OsRKD6) are preferentially expressed in reproductive tissues.

TABLE 1

| Gene names | Locus_id_MSU | Reference |
|---|---|---|
| OsRKD01 | LOC_Os01g14420 | Schauser et al. 2005 |
| OsRKD03 | LOC_Os01g37100 | Schauser et al. 2005 |
| OsRKD04 | LOC_Os04g47640 | Schauser et al. 2005 |
| OsRKD05 | LOC_Os06g12360 | Chardin et al. 2014 JXB |
| OsRKD06 | LOC_Os02g51090 | Chardin et al. 2014 JXB |
| OsRKD07 | LOC_Os08g19820 | Chardin et al. 2014 JXB |
| OsRKD08 | LOC_Os12g12970 | Chardin et al. 2014 JXB |
| OsRKD09 | LOC_Os09g27190 | Chardin et al. 2014 JXB |
| OsRKD10 | LOC_Os02g20530 | Chardin et al. 2014 JXB |
| OsNLP01 | LOC_Os03g03900 | Schauser et al. 2005 |
| OsNLP02 | LOC_Os04g41850 | Schauser et al. 2005 |
| OsNLP03 | LOC_Os01g13540 | Schauser et al. 2005 |
| OsNLP04 | LOC_Os09g37710 | Chardin et al. 2014 JXB |
| OsNLP05 | LOC_Os11g16290 | Chardin et al. 2014 JXB |
| OsNLP06 | LOC_Os02g04340 | Chardin et al. 2014 JXB |

### Example: 8 the Functions of RKD Transcription Factors in Meristem Formation are Conserved in Arabidopsis

AtRKD4, an Arabidopsis homologue of TaRKD1, overexpression lines were generated to investigate if the function of RKD transcription factors in meristem formation are well conserved in further plant species.

### Results

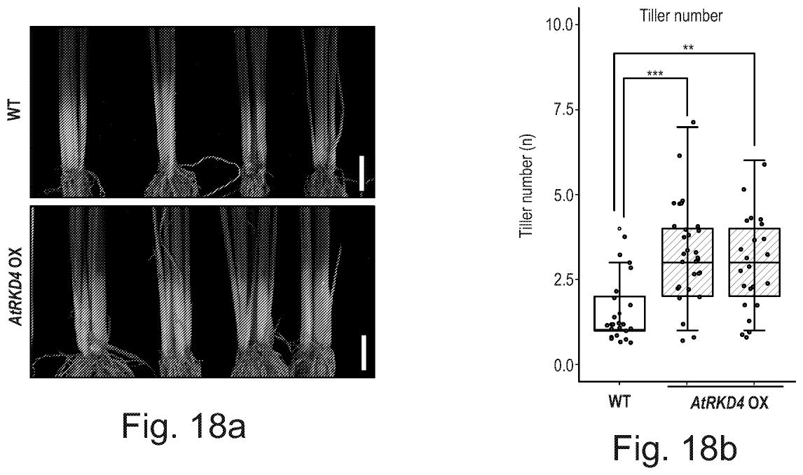

AtRKD4-overexpression resulted in a phenotype similar to that observed OsRKD3 and TaRKD1 overexpressed lines. Ectopic overexpression of AtRKD4 (AT5G53040; SEQ ID NO: 39) increased the tiller number and the axil number in Arabidopsis plants (FIG. 18 a&b).

These results demonstrated that functions of RKD transcription factors in meristem formation, and therefore in controlling yield-related traits, are well conserved in several plant species, thus demonstrating this factor can be used as a tool to improve yield in crop species that are both monocot and dicot.

### Sequences

### Reproductive Triticum Aestivum Tarkds

TaRKD1-7A
(SEQ ID NO: 1)
MEMQQYFGGCGDGDADWFHQLALLPPLPVSSSLPPLPMRPEEQMGCLQ
MIPPQAVADDEYSSYATNNVDVLPPFPAGLDDPTAGLDDALLMESFRD
IDLEEFADAVGPKIKTEPLDDAMVPADHDFAAQVQQARPVVIMNQQQL
NAPHGVRLLNDPDDDDSAVVAGGYEAAAVGCAEQKQVRPAPRRVRKSS
GGSRPAAGGKSLDHIGFEELRTYFYMPITKAAREMNVGLTVLKKRCRE
LGVARWPHRKMKSLRSLILNIQDMGKGATSPAAVQGELEALERYCAIM
EENPAIELTEQTKKLRQACFKENYKRRRAAASVNLLEHCYNDLGSHEQ
QMPLPQMGFFGF
Conserved RKD motif is shown in bold and
underlined
Conserved RKD motif:
(SEQ ID NO: 2)
RWPHRK
Conserved RKD motif and flanking regions:
(SEQ ID NO: 3)
KSSGGSRPAAGGKSLDHIGFEELRTYFYMPITKAAREMNVGLTVLKKR
CRELGVARWPHRKMKSLRSLILNIQDMGKGATSPAAVQG
TaRKD1-7B
(SEQ ID NO: 4)
MEMQQYYGGGGGCDGDGDWFHQLAALPPLPMSSSLPPPLLMSEGSCLP
MAAAAALPLGDCSSALMIRPEEQMGCLQMIRPPAVADDVYSSYGPNNV
DVLPPFPAGLDDPTAGLDDALLMESFSDIDLEEFADAVGHKIKTEPLD
DAMVPADHDFAAQAQQACPVVIMNQQQLNAPRNVRLLKDPDDNDSTVV
AGGYEAAAVGCAEQKQVRPAPRRVRKSSGGARPAAGGKSLDHIGFEEL
RTYFYMPITKAAREMNVGLTVLKKRCRELGVARWPHRKMKSLRSLILN
IQEMGKGATSPAAVQGELEALERYCAIMEENPAIELTEQTKKLRQACF
KENYKRRRATASVNLLEHCYNDLGSHEQQMPLPQMGFFGF
Conserved RKD motif and flanking regions:
(SEQ ID NO: 5)
KSSGGARPAAGGKSLDHIGFEELRTYFYMPITKAAREMNVGLTVLKKR
CRELGVARWPHRKMKSLRSLILNIQEMGKGATSPAAVQG
TaRKD1-7D
(SEQ ID NO: 6)
MEMQQQYFGGDGDADWFHQLALLPPLPISSSLPPLPMSEGSCLPMAAA
AAAALPLGDCSSALMIRPEEQMSCLPMNPSPAVVDDVYSSYAPNNVDV
LPPFPAGLDDALLMESFSDIDLEEFADAFGHKIKTEPLDDAMVPADHD
FAAQAQQACPVVIMNQQQLNAPRDVRLLIDPDDDDSTVVAGGYEAAAV
GCAEQKQVRPAPRRVRKSSGGARPAAGGKSLDHIGFEELRTYFYMPIT
KAAREMNVGLTVLKKRCRELGVARWPHRKMKSLRSLILNIQEMGKGAT
SPAAVQGELEALERYCAIMEENPAIELTEQTKKLRQACFKENYKRRRA
AASVNLLDHCYNDLASHEQQMPLPQMGFFGF
Conserved RKD motif and flanking regions:
(SEQ ID NO: 5)
KSSGGARPAAGGKSLDHIGFEELRTYFYMPITKAAREMNVGLTVLKKR
CRELGVARWPHRKMKSLRSLILNIQEMGKGATSPAAVQG
TaRKD3-7A
(SEQ ID NO: 7)
MDSSGEHSFWPYGPSVWHDSYLLEEETLLCTLPFPSYGCQPLCSISPA
VAQSSSLQAPCLVPCDTDELDTSFDDDLLRCWEAIEQSDDSTEESGKG
LPLLCYGEVNGAASNAMRADRVRSERVLTFELVSQYFYMPITQAAREL
NVGLTVLKKRCRELRIPRWPHRKLKSLQTLINDVEVLQEAGKANDDGQ
LRAMVEMLEQERRLLEQRPYVELEEKTKRLRQACFKASYKKRRLLALE
PGEASKYY
TaRKD3-7D
(SEQ ID NO: 8)
MFQISLERMDSNGEHSFWPYGPSVWHDSYLLEDEALLCSLPFLSFGCQ
PLCSNSPAVAQSSSLQAPCLVPCDTGELDTSFDDDLLRCWEAIEQSDD
STEDSGKGLPLLCYGEESGAASNAMRADRVRSERVLTFELVSQYFYMP
ITQAARELNVGLTVLKKKCRELGIPRWPHRKLKNLQTLINDVEVLQEA
GKANDGEQLRAMVEMLEQERRLLEQRPYVELEEKTKRLRQACFKASYK
KRRLLVLEPGEASKYY
TaRKD4-6A
(SEQ ID NO: 9)
MYLLRQWSFEYLLEISLFYPTHRLCDTSTSSTTTATQSCPLQDQQEFE
ARLEVHIMSLLAALEGEHHGGEKELKGEVGRDAGEKKRDGPEEKPLLL
FQQVSRCFCMPIKQAAEELNVGLTLLKRQCRELGIPRWPHRKLKSLET
LIKNAQELGKPMIEVEMLQRKKKLIEERPGHIELDEETKVLRQACFKE
KFKRRRLMAMER
TaRKD4-6B
(SEQ ID NO: 10)
MYLLQQWSFEYLLEISLFYPTQHLCDTSTSSTTTTSQSYLLQDQQELE
ARLEVHIMSLLAALEGEHHGGEKVLKGEVGRDAGEKKRNGPEEKPLLS
FQQVSQCFCMPIKQAAEELNVGLTLLKRQCRELGIPRWPHRKLKSLET
LIKNAQELGKPMVEVEMLQRKKKLIEERPGHIELDEETKVLRQACFKE
KFKRRRLMAMER
TaRKD4-6D
(SEQ ID NO: 11)
MYLLRQWSFEYLLEISLFYPTQHLCDTSTSSTTTTTSQSCLLQDQQELE
ARLEVHIMSLLAALEGEHHGGEKVMKGEVGRDAGEKKRNGPEEKPLLSF
QQVSRCFCMPIKQAAEKLNVGLTLLKRQCRELGIPRWPHRKLKSLETLI
KNAQELGKPMIEVEMLRRKKKLIEERPGHIELDEVTKVLRQACFKEKFK
RRRLMAMEG
TaRKD10-7A
(SEQ ID NO: 12)
MDSSGEHSFWPYGPSVWHDSYLLEEETLLCTLPFPSYGCQPLCSISPAV
AQSSSLQAPCLVPCDTDELDTSFDDDLLRCWEAIEQSDDSTEESGKGLP
LLCYGEVNGAASNAMRADRVRSERVLTFELVSQYFYMPITQAARELNVG
LTVLKKRCRELRIPRWPHRKLKSLQTLINDVEVLQEAGKANDDGQLRAM
VEMLEQERRLLEQRPYVELEEKTKRLRQACFKASYKKRRLLALEPGEAS
KYY
TaRKD10-7D
(SEQ ID NO: 13)
MFQISLERMDSNGEHSFWPYGPSVWHDSYLLEDEALLCSLPFLSFGCQP
LCSNSPAVAQSSSLQAPCLVPCDTGELDTSFDDDLLRCWEAIEQSDDST
EDSGKGLPLLCYGEESGAASNAMRADRVRSERVLTFELVSQYFYMPITQ
AARELNVGLTVLKKKCRELGIPRWPHRKLKNLQTLINDVEVLQEAGKAN
DGEQLRAMVEMLEQERRLLEQRPYVELEEKTKRLRQACFKASYKKRRLL
VLEPGEASKYY

Vegetative Triticum aestivum TaRKDs

TaRKD6a-2A
(SEQ ID NO: 14)
MDAAAAAVTTLSALAVFASTLHQGAVRSVHGYRVVAMGGRCAWDRWVER
EFAFSPTSCREVPLPAAAPRILPVEWRGRPAYREGQVLGAWRCVLAFDS
IAAVPLPPTPPPMLCPFRNPGLVCVPSLYNDLYMVYQFQKFQQAPELVK
RDSDEKSTRLEADDKTSDVEVKEESGSDSDEDPQSGEELPAHVERRRRA
KKQHIASITLADIAPYFHLPIREASRALKIGVSILKQKCRQYGIPRWPH
RKIKSLDSLISDLEFVMDDTDGDVVQKETRRQREKEREKEKEKEKEKQE
AIQALAKRKRLLESEKEIIQQKPALDLMAETKLFREDVFKRRYRAKRLP
GQ
TaRKD6a-2B
(SEQ ID NO: 15)
MVEYGYKFSSKKIKQELRYTCISTFRFNFLAFCIASMDAAAAAAVTTLS
ALAVFASPLHQGAVRSVHGYRVVAMGGRCAWDRWVEREFAFSPTSCREV
PLPAAAPRILPVEWRGRPAYREGQVLGAWRCILAFDSIAAVPLPPMPPP
MLCPFRNPSLVFVPSLYNDLYMVFQFQKFQQAPELVKRDSDEKSTRLDA
EDKTSHAERRRRAKKQHIASITLVDIAPYFHLPIREASRALKIGVSILK
QKCRQYGIPRWPHRKIKSLDSLISDLEFVMDDTDGDVVQKETRRQREKE
KEEKEKEKQEAIRALAKRKRLLESEKEIIQQKPALDLMAETKLFREDVF
KRRYRAKRLPGR
TaRKD6a-2D v1
(SEQ ID NO: 16)
MDAAAAAAVTTLSALAVFASTLHQGAVRSVHGYRVVAMGGRCAWDRWVE
REFAFSPTSCREVPLPAAAPRILPVEWRGRPAYREGQVFGTWRCVLAFD
SIAAVPLPPTPPPMLCPFRNPGLVCVPSLYNDLYMVYQFQKFQQVPELV
KRDSDEKSTRLDAEDKTCDVEVKEESGSDSDEDPQSGEELPAHVARRRR
AKKQHIASITLADIAPYFHLPIREASRALKIGVSILKQKCRQYGIPRWP
HRKIKSLDSLIGDLEFVMDDTDGDVVQKETSRQREKEREKEKEKEKEKE
KEKQEAIQALAKRKRLLESEKEIIQQKPALDLMAETKLFREDVFKRRYR
AKRLPGH
TaRKD6a-2D v2
(SEQ ID NO: 17)
MGGRCAWDRWVEREFAFSPTSCREVPLPAAAPRILPVEWRGRPAYREGQ
VFGTWRCVLAFDSIAAVPLPPTPPPMLCPFRNPGLVCVPSLYNDLYMVY
QFQKFQQVPELVKRDSDEKSTRLDAEDKTCDVEVKEESGSDSDEDPQSG
EELPAHVARRRRAKKQHIASITLADIAPYFHLPIREASRALKIGVSILK
QKCRQYGIPRWPHRKIKSLDSLIGDLEKETSRQREKEREKEKEKEKEKE
KEKQEAIQALAKRKRLLESEKEIIQQKPALDLMAETKLFREDVFKRRYR
AKRLPGH
TaRKD6b-2A
(SEQ ID NO: 18)
MEAPAAAAAAASTLSALAVFVSTVDRGAVRSAHGYRVAGKGGGCGWERW
VEREFAFTPTLAREVPLPADALRLLPADWRGRPVYREGQIAGPWRCILA
FDFVAAVAPPPVPPPVLCPFRNPRLMCVPSLYNDLTKVFQFQKVEEKVP
GLVQCDSDEKLTTSDAKDRVSVKQEAGSDTDEPPQFGEDLLAPAEKQRR
AHREYIASITLEDITQYFHLPIREASRTLKIGLSILKKKCRQYGIPRWP
HRKLKSLDSLIHDLEFVIDDETERDGVKQEDHMQDEKENQDAIEALAKR
KRMLETEKATIQQKPTLDLMAETKQFRQYVFKRKYRAKTLVSE
TaRKD6b-2B
(SEQ ID NO: 19)
MEAPAAAAAAVSTLSALAVFVSTVDRGAVRSAHGYRVAGKGGGCGWERW
VEREFAFTPTSAREVPLAADAQRLLPADWRGRPVYREGQIAGPWRCILA
FDFVAAVAPPPVPPPVLCPLRNPRLMCVPSLYNDLTKVFQFQKVEEKVP
GLVQCDSDEKLTSSDAKDRVSVKQEAGSDTDERPQFGEDLLAPAEKQRR
AHRAYIASITLEDIAQYFHLPIREASRTLKIGLSILKKKCRQHGIPRWP
HRKLKSLDSLIHDLEFVIDDETERDGVKQEDHMQDEKENQDAIEALAKR
KRMLETEKATIQQKPTLDLMAETKQFRQYVFKRKYRAKTLVSE
TaRKD9-3A
(SEQ ID NO: 20)
MSAPPMAASAQELPPAQQQPAAEQAPSLALALVPQPQAQAQIKPTRVSL
SYEEISKLFSLPIAEAASILGVCTSVLKRICRTHGIVRWPYRKIVSGKG
DDAKNAEREKAMQLLELSKIAKQKAISSSGSLATSSSGAFQGVPKAQQG
SAKAGTAIGRQNAPSLSQFSQAKDIPTYMDDFKYGFPSSGLSTETMKWW
GTDSHTETAPAKDDNREGSESTNEASKGTDDELDWGADEPEADADGVAA
ATDPSAQLCSLRRKAAGDGRRLLNGDTGRVQQLCRLNKRQKIVLAQVFG
ASLPEQRRSKLA
TaRKD9-3B
(SEQ ID NO: 21)
MSAPPMAASAQELPPAEQAPSLALALVPQPQAQAQIKPTRVSLSYEEIS
KLFSLPIAEAASILGVCTSVLKRICRTHGIVRWPYRKIVSGKGDDAKNA
EREKAMQLLELSKIAKQKAISSSGSLATSSSGAFQGVPKAQQGSAKAGT
AIGRQNAPSLSQFSQAKDIPTYMDDFKYGFPSSGLSTETMKWWGTDSHT
ETAPAKDDNREGSESTNEASKGMTDDELDWGADEPEADADGVAAATDPS
AQLCSLRRKAAGDGRRLLNGDTGRVQQLCRLNKRQKIVLAQVFGASLPE
HWRSNLA
TaRKD9-3D
(SEQ ID NO: 22)
MAASAQELPPAQQQPAAEQPPSLALALVPQPQAQAQIKPTRVSLSYEEI
SKLFSLPIAEAASILGVCTSVLKRICRTHGIVRWPYRKIVSGKGDDAKN
AEREKAMQLLELSKIAKQKAISSSGSLATSSSGAFQGVPKAQQGSAKAG
SAIGRQNAPSLSQFSQAKDIPTYMDDFKYGFPSSGLSTETMKWWGTDSH
TETAPAKDDNREGSESTNEASKGMTDDELDWGADEPEADADGVAAATDP
SAQLCSLRRKAAGDGRRLLNGDTGRVQQLCRLNKRQKIVLAQVFGASLP
EQWRSKLA
TaRKD11-7A
(SEQ ID NO: 23)
MLMATGDGADGLGLDDDSLPLQDNFDDVLQLLDDFPDPDTAPIPLPAQM
HHADQPACSHSFSGDGLRNNSTGDGTPGVAQPNEFPVHSDPSTSSTAAG
VLDCSGCHVLREVMHSNGPELTKLSIHGAPGFFNHATLDRCTNSEGLAP
RIAPRSHIDFRDRDYEWVGHFLTDYALRQAAGNYAVVRDSLSLFLDVLC
TTMNRCVQGNDGDHDDEGTAARNGGHGQPTVDVFAAAVQPAMQRNTEPE
PAGPSPSPPSNTMELHVQPQPFQPVVAGRSVLALQRERTRKMQFHDIAP
YFHLPIVEAAEKLDICTTVLKGICRRVGVQRWPHRKVKKIDRQITKLMR
SGNGVWERNEIERLNAERKRIFYALE

Reproductive Aegilops tauschii AeRKDs

AetRKD1-7D
(SEQ ID NO: 24)
MEMQQQYFGGDGDADWFHQLALLPPLPISSSLPPLPMSEGSCLPMAAAA
AAALPLGDCSSALMIRPEEQMSCLPMNPSPAVVDDVYSSYAPNNVDVLP
PFPAGLDDPTAGLDDALLMESFSDIDLEEFADAVGHKIKTEPLDDAMVP
ADHDFAAQAQQACPVVIMNQQQLNAPRDVRLLIDPDDDDSTVVAGGYEA
AAVGCAEQKQVRPAPRRVRKSSGGARPAAGGKSLDHIGFEELRTYFYMP
ITKAAREMNVGLTVLKKRCRELGVARWPHRKMKSLRSLILNIQEMGKGA
TSPAAVQGELEALERYCAIMEENPAIELTEQTKKLRQACFKENYKRRRA
AASVNLLDHCYNDLASHEQQMPLPQMGFFGF
AetRKD3-7D
(SEQ ID NO: 25)
MFQISLERMDSNGEHSFWPYGPSVWHDSYLLEDEALLCSLPFLSFGCQP
LCSNSPAVAQSSSLQAPCLVPCDTGELDTSFDDDLLRCWEAIEQSDDST
EDSGKGLPLLCYGEESGAASNAMRADRVRSERVLTFELVSQYFYMPITQ
AARELNVGLTVLKKKCRELGIPRWPHRKLKNLQTLINDVEVLQEAGKAN
DGEQLRAMVEMLEQERRLLEQRPYVELEEKTKRLRQACFKASYKKRRLL
VLEPGEASKYY
AetRKD4-7D
(SEQ ID NO: 26)
MYLLRQWSFEYLLEISLFYPTQHLCDTSTSSTTTTTSQSCLLQDQQELE
ARLEVHIMSLLAALEGEHHGGEKVMKGEVGRDAGEKKRNGPEEKPLLSF
QQVSRCFCMPIKQAAEKLNVGLTLLKRQCRELGIPRWPHRKLKSLETLI
KNAQELGKPMIEVEMLRRKKKLIEERPGHIELDEVTKVLRQACFKEKFK
RRRLMAMEG

Vegetative Aegilops tauschii AeRKDs

AetRKD6a-2D
(SEQ ID NO: 27)
MGGRCAWDRWVEREFAFSPTSCREVPLPAAAPRILPVEWRGRPAYREGQ
VFGTWRCVLAFDSIAAVPLPPTPPPMLCPFRNPGLVCVPSLYNDLYMVY
QFQKFQQVPELVKRDSDEKSTRLDAEDKTCDVEVKEESGSDSDEDPQSG
EELPAHVARRRRAKKQHIASITLADIAPYFHLPIREASRALKIGVSILK
QKCRQYGIPRWPHRKIKSLDSLISDLEVNLKETSRQREKEREKEKEKEK
EKEKEKQEAIQALAKRKRLLESEKEIIQQKPALDLMAETKLFREDVFKR
RYRAKRLPGH
AetRKD6b-2D
(SEQ ID NO: 28)
MCVPSLYNDLTKVFQFQKVEEKVPGLVQCDSDEKLTTSDAKDRVSVKQE
AGSDTDEPPQFGEDLLAPAEKQRRAHREYIASITLNDIAQYFHLPIREA
SRTLKIGLSILKKKCRQYGIPRWPHRKLKSLDSLIHDLEYVIDDETERD
GVKQEDHMQDEKENQDAIEALAKRKRMLETEKATIQQKPTLDLMAETKQ
FRQYVFKRKYRAKTLVSE
AetRKD9-3D
(SEQ ID NO: 29)
MAASAQELPPAQQQPAAEQPPSLALALVPQPQAQAQIKPTRVSLSYEEI
SKLFSLPIAEAASILGVCTSVLKRICRTHGIVRWPYRKIVSGKGDDAKN
AEREKAMQLLELSKIAKQKAISSSGSLATSSSGAFQGVPKAQQGSAKAG
SAIGRQNAPSLSQFSQAKDIPTYMDDFKYGFPSSGLSTETMKWWGTDSH
TETAPAKDDNREGSESTNEASKGMTDDELDWGADEPEADADGVAAATDP
SAQLCFLRRKAAGDGRRLLNGDTGRVQQLCRLNKRQKIVLAQVFGASLP
EQWRSKLA

Rice (Oryza sativa) RKDs

OsRKD1
(SEQ ID NO: 30)
MCAAPMASTAQPQQQPQQQQEQQPVAAAAVPTPAPPASEAQPQKPTRVS
LSYEEISKLFSLPIAEAASILGVCTSVLKRICRSHGIVRWPYRKLVSGK
SGDDTKNAEREKAKGLLEISKVAKQKALSASGLSTVSPGAFQGVAKSQQ
GSSKAGQVSPPGKQNVLGGSAILSYGTQTKGIPTYMDDFKYGFPSSGLS
LQTMKWWGTDSHTETTPAKDDNGEAPESANEASKGMTDDELDWGADEAE
AEADADSAITTEPSAQLCSLRRKAVDDGRKLLTGKSCGGLELCRLNKRQ
KMALAQVFGASLPEQLRSKLG
OsRKD3
(SEQ ID NO: 31)
MEMHECCYYGGSIGGDWLNPLAAIPPPCSSSSSSWSSQLLLLSDHDDVL
LHSAGDHGGAVAGIGGACMTADLVVRDEEMEMAAGYLPVAASAAAAADV
DHYMYQQFQLEPDQFVSTLPAVAVAVAATAGGGSHDDELLRMPFTDIDL
DAFADARDVVVGVGEPKPSPQHTLDAAIALPAVGGGGAHHFGTQDDDVK
FDVTKQRNDAALAGDDSLSMVIVESYEMGMRRHAAEQEQEQKPKIITSA
ATTLTPLPLPPPPPPPPRVTRSRRDGSSAATAGGKTRLDHIGFEDLRRY
FYMPITKAAREMNVGLTVLKKRCRELGVARWPHRKMKSLKSLILNEMGS
KGMSAAAMRRELEALENCCALMERNPAVELTERTKKLRQACFKENYKRR
RAAAVDVLDLDHCFSFAAGHCHRHHHQQLALPPPPAAAADHRRRDFFGY
Conserved RKD motif and flanking regions
underlined.
OsRKD4
(SEQ ID NO: 32)
MDAAAAAVSTLSALAVFASTLDHGAVRSVHGYKVYGRGGRRRWERWVER
EFVLTPASCREVPAPVAPPRILPAEWRGRPAYREGQVVAAGAWRCILAF
DSAAAPPRTPPPVLSPFLNPRLMCVPSLYNDLEKVFRFQNVEKIPKLMQ
CDSEEKLSSWDARDKSSDEVHASESDSDDDLQSGEEEKPTVQKQRRANK
KHIASITLVDIAQYFHLPIREASRTLKIGVSILKRKCRQYNIPRWPHRK
IKSLDSLIQDLEYVIDDGDDHDDTGDDVQQEKHKQTAEEKQEAIMALTR
RKQMLETEKETIQQIPAMDLKVETKQFREDVFKRRYRAKKDLAND
Conserved RKD motif and flanking regions
underlined.
OsRKD5
(SEQ ID NO: 33)
MRHNRSLAALLRAGRYGAARRLFDALPARSVVTWNSLLAGLARRPDARA
AREFFDAMPVRDAVSWNTLLAAYSASPHPDHLAAARRLFDEMPQRDVVT
WNTLLGAYARRGLMDEARRLFDEMPQRNAASWNTMVTGFFAAGQVVKAL
DVFDAMPAKDSASLSTMVSGFTKNGMLHEAEELLTKRLSVTDMDKAVDA
YNTLIVAYGQAGRFSDAKRLFDMIPKGQYQHNMLKRKGFERNVVSWNSM
MICYIKAGDVCSARALFNEMPDKDLVSWNTMISGYTQASDMKESEKLFW
EMPDPDTVSWNLIIQGFMQKGEAEHARGFFDRMPERGTISWNTMISGYE
KNGNYISSVKLFSKMLEVGEIPDRHTFSSVLAACASIPMLGLGAQIHQL
VEKSFVPDTAISNALITMYSRCGALNDAEAIFKQMHTKKDLVSWNALIG
CYEHHGRATKALQLFKEMRRAKVMPTHITFVSLLSACVNAGLVSEGRMV
FDTMVHEYGIVARIEHYAALVNLIGRHGQLDDALEVINSMPMAPDRSVW
GAFLGACTAKKNEPLAQMAAKELSTINPDSSAPYVLIHNLHAHEGKWGS
AAVVREEMERQGIYKQPGYSWIDLEGKMHVFISGDTWHPNAQEIFSVLE
DWQWHNPMSLEMNQLTQVKLIAKRNNQIMDKQATATKMHKATNIRHIYS
KFKVDEQQNTSWWPYCTSLWPDSYLLEEEALFSSLSFPSFHPQPVYSTV
MQSNVLQDELGVIFEDDVLKYWDEMEQSENKVEKSEKGLPLLYYGDENG
AASKIMRDDVRSEEKALTFELVSQYFYMPITQAARELNVGLTLLKKKCR
ELGIPRWPHRKMKSLQTLINNVQVLQEASKANNEEQLRMLVEMLQEERR
LLEQKPYVQLEEKTKRLRQACFKANYKKRRLLALEAGEP
Conserved RKD motif and flanking regions
underlined.
OsRKD6
(SEQ ID NO: 34)
MEKWRFYGGHQFQSYSFHEEENLFQDWSLDYLLLGEDEPFFTHHFSTSV
HSNFVQDELYTLFDGDILSIWGDMKEDAYHRSDKDGGEKEEKLDHEKAM
ELQLQRLPSGRQSGEKTLTFELVSQYFCLPIKQAAQELNVGLTLLKRRC
RVLGIPRWPHRKVKSLETIIKNVQELGMETGQDEDNTRNAVEMLQQTKK
LIEQSPDAKLDDWTKMLRQACFKENYKRRRLLAIEG
Conserved RKD motif and flanking regions
underlined.
OsRKD7
(SEQ ID NO: 35)
MAMSRATCSTRPRISRCSPSRRWFVPVAAAIESHPTPAIHVVEREWPAQ
VQISRFDPCSPPPPARMLPGGQQQQPHPAPGGLDLRSDEIHALKQRRRP
PPNPQSSSPPEGLLGAANPDRPDDDELDAILSSFRDEAAHGDSGGGVAR
VQVLPVEQDANLVPSLPLRHGQLDCSRCHLVRHVMHVAVLPYVKFSLKL
VWEEAFHRIRRMYVISVRSNVYSLSKWTQEWASEFIARNIDTMRNNTNG
QLLDSGYSNLVESVRTNVNVPHTAVEVNLLQTIMSAPSADHHQNAADQV
AAPAAQPFSAAPPVALPPKAAPRKARKDRDYASMLVAVEEFYVAATSRP
VPNSDVEILESSHVSQQQDGGRAIIYPSLQARRGKTKQEVPRRNAKDVL
EYLSLARKETEKEINTLSSFDGIYRNDGTLSYLMTEVRRLNRKIWRLQK
NAPSTLSSRLLASVKEIDDIKVEKGRLYAQFISALKKLCRKKMDDGGSA
PSANN
OsRKD8
(SEQ ID NO: 36)
MAGDGGNGAADGGGEHLELDFFPSPLLDNFDVLELLDLSVLGDVDDDGA
PPAPAPVQMSVDGAGGGRINKPGSSVDDLVDWSTAFGNNCSKIDGEYGG
ASTSSSAAPALAPPQEDYCSGCQVLREVVHSNGLEITKLCIHGGVASGE
FYHAILDVYRVSASAPAPALAHHSIINFRGRGYDWVKQYLTEYALRRAG
GGFAVVQDSLSAFHDALCTTMAPCSSHVGDDDAHRRASSSAAAAAEERT
NGNGDHGQLVVHNAAVLPMLESSRCLVAADQAATTNNNGSGDRRLVVLD
TTAIQPPASGCILHMAFPSKSYQSSRPTTLPSRYQECTCNPVAYEMDGV
RSSLAPICWPELLGYNVTNREKTKQLQLSDIAPYFELPIAKAAKKLDIC
ATALKGICRKHGVLRWPYRKVRSIDRQIATLRRSGNGDATRNEIETLIA
SRRRIVAGLDQ
OsRKD9
(SEQ ID NO: 37)
MATGGSSSSSNVPSGGAAGGGAGSGKLPMVPVGSIECLRKRCRQLRVL
IHVNDHRKAVVVLHAGEDGKPDHVLVQNVSPEGEASVQSTYRIDVSGE
TPESQAEMLNDWYTSFRMDTTGVLYDSDQNVIYGVPRGHPGGDVPRSL
AILPPAPKKNQHGKAPATESNSSLVEEPLLLVQTDQPAAIGKRKKFTF
PDQRKRVKTMTKKDLESYFHITQKSAAHIGLSIGTTALKNLCRANDLP
RWPYRQIASLDNKFNNNLKKQITGWNLGKAVQGVTKAFKLRKEKEEFY
QKIMSSMPEQLQSIDEIVNSLPEADDDIDIEDDEDNDDVIEDNDDDNS
DEN
OsRKD10
(SEQ ID NO: 38)
MAGEHRFYDSNGDVSRYMLDDMEFLPLFGEAEAATTLPLPVLPDVEPL
PTAPAPAATAHVEPAPAADFGLGNPVLADLGFDVDLDFFPELNFQSPP
PPPAMNAGGYTHQVQASPPVMHHQQQQQQPLAPLPAAHGFAGQPAPAT
TMAPSGGDDGLFLAAPSRDAPCSPVMFNFMDFNVDMGDVDMDDVLMWA
DQDTHGAAAGGDTAPPVVVDEYADFVPFQAGDLDCSNCHLVREMMHAN
ASRTIYFLVHATGVGSFQHAIVDRRYTATGAEGLHFPGRQLLYFDLTN
HTIESASDFIASNVEKLKNDTTGHHFLDTGYNFSGAVRTDMANSHTAM
EMNMLHTIVSAPFENVTTDAASPPAAQFIGAPPAAELPAPVPAPAPPA
AHEQNAVVATLLFKVEEFYAAANSRPAAKRADVKILESSQVTQQAGGS
SAATATMYPSMVDRKRKRAQATPSRMAPHEVIQYLRATAVETDKELET
LNNFFKVCDGEDKALITFSVEQIRSIKKKIGRIINKPVTAMSSRRMAR
FIDEIDTIKEEKARVFEEIIKILKNPRRKRENDGSSGSNRKNVGGSSG
GKKKTVGGSSGSKKKNVGRPSAKKAQK
Arabidopsis thaliana RKDs
AtRKD4 (AT5G53040)
(SEQ ID NO: 39)
MSSSKHSSVFNYSALFLSLFLQQMDQNSLHHLDSPKIENEYEPDSLYD
MLDKLPPLDSLLDMEDLKPNAGLHFQFHYNSFEDFFENIEVDNTIPSD
IHLLTQEPYFSSDSSSSSPLAIQNDGLISNVKVEKVTVKKKRNLKKKR
QDKLEMSEIKQFFDRPIMKAAKELNVGLTVLKKRCRELGIYRWPHRKL
KSLNSLIKNLKNVGMEEEVKNLEEHRFLIEQEPDAELSDGTKKLRQAC
FKANYKRRKSLGDDYY
(SEQ ID NO: 40)
CFKENYKRRRAAASVN
(SEQ ID NO: 41)
ARWPHRKMKSLRSLI
synTaRKD1-codon optimised sequence of TaRKD1-
(SEQ ID NO: 42)
ATGGAGATGCAACAACAATACTTCGGGGGGGACGGCGATGCGGACTGG
TTCCATCAACTCGCATTGCTTCCCCCACTTCCAATCTCATCGTCTCTC
CCCCCACTCCCGATGTCAGAGGGCTCATGTCTCCCTATGGCAGCAGCA
GCTGCAGCTGCACTCCCCCTTGGCGATTGCTCGAGCGCCCTCATGATA
CGCCCTGAGGAACAGATGTCTTGCCTTCCAATGAACCCCTCTCCAGCG
GTCGTCGACGATGTCTACTCTTCCTACGCACCGAACAATGTCGACGTG
TTGCCGCCATTCCCGGCAGGACTTGACGACGCTCTGTTGATGGAGTCT
TTTTCTGACATCGACCTCGAGGAGTTTGCTGACGCATTTGGCCACAAG
ATCAAGACAGAACCCCTCGACGATGCCATGGTCCCCGCGGACCACGAC
TTCGCGGCTCAAGCCCAACAGGCCTGCCCTGTGGTCATCATGAATCAG
CAACAACTCAACGCACCCAGAGACGTGCGCCTGCTCATTGACCCGGAT
GATGATGACAGCACCGTGGTGGCCGGGGGCTATGAAGCTGCAGCGGTG
GGGTGCGCCGAGCAGAAACAGGTCAGGCCAGCACCACGTAGGGTGAGA
AAGAGCTCAGGCGGCGCAAGACCAGCCGCGGGAGGAAAGTCCCTCGAT
CACATCGGATTCGAGGAACTCAGGACCTATTTCTATATGCCAATCACC
AAGGCAGCGAGGGAAATGAACGTGGGGCTGACAGTCCTGAAGAAGAGA
TGCCGGGAACTGGGGGTGGCGCGCTGGCCACACAGAAAGATGAAGTCT
CTGAGAAGCCTGATCCTCAACATTCAGGAGATGGGGAAGGGCGCAACA
TCTCCCGCAGCCGTGCAGGGGGAACTTGAAGCGCTTGAGAGGTATTGC
GCCATTATGGAGGAGAACCCGGCTATAGAGCTCACCGAGCAAACGAAG
AAGCTCAGGCAGGCTTGTTTCAAAGAGAATTATAAGCGGCGTAGAGCC
GCCGCTTCTGTTAATCTTCTCGATCACTGCTATAACGATCTGGCATCT
CATGAGCAGCAAATGCCTCTCCCACAAATGGGATTCTTTGGATTTTAG
synOsRKD3-codon optimized sequence of OsRKD3-
(SEQ ID NO: 43)
ATGGAAATGCACGAGTGCTGCTACTACGGGGGTTCCATCGGAGGCGAC
TGGCTTAATCCGCTCGCGGCAATACCGCCGCCGTGTTCATCATCCAGC
TCATCGTGGTCTAGCCAGCTGCTGCTGCTCTCGGACCATGACGATGTG
TTGCTCCACAGCGCCGGCGATCACGGAGGGGCGGTGGCGGGAATTGGC
GGTGCCTGTATGACTGCTGATCTGGTGGTGAGGGACGAGGAGATGGAG
ATGGCCGCCGGCTACCTCCCTGTCGCTGCGTCCGCAGCCGCGGCGGCG
GATGTCGATCACTACATGTACCAGCAGTTCCAGCTTGAGCCCGATCAG
TTCGTGTCAACCTTGCCGGCTGTTGCCGTTGCCGTGGCCGCGACAGCA
GGGGGAGGATCACACGACGACGAACTCCTCAGGATGCCATTCACCGAC
ATCGATTTGGATGCATTTGCCGATGCCAGAGATGTGGTCGTCGGAGTT
GGGGAGCCGAAGCCCTCTCCCCAGCATACCCTTGACGCTGCAATCGCG
CTTCCTGCAGTAGGCGGCGGCGGCGCTCATCATTTCGGAACGCAAGAC
GATGACGTGAAGTTCGACGTAACCAAACAGCGGAACGATGCTGCGCTC
GCTGGCGATGATAGTCTGAGTATGGTGATCGTGGAGTCGTACGAGATG
GGAATGAGAAGACATGCGGCTGAGCAAGAGCAGGAACAGAAGCCAAAG
ATCATTACCTCAGCAGCCACTACCCTCACGCCGCTCCCGCTGCCGCCT
CCTCCTCCACCACCACCTAGAGTGACGCGGTCAAGGAGAGATGGTAGC
TCAGCAGCAACCGCCGGGGGAAAAACGCGCCTTGACCACATAGGATTC
GAGGATCTTAGAAGATATTTTTACATGCCCATCACAAAGGCAGCGCGC
GAGATGAATGTGGGGCTTACAGTCTTGAAGAAGAGGTGCAGAGAACTG
GGCGTTGCGCGGTGGCCACACCGCAAAATGAAGAGCTTGAAAAGCTTG
ATCCTTAATGAAATGGGGAGCAAGGGTATGTCTGCTGCCGCGATGAGG
CGCGAGCTGGAAGCCCTTGAAAACTGCTGCGCACTGATGGAGAGGAAC
CCCGCGGTGGAACTTACAGAACGGACTAAGAAGTTGCGCCAGGCCTGC
TTCAAGGAAAATTATAAGCGCAGGCGCGCGGCTGCTGTCGACGTGCTG
GATCTCGACCACTGCTTTTCTTTCGCGGCTGGTCACTGCCACAGACAC
CACCACCAACAGCTTGCGCTGCCCCCACCACCTGCGGCGGCTGCTGAT
CACCGTCGTCGCGATTTTTTCGGCTAC
synAtRKD4-codon optimized sequence of AtRKD4-
(SEQ ID NO: 44)
ATGTCCTCATCGAAGCACTCATCTGTTTTCAATTATTCTGCCTTATTT
CTTAGTCTGTTCTTGCAGCAAATGGATCAAAATAGCTTACACCACCTG
GACTCACCGAAGATTGAGAATGAGTACGAACCGGACTCCTTATATGAC
ATGTTAGATAAGCTGCCTCCTCTGGACTCACTTTTAGACATGGAAGAC
CTGAAGCCAAACGCCGGACTTCACTTCCAATTCCACTATAACAGCTTT
GAAGACTTTTTTGAGAATATCGAAGTAGACAATACCATTCCTAGTGAC
ATCCACTTACTGACACAGGAACCGTACTTTTCGAGTGATTCCAGCAGT
TCGTCCCCGCTGGCCATTCAAAATGACGGCTTAATCTCCAATGTAAAA
GTGGAAAAGGTCACCGTGAAAAAGAAGCGTAATCTGAAAAAAAAACGC
CAAGATAAGTTAGAAATGTCGGAGATCAAGCAGTTCTTCGACCGCCCC
ATTATGAAGGCGGCGAAGGAGCTGAATGTGGGTTTAACTGTATTAAAA
AAGCGCTGTCGTGAGTTGGGCATCTATCGCTGGCCTCACCGTAAACTT
AAGTCTCTTAACTCCCTGATTAAAAATTTGAAGAACGTGGGGATGGAG
GAAGAGGTAAAGAATTTGGAGGAGCACCGCTTCCTTATTGAACAGGAG
CCGGATGCTGAGCTTTCTGATGGGACCAAGAAACTGCGTCAAGCCTGC
TTCAAAGCTAATTACAAGCGCCGCAAGTCCTTGGGGGATGATTACTAC

### BIBLIOGRAPHY

Alseekh S, Scossa F, Wen W, Luo J, Yan J, Beleggia R, Klee H. J., Huang S, Papa R, Fernie A. R. Domestication of Crop Metabolomes: Desired and Unintended Consequences Trends in Plant Science, 2021, 26 (6), 650-661, ISSN 1360-1385. doi.org/10.1016/j.tplants.2021.02.005.
Koi S, Hisanaga T, Sato K, Shimamura M, Yamato K T, Ishizaki K, Kohchi T, Nakajima K. An Evolutionarily Conserved Plant RKD Factor Controls Germ Cell Differentiation. Curr Biol. 2016-7-11; 26 (13): 1775-1781. doi: 10.1016/j.cub.2016.05.013. Epub 2016-6-23. PMID: 27345165.
Mitchell C. Hunter, Richard G. Smith, Meagan E. Schipanski, Lesley W. Atwood, David A. Mortensen, Agriculture in 2050: Recalibrating Targets for Sustainable Intensification, BioScience, Volume 67, Issue 4, April 2017, Pages 386-391, https://doi.org/10.1093/biosci/bix010
United Nations Population Division, World population prospects: 2019 Highlights, United Nations, ISSN: 9789211483161

1. A method of producing a modified plant, part, cell or protoplast thereof having an increase in a yield-related trait relative to a reference plant, part, cell or protoplast thereof, comprising:
(a) Providing the plant, part, cell or protoplast thereof with an RKD transcription factor or a variant or active fragment thereof, or a polynucleotide encoding said RKD transcription factor, variant or active fragment thereof; or
(b) Providing the plant, part, cell or protoplast thereof with a gene editing system for modifying an endogenous nucleic acid sequence encoding an RKD transcription factor or a regulatory sequence controlling expression thereof;
(c) Obtaining a modified plant, part, cell or protoplast thereof having an increase in RKD transcription factor expression; and
(d) Optionally regenerating a modified plant from the or each plant part, cell or protoplast.

2. The method according to claim 1, wherein step (a) comprises introducing the RKD transcription factor protein or a variant or active fragment thereof, or a polynucleotide encoding said RKD transcription factor, variant or active fragment thereof into the plant, plant part, cell or protoplast, preferably wherein introducing comprises transforming or transducing the plant, plant part, cell or protoplast thereof with the RKD transcription factor or a variant or active fragment thereof, or a polynucleotide encoding said RKD transcription factor, variant or active fragment thereof.

3. The method according to claim 1 or 2, wherein the RKD transcription factor comprises an amino acid sequence comprising the motif RWPHRK (SEQ ID NO: 2), preferably wherein the RKD transcription factor is a plant-specific RWP-RK transcription factor comprising an amino acid sequence comprising the motif RWPHRK (SEQ ID NO: 2).

4. The method according to claim 1, 2 or 3, wherein the RKD transcription factor comprises an amino acid sequence according to any one of SEQ ID NO: 1, 4, 6-39 or a functional variant thereof.

5. The method according to any of claims 1 to 4, wherein the RKD transcription factor is a reproductive RKD transcription factor.

6. The method according to claim 5, wherein step (a) comprises providing a vegetative tissue of a plant or plant part, or a vegetative cell or protoplast thereof with the reproductive RKD transcription factor or a variant or active fragment thereof, or a polynucleotide encoding said reproductive RKD transcription factor, variant or active fragment thereof.

7. The method according to claim 5 or 6, wherein step (a) comprises ectopically expressing the reproductive RKD transcription factor or a variant or active fragment thereof, or a polynucleotide encoding said reproductive RKD transcription factor, variant or active fragment thereof in a vegetative tissue of a plant, plant part, cell or protoplast.

8. The method according to any of claims 5 to 7, wherein the reproductive RKD transcription factor is selected from RKD1, RKD2, RKD3, RKD4, and RKD10, preferably wherein the reproductive RKD transcription factor is selected from TaRKD1, TaRKD3, TaRKD4, TaRKD10, AetRKD1, AetRKD3, and AetRKD4.

9. The method of claim 8, wherein the reproductive RKD transcription factor is selected from the group consisting of: TaRKD1-7a, TaRKD1-7b, TaRKD1-7d; TaRKD3-7a, TaRKD3-7d; TaRKD4-6a, TaRKD4-6b, TaRKD4-6d; TaRKD10-7a, TaRKD10-7d; AetRKD1-7d; AetRKD3-7d and AetRKD4-7d.

10. The method of any of claims 1-4, wherein the RKD transcription factor is a vegetative RKD transcription factor.

11. The method according to claim 10, wherein the vegetative RKD transcription factor is selected from RKD6a, RKD6b, RKD9, and RKD11, preferably wherein the vegetative RKD transcription factor is selected from TaRKD6a, TaRKD6b, TaRKD9, TaRKD11, AetRKD6a, AetRKD6b and AetRKD9.

12. The method according to claim 11, wherein the vegetative RKD transcription factor is selected from the group consisting of: TaRKD6a-2a, TaRKD6a-2b, TaRKD6a-2d; TaRKD6b-2a, TaRKD6b-2b; TaRKD9-3a, TaRKD9-3b, TaRKD9-3d; TaRKD11-7a; AetRKD6a-2d; AetRKD6b-2d and AetRKD9-3d.

13. The method according to claim 1, wherein the RKD transcription factor is TaRKD1, or TaRKD4, preferably wherein the RKD transcription factor is selected from: TaRKD1-7d (SEQ ID NO: 6), TaRKD1-7a (SEQ ID NO: 1) and TaRKD1-7b (SEQ ID NO: 4, preferably wherein the RKD transcription factor is TaRKD1-7d (SEQ ID NO: 6).

14. The method according to any preceding claim, wherein the yield-related trait is selected from one or more of: (i) number of axillary meristems; (ii) number of tillers; (iii) rachis length; (iv) spike length; (v) number of spikelets per spike; (vi) number of grains per spike; (vii) panicle branch number; (viii) seed weight and/or number of seeds; and/or (ix) thousand kernel weight.

15. The method of any preceding claim wherein the plant is a crop plant, part, cell or protoplast thereof, preferably wherein the plant is selected from: wheat (Triticinae), rice (Oryza), barley (Hordeum), oat (Avena), maize (Zea) or sorghum (Sorghum), or parts, cells or protoplasts thereof.

16. A modified plant, part, cell or protoplast thereof obtained by the method of any of claims 1-15.

17. A modified plant, part, cell or protoplast according to claim 16, which comprises an increase in a yield-related trait relative to a reference plant, plant part, cell or protoplast.

18. A seed, other plant part, or material derived from the modified plant of claim 16 or 17.

19. Use of an RKD transcription factor or a variant or active fragment thereof, or a polynucleotide encoding said RKD transcription factor or a variant or active fragment thereof, in the production of a plant, part, cell or protoplast thereof which has an increase in a yield-related trait relative to a reference plant, part, cell or protoplast thereof.

20. Use of an RKD transcription factor or a variant or active fragment thereof, or a polynucleotide encoding said RKD transcription factor or a variant or active fragment thereof, or an associated marker thereof, as a selectable marker in plant transformation.

21. The use according to claim 20 wherein the marker is used for selecting plants having an increase in a yield-related trait.

22. A method of selecting a plant comprising an increase in a yield-related trait relative to a reference plant, comprising:
(a) Providing a plant;
(b) Optionally mutagenizing the plant;
(c) Measuring the expression level of at least one RKD transcription factor or an associated marker thereof in at least one cell of the plant;
(d) Comparing the measured expression level to a reference expression level of the same RKD transcription factor or an associated marker thereof in a reference plant; and
(e) Selecting the plant if the measured expression level is increased relative to the reference expression level.

23. The method according to claim 22 wherein the plant was produced or obtained by the method of any of claims 1-15, or is a plant of claim 16 or 17.

24. A method of plant improvement comprising combining the genetic material of a first plant obtained by the method with the genetic material of a second plant.

25. The method according to claim 24, wherein the second plant is a plant obtained by the method of any of claims 1-15, or is a plant of claim 16 or 17, or is another plant differing in genotype from the first plant.

26. The method according to claim 24 or 25, wherein progeny are produced which comprise increased expression of an RKD transcription factor thereof relative to a reference plant.

27. The method according to any of claims 24-26, wherein the method is a method of improving plants such that they have an increase in a yield-related trait relative to a reference plant.

28. A method of producing a hybrid seed comprising crossing a first modified plant obtained by the method with a second plant; and obtaining a seed.

29. The method according to claim 28, wherein the second plant is a plant obtained by the method of any of claims 1-15, or is a plant of claim 16 or 17, or is another plant differing in genotype from the first plant.

30. The method according to claim 28 or 29, wherein the seed comprises a genotype which confers an increased expression of an RKD transcription factor thereof relative to a reference plant.

31. A hybrid seed obtained by the method of any of claims 29-30.

32. A hybrid plant generated from the hybrid seed of claim 31.


SEQUENCE LISTING (44 sequences)

SEQ ID NO: 1 - protein, 348 aa, Triticum aestivum
  REGION 1..348: note = TaRKD1-7A
MEMQQYFGGCGDGDADWFHQLALLPPLPVSSSLPPLPMRPEEQMGCLQMIPPQAVADDEYSSYATNNVDVLPPFPAGLDDPTAGLDDALLMESFRDIDLEEFADAVGPKIKTEPLDDAMVPADHDFAAQVQQARPVVIMNQQQLNAPHGVRLLNDPDDDDSAVVAGGYEAAAVGCAEQKQVRPAPRRVRKSSGGSRPAAGGKSLDHIGFEELRTYFYMPITKAAREMNVGLTVLKKRCRELGVARWPHRKMKSLRSLILNIQDMGKGATSPAAVQGELEALERYCAIMEENPAIELTEQTKKLRQACFKENYKRRRAAASVNLLEHCYNDLGSHEQQMPLPQMGFFGF

SEQ ID NO: 2 - protein, 6 aa, synthetic construct
  REGION 1..6: note = Conserved RKD motif
RWPHRK

SEQ ID NO: 3 - protein, 87 aa, synthetic construct
  REGION 1..87: note = Conserved RKD motif and flanking regions
KSSGGSRPAAGGKSLDHIGFEELRTYFYMPITKAAREMNVGLTVLKKRCRELGVARWPHRKMKSLRSLILNIQDMGKGATSPAAVQG

SEQ ID NO: 4 - protein, 376 aa, Triticum aestivum
  REGION 1..376: note = TaRKD1-7B
MEMQQYYGGGGGCDGDGDWFHQLAALPPLPMSSSLPPPLLMSEGSCLPMAAAAALPLGDCSSALMIRPEEQMGCLQMIRPPAVADDVYSSYGPNNVDVLPPFPAGLDDPTAGLDDALLMESFSDIDLEEFADAVGHKIKTEPLDDAMVPADHDFAAQAQQACPVVIMNQQQLNAPRNVRLLKDPDDNDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRATASVNLLEHCYNDLGSHEQQMPLPQMGFFGF

SEQ ID NO: 5 - protein, 87 aa, synthetic construct
  REGION 1..87: note = Conserved RKD motif and flanking regions
KSSGGARPAAGGKSLDHIGFEELRTYFYMPITKAAREMNVGLTVLKKRCRELGVARWPHRKMKSLRSLILNIQEMGKGATSPAAVQG

SEQ ID NO: 6 - protein, 367 aa, Triticum aestivum
  REGION 1..367: note = TaRKD1-7D
MEMQQQYFGGDGDADWFHQLALLPPLPISSSLPPLPMSEGSCLPMAAAAAAALPLGDCSSALMIRPEEQMSCLPMNPSPAVVDDVYSSYAPNNVDVLPPFPAGLDDALLMESFSDIDLEEFADAFGHKIKTEPLDDAMVPADHDFAAQAQQACPVVIMNQQQLNAPRDVRLLIDPDDDDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRAAASVNLLDHCYNDLASHEQQMPLPQMGFFGF

SEQ ID NO: 7 - protein, 248 aa, Triticum aestivum
  REGION 1..248: note = TaRKD3-7A
MDSSGEHSFWPYGPSVWHDSYLLEEETLLCTLPFPSYGCQPLCSISPAVAQSSSLQAPCLVPCDTDELDTSFDDDLLRCWEAIEQSDDSTEESGKGLPLLCYGEVNGAASNAMRADRVRSERVLTFELVSQYFYMPITQAARELNVGLTVLKKRCRELRIPRWPHRKLKSLQTLINDVEVLQEAGKANDDGQLRAMVEMLEQERRLLEQRPYVELEEKTKRLRQACFKASYKKRRLLALEPGEASKYY

SEQ ID NO: 8 - protein, 256 aa, Triticum aestivum
  REGION 1..256: note = TaRKD3-7D
MFQISLERMDSNGEHSFWPYGPSVWHDSYLLEDEALLCSLPFLSFGCQPLCSNSPAVAQSSSLQAPCLVPCDTGELDTSFDDDLLRCWEAIEQSDDSTEDSGKGLPLLCYGEESGAASNAMRADRVRSERVLTFELVSQYFYMPITQAARELNVGLTVLKKKCRELGIPRWPHRKLKNLQTLINDVEVLQEAGKANDGEQLRAMVEMLEQERRLLEQRPYVELEEKTKRLRQACFKASYKKRRLLVLEPGEASKYY

SEQ ID NO: 9 - protein, 204 aa, Triticum aestivum
  REGION 1..204: note = TaRKD4-6A
MYLLRQWSFEYLLEISLFYPTHRLCDTSTSSTTTATQSCPLQDQQEFEARLEVHIMSLLAALEGEHHGGEKELKGEVGRDAGEKKRDGPEEKPLLLFQQVSRCFCMPIKQAAEELNVGLTLLKRQCRELGIPRWPHRKLKSLETLIKNAQELGKPMIEVEMLQRKKKLIEERPGHIELDEETKVLRQACFKEKFKRRRLMAMER

SEQ ID NO: 10 - protein, 204 aa, Triticum aestivum
  REGION 1..204: note = TaRKD4-6B
MYLLQQWSFEYLLEISLFYPTQHLCDTSTSSTTTTSQSYLLQDQQELEARLEVHIMSLLAALEGEHHGGEKVLKGEVGRDAGEKKRNGPEEKPLLSFQQVSQCFCMPIKQAAEELNVGLTLLKRQCRELGIPRWPHRKLKSLETLIKNAQELGKPMVEVEMLQRKKKLIEERPGHIELDEETKVLRQACFKEKFKRRRLMAMER

SEQ ID NO: 11 - protein, 205 aa, Triticum aestivum
  REGION 1..205: note = TaRKD4-6D
MYLLRQWSFEYLLEISLFYPTQHLCDTSTSSTTTTTSQSCLLQDQQELEARLEVHIMSLLAALEGEHHGGEKVMKGEVGRDAGEKKRNGPEEKPLLSFQQVSRCFCMPIKQAAEKLNVGLTLLKRQCRELGIPRWPHRKLKSLETLIKNAQELGKPMIEVEMLRRKKKLIEERPGHIELDEVTKVLRQACFKEKFKRRRLMAMEG

SEQ ID NO: 12 - protein, 248 aa, Triticum aestivum; identical to SEQ ID NO: 7
  REGION 1..248: note = TaRKD10-7A

SEQ ID NO: 13 - protein, 256 aa, Triticum aestivum; identical to SEQ ID NO: 8
  REGION 1..256: note = TaRKD10-7D

SEQ ID NO: 14 - protein, 345 aa, Triticum aestivum
  REGION 1..345: note = TaRKD6a-2A
MDAAAAAVTTLSALAVFASTLHQGAVRSVHGYRVVAMGGRCAWDRWVEREFAFSPTSCREVPLPAAAPRILPVEWRGRPAYREGQVLGAWRCVLAFDSIAAVPLPPTPPPMLCPFRNPGLVCVPSLYNDLYMVYQFQKFQQAPELVKRDSDEKSTRLEADDKTSDVEVKEESGSDSDEDPQSGEELPAHVERRRRAKKQHIASITLADIAPYFHLPIREASRALKIGVSILKQKCRQYGIPRWPHRKIKSLDSLISDLEFVMDDTDGDVVQKETRRQREKEREKEKEKEKEKQEAIQALAKRKRLLESEKEIIQQKPALDLMAETKLFREDVFKRRYRAKRLPGQ

SEQ ID NO: 15 - protein, 355 aa, Triticum aestivum
  REGION 1..355: note = TaRKD6a-2B
MVEYGYKFSSKKIKQELRYTCISTFRFNFLAFCIASMDAAAAAAVTTLSALAVFASPLHQGAVRSVHGYRVVAMGGRCAWDRWVEREFAFSPTSCREVPLPAAAPRILPVEWRGRPAYREGQVLGAWRCILAFDSIAAVPLPPMPPPMLCPFRNPSLVFVPSLYNDLYMVFQFQKFQQAPELVKRDSDEKSTRLDAEDKTSHAERRRRAKKQHIASITLVDIAPYFHLPIREASRALKIGVSILKQKCRQYGIPRWPHRKIKSLDSLISDLEFVMDDTDGDVVQKETRRQREKEKEEKEKEKQEAIRALAKRKRLLESEKEIIQQKPALDLMAETKLFREDVFKRRYRAKRLPGR

SEQ ID NO: 16 - protein, 350 aa, Triticum aestivum
  REGION 1..350: note = TaRKD6a-2D v1
MDAAAAAAVTTLSALAVFASTLHQGAVRSVHGYRVVAMGGRCAWDRWVEREFAFSPTSCREVPLPAAAPRILPVEWRGRPAYREGQVFGTWRCVLAFDSIAAVPLPPTPPPMLCPFRNPGLVCVPSLYNDLYMVYQFQKFQQVPELVKRDSDEKSTRLDAEDKTCDVEVKEESGSDSDEDPQSGEELPAHVARRRRAKKQHIASITLADIAPYFHLPIREASRALKIGVSILKQKCRQYGIPRWPHRKIKSLDSLIGDLEFVMDDTDGDVVQKETSRQREKEREKEKEKEKEKEKEKQEAIQALAKRKRLLESEKEIIQQKPALDLMAETKLFREDVFKRRYRAKRLPGH

SEQ ID NO: 17 - protein, 301 aa, Triticum aestivum
  REGION 1..301: note = TaRKD6a-2D v2
MGGRCAWDRWVEREFAFSPTSCREVPLPAAAPRILPVEWRGRPAYREGQVFGTWRCVLAFDSIAAVPLPPTPPPMLCPFRNPGLVCVPSLYNDLYMVYQFQKFQQVPELVKRDSDEKSTRLDAEDKTCDVEVKEESGSDSDEDPQSGEELPAHVARRRRAKKQHIASITLADIAPYFHLPIREASRALKIGVSILKQKCRQYGIPRWPHRKIKSLDSLIGDLEKETSRQREKEREKEKEKEKEKEKEKQEAIQALAKRKRLLESEKEIIQQKPALDLMAETKLFREDVFKRRYRAKRLPGH

SEQ ID NO: 18 - protein, 337 aa, Triticum aestivum
  REGION 1..337: note = TaRKD6b-2A
MEAPAAAAAAASTLSALAVFVSTVDRGAVRSAHGYRVAGKGGGCGWERWVEREFAFTPTLAREVPLPADALRLLPADWRGRPVYREGQIAGPWRCILAFDFVAAVAPPPVPPPVLCPFRNPRLMCVPSLYNDLTKVFQFQKVEEKVPGLVQCDSDEKLTTSDAKDRVSVKQEAGSDTDEPPQFGEDLLAPAEKQRRAHREYIASITLEDITQYFHLPIREASRTLKIGLSILKKKCRQYGIPRWPHRKLKSLDSLIHDLEFVIDDETERDGVKQEDHMQDEKENQDAIEALAKRKRMLETEKATIQQKPTLDLMAETKQFRQYVFKRKYRAKTLVSE

SEQ ID NO: 19 - protein, 337 aa, Triticum aestivum
  REGION 1..337: note = TaRKD6b-2B
MEAPAAAAAAVSTLSALAVFVSTVDRGAVRSAHGYRVAGKGGGCGWERWVEREFAFTPTSAREVPLAADAQRLLPADWRGRPVYREGQIAGPWRCILAFDFVAAVAPPPVPPPVLCPLRNPRLMCVPSLYNDLTKVFQFQKVEEKVPGLVQCDSDEKLTSSDAKDRVSVKQEAGSDTDERPQFGEDLLAPAEKQRRAHRAYIASITLEDIAQYFHLPIREASRTLKIGLSILKKKCRQHGIPRWPHRKLKSLDSLIHDLEFVIDDETERDGVKQEDHMQDEKENQDAIEALAKRKRMLETEKATIQQKPTLDLMAETKQFRQYVFKRKYRAKTLVSE

SEQ ID NO: 20 - protein, 306 aa, Triticum aestivum
  REGION 1..306: note = TaRKD9-3A
MSAPPMAASAQELPPAQQQPAAEQAPSLALALVPQPQAQAQIKPTRVSLSYEEISKLFSLPIAEAASILGVCTSVLKRICRTHGIVRWPYRKIVSGKGDDAKNAEREKAMQLLELSKIAKQKAISSSGSLATSSSGAFQGVPKAQQGSAKAGTAIGRQNAPSLSQFSQAKDIPTYMDDFKYGFPSSGLSTETMKWWGTDSHTETAPAKDDNREGSESTNEASKGTDDELDWGADEPEADADGVAAATDPSAQLCSLRRKAAGDGRRLLNGDTGRVQQLCRLNKRQKIVLAQVFGASLPEQRRSKLA

SEQ ID NO: 21 - protein, 301 aa, Triticum aestivum
  REGION 1..301: note = TaRKD9-3B
MSAPPMAASAQELPPAEQAPSLALALVPQPQAQAQIKPTRVSLSYEEISKLFSLPIAEAASILGVCTSVLKRICRTHGIVRWPYRKIVSGKGDDAKNAEREKAMQLLELSKIAKQKAISSSGSLATSSSGAFQGVPKAQQGSAKAGTAIGRQNAPSLSQFSQAKDIPTYMDDFKYGFPSSGLSTETMKWWGTDSHTETAPAKDDNREGSESTNEASKGMTDDELDWGADEPEADADGVAAATDPSAQLCSLRRKAAGDGRRLLNGDTGRVQQLCRLNKRQKIVLAQVFGASLPEHWRSNLA

SEQ ID NO: 22 - protein, 302 aa, Triticum aestivum
  REGION 1..302: note = TaRKD9-3D
MAASAQELPPAQQQPAAEQPPSLALALVPQPQAQAQIKPTRVSLSYEEISKLFSLPIAEAASILGVCTSVLKRICRTHGIVRWPYRKIVSGKGDDAKNAEREKAMQLLELSKIAKQKAISSSGSLATSSSGAFQGVPKAQQGSAKAGSAIGRQNAPSLSQFSQAKDIPTYMDDFKYGFPSSGLSTETMKWWGTDSHTETAPAKDDNREGSESTNEASKGMTDDELDWGADEPEADADGVAAATDPSAQLCSLRRKAAGDGRRLLNGDTGRVQQLCRLNKRQKIVLAQVFGASLPEQWRSKLA

SEQ ID NO: 23 - protein, 369 aa, Triticum aestivum
  REGION 1..369: note = TaRKD11-7A
MLMATGDGADGLGLDDDSLPLQDNFDDVLQLLDDFPDPDTAPIPLPAQMHHADQPACSHSFSGDGLRNNSTGDGTPGVAQPNEFPVHSDPSTSSTAAGVLDCSGCHVLREVMHSNGPELTKLSIHGAPGFFNHATLDRCTNSEGLAPRIAPRSHIDFRDRDYEWVGHFLTDYALRQAAGNYAVVRDSLSLFLDVLCTTMNRCVQGNDGDHDDEGTAARNGGHGQPTVDVFAAAVQPAMQRNTEPEPAGPSPSPPSNTMELHVQPQPFQPVVAGRSVLALQRERTRKMQFHDIAPYFHLPIVEAAEKLDICTTVLKGICRRVGVQRWPHRKVKKIDRQITKLMRSGNGVWERNEIERLNAERKRIFYALE

SEQ ID NO: 24 - protein, 374 aa, Aegilops tauschii
  REGION 1..374: note = AetRKD1-7D
MEMQQQYFGGDGDADWFHQLALLPPLPISSSLPPLPMSEGSCLPMAAAAAAALPLGDCSSALMIRPEEQMSCLPMNPSPAVVDDVYSSYAPNNVDVLPPFPAGLDDPTAGLDDALLMESFSDIDLEEFADAVGHKIKTEPLDDAMVPADHDFAAQAQQACPVVIMNQQQLNAPRDVRLLIDPDDDDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRAAASVNLLDHCYNDLASHEQQMPLPQMGFFGF

SEQ ID NO: 25 - protein, 256 aa, Aegilops tauschii; identical to SEQ ID NO: 8
  REGION 1..256: note = AetRKD3-7D

SEQ ID NO: 26 - protein, 205 aa, Aegilops tauschii; identical to SEQ ID NO: 11
  REGION 1..205: note = AetRKD4-7D

SEQ ID NO: 27 - protein, 304 aa, Aegilops tauschii
  REGION 1..304: note = AetRKD6a-2D
MGGRCAWDRWVEREFAFSPTSCREVPLPAAAPRILPVEWRGRPAYREGQVFGTWRCVLAFDSIAAVPLPPTPPPMLCPFRNPGLVCVPSLYNDLYMVYQFQKFQQVPELVKRDSDEKSTRLDAEDKTCDVEVKEESGSDSDEDPQSGEELPAHVARRRRAKKQHIASITLADIAPYFHLPIREASRALKIGVSILKQKCRQYGIPRWPHRKIKSLDSLISDLEVNLKETSRQREKEREKEKEKEKEKEKEKQEAIQALAKRKRLLESEKEIIQQKPALDLMAETKLFREDVFKRRYRAKRLPGH

SEQ ID NO: 28 - protein, 214 aa, Aegilops tauschii
  REGION 1..214: note = AetRKD6b-2D
MCVPSLYNDLTKVFQFQKVEEKVPGLVQCDSDEKLTTSDAKDRVSVKQEAGSDTDEPPQFGEDLLAPAEKQRRAHREYIASITLNDIAQYFHLPIREASRTLKIGLSILKKKCRQYGIPRWPHRKLKSLDSLIHDLEYVIDDETERDGVKQEDHMQDEKENQDAIEALAKRKRMLETEKATIQQKPTLDLMAETKQFRQYVFKRKYRAKTLVSE

SEQ ID NO: 29 - protein, 302 aa, Aegilops tauschii
  REGION 1..302: note = AetRKD9-3D
MAASAQELPPAQQQPAAEQPPSLALALVPQPQAQAQIKPTRVSLSYEEISKLFSLPIAEAASILGVCTSVLKRICRTHGIVRWPYRKIVSGKGDDAKNAEREKAMQLLELSKIAKQKAISSSGSLATSSSGAFQGVPKAQQGSAKAGSAIGRQNAPSLSQFSQAKDIPTYMDDFKYGFPSSGLSTETMKWWGTDSHTETAPAKDDNREGSESTNEASKGMTDDELDWGADEPEADADGVAAATDPSAQLCFLRRKAAGDGRRLLNGDTGRVQQLCRLNKRQKIVLAQVFGASLPEQWRSKLA

SEQ ID NO: 30 - protein, 315 aa, Oryza sativa
  REGION 1..315: note = OsRKD1
MCAAPMASTAQPQQQPQQQQEQQPVAAAAVPTPAPPASEAQPQKPTRVSLSYEEISKLFSLPIAEAASILGVCTSVLKRICRSHGIVRWPYRKLVSGKSGDDTKNAEREKAKGLLEISKVAKQKALSASGLSTVSPGAFQGVAKSQQGSSKAGQVSPPGKQNVLGGSAILSYGTQTKGIPTYMDDFKYGFPSSGLSLQTMKWWGTDSHTETTPAKDDNGEAPESANEASKGMTDDELDWGADEAEAEADADSAITTEPSAQLCSLRRKAVDDGRKLLTGKSCGGLELCRLNKRQKMALAQVFGASLPEQLRSKLG

SEQ ID NO: 31 - protein, 441 aa, Oryza sativa
  REGION 1..441: note = OsRKD3
MEMHECCYYGGSIGGDWLNPLAAIPPPCSSSSSSWSSQLLLLSDHDDVLLHSAGDHGGAVAGIGGACMTADLVVRDEEMEMAAGYLPVAASAAAAADVDHYMYQQFQLEPDQFVSTLPAVAVAVAATAGGGSHDDELLRMPFTDIDLDAFADARDVVVGVGEPKPSPQHTLDAAIALPAVGGGGAHHFGTQDDDVKFDVTKQRNDAALAGDDSLSMVIVESYEMGMRRHAAEQEQEQKPKIITSAATTLTPLPLPPPPPPPPRVTRSRRDGSSAATAGGKTRLDHIGFEDLRRYFYMPITKAAREMNVGLTVLKKRCRELGVARWPHRKMKSLKSLILNEMGSKGMSAAAMRRELEALENCCALMERNPAVELTERTKKLRQACFKENYKRRRAAAVDVLDLDHCFSFAAGHCHRHHHQQLALPPPPAAAADHRRRDFFGY

SEQ ID NO: 32 - protein, 339 aa, Oryza sativa
  REGION 1..339: note = OsRKD4
MDAAAAAVSTLSALAVFASTLDHGAVRSVHGYKVYGRGGRRRWERWVEREFVLTPASCREVPAPVAPPRILPAEWRGRPAYREGQVVAAGAWRCILAFDSAAAPPRTPPPVLSPFLNPRLMCVPSLYNDLEKVFRFQNVEKIPKLMQCDSEEKLSSWDARDKSSDEVHASESDSDDDLQSGEEEKPTVQKQRRANKKHIASITLVDIAQYFHLPIREASRTLKIGVSILKRKCRQYNIPRWPHRKIKSLDSLIQDLEYVIDDGDDHDDTGDDVQQEKHKQTAEEKQEAIMALTRRKQMLETEKETIQQIPAMDLKVETKQFREDVFKRRYRAKKDLAND

SEQ ID NO: 33 - protein, 921 aa, Oryza sativa
  REGION 1..921: note = OsRKD5
MRHNRSLAALLRAGRYGAARRLFDALPARSVVTWNSLLAGLARRPDARAAREFFDAMPVRDAVSWNTLLAAYSASPHPDHLAAARRLFDEMPQRDVVTWNTLLGAYARRGLMDEARRLFDEMPQRNAASWNTMVTGFFAAGQVVKALDVFDAMPAKDSASLSTMVSGFTKNGMLHEAEELLTKRLSVTDMDKAVDAYNTLIVAYGQAGRFSDAKRLFDMIPKGQYQHNMLKRKGFERNVVSWNSMMICYIKAGDVCSARALFNEMPDKDLVSWNTMISGYTQASDMKESEKLFWEMPDPDTVSWNLIIQGFMQKGEAEHARGFFDRMPERGTISWNTMISGYEKNGNYISSVKLFSKMLEVGEIPDRHTFSSVLAACASIPMLGLGAQIHQLVEKSFVPDTAISNALITMYSRCGALNDAEAIFKQMHTKKDLVSWNALIGCYEHHGRATKALQLFKEMRRAKVMPTHITFVSLLSACVNAGLVSEGRMVFDTMVHEYGIVARIEHYAALVNLIGRHGQLDDALEVINSMPMAPDRSVWGAFLGACTAKKNEPLAQMAAKELSTINPDSSAPYVLIHNLHAHEGKWGSAAVVREEMERQGIYKQPGYSWIDLEGKMHVFISGDTWHPNAQEIFSVLEDWQWHNPMSLEMNQLTQVKLIAKRNNQIMDKQATATKMHKATNIRHIYSKFKVDEQQNTSWWPYCTSLWPDSYLLEEEALFSSLSFPSFHPQPVYSTVMQSNVLQDELGVIFEDDVLKYWDEMEQSENKVEKSEKGLPLLYYGDENGAASKIMRDDVRSEEKALTFELVSQYFYMPITQAARELNVGLTLLKKKCRELGIPRWPHRKMKSLQTLINNVQVLQEASKANNEEQLRMLVEMLQEERRLLEQKPYVQLEEKTKRLRQACFKANYKKRRLLALEAGEP

SEQ ID NO: 34 - protein, 232 aa, Oryza sativa
  REGION 1..232: note = OsRKD6
MEKWRFYGGHQFQSYSFHEEENLFQDWSLDYLLLGEDEPFFTHHFSTSVHSNFVQDELYTLFDGDILSIWGDMKEDAYHRSDKDGGEKEEKLDHEKAMELQLQRLPSGRQSGEKTLTFELVSQYFCLPIKQAAQELNVGLTLLKRRCRVLGIPRWPHRKVKSLETIIKNVQELGMETGQDEDNTRNAVEMLQQTKKLIEQSPDAKLDDWTKMLRQACFKENYKRRRLLAIEG

SEQ ID NO: 35 - protein, 495 aa, Oryza sativa
  REGION 1..495: note = OsRKD7
MAMSRATCSTRPRISRCSPSRRWFVPVAAAIESHPTPAIHVVEREWPAQVQISRFDPCSPPPPARMLPGGQQQQPHPAPGGLDLRSDEIHALKQRRRPPPNPQSSSPPEGLLGAANPDRPDDDELDAILSSFRDEAAHGDSGGGVARVQVLPVEQDANLVPSLPLRHGQLDCSRCHLVRHVMHVAVLPYVKFSLKLVWEEAFHRIRRMYVISVRSNVYSLSKWTQEWASEFIARNIDTMRNNTNGQLLDSGYSNLVESVRTNVNVPHTAVEVNLLQTIMSAPSADHHQNAADQVAAPAAQPFSAAPPVALPPKAAPRKARKDRDYASMLVAVEEFYVAATSRPVPNSDVEILESSHVSQQQDGGRAIIYPSLQARRGKTKQEVPRRNAKDVLEYLSLARKETEKEINTLSSFDGIYRNDGTLSYLMTEVRRLNRKIWRLQKNAPSTLSSRLLASVKEIDDIKVEKGRLYAQFISALKKLCRKKMDDGGSAPSANN

SEQ ID NO: 36 - protein, 452 aa, Oryza sativa
  REGION 1..452: note = OsRKD8
MAGDGGNGAADGGGEHLELDFFPSPLLDNFDVLELLDLSVLGDVDDDGAPPAPAPVQMSVDGAGGGRINKPGSSVDDLVDWSTAFGNNCSKIDGEYGGASTSSSAAPALAPPQEDYCSGCQVLREVVHSNGLEITKLCIHGGVASGEFYHAILDVYRVSASAPAPALAHHSIINFRGRGYDWVKQYLTEYALRRAGGGFAVVQDSLSAFHDALCTTMAPCSSHVGDDDAHRRASSSAAAAAEERTNGNGDHGQLVVHNAAVLPMLESSRCLVAADQAATTNNNGSGDRRLVVLDTTAIQPPASGCILHMAFPSKSYQSSRPTTLPSRYQECTCNPVAYEMDGVRSSLAPICWPELLGYNVTNREKTKQLQLSDIAPYFELPIAKAAKKLDICATALKGICRKHGVLRWPYRKVRSIDRQIATLRRSGNGDATRNEIETLIASRRRIVAGLDQ

SEQ ID NO: 37 - protein, 339 aa, Oryza sativa
  REGION 1..339: note = OsRKD9
MATGGSSSSSNVPSGGAAGGGAGSGKLPMVPVGSIECLRKRCRQLRVLIHVNDHRKAVVVLHAGEDGKPDHVLVQNVSPEGEASVQSTYRIDVSGETPESQAEMLNDWYTSFRMDTTGVLYDSDQNVIYGVPRGHPGGDVPRSLAILPPAPKKNQHGKAPATESNSSLVEEPLLLVQTDQPAAIGKRKKFTFPDQRKRVKTMTKKDLESYFHITQKSAAHIGLSIGTTALKNLCRANDLPRWPYRQIASLDNKFNNNLKKQITGWNLGKAVQGVTKAFKLRKEKEEFYQKIMSSMPEQLQSIDEIVNSLPEADDDIDIEDDEDNDDVIEDNDDDNSDEN

SEQ ID NO: 38 - protein, 603 aa, Oryza sativa
  REGION 1..603: note = OsRKD10
MAGEHRFYDSNGDVSRYMLDDMEFLPLFGEAEAATTLPLPVLPDVEPLPTAPAPAATAHVEPAPAADFGLGNPVLADLGFDVDLDFFPELNFQSPPPPPAMNAGGYTHQVQASPPVMHHQQQQQQPLAPLPAAHGFAGQPAPATTMAPSGGDDGLFLAAPSRDAPCSPVMFNFMDFNVDMGDVDMDDVLMWADQDTHGAAAGGDTAPPVVVDEYADFVPFQAGDLDCSNCHLVREMMHANASRTIYFLVHATGVGSFQHAIVDRRYTATGAEGLHFPGRQLLYFDLTNHTIESASDFIASNVEKLKNDTTGHHFLDTGYNFSGAVRTDMANSHTAMEMNMLHTIVSAPFENVTTDAASPPAAQFIGAPPAAELPAPVPAPAPPAAHEQNAVVATLLFKVEEFYAAANSRPAAKRADVKILESSQVTQQAGGSSAATATMYPSMVDRKRKRAQATPSRMAPHEVIQYLRATAVETDKELETLNNFFKVCDGEDKALITFSVEQIRSIKKKIGRIINKPVTAMSSRRMARFIDEIDTIKEEKARVFEEIIKILKNPRRKRENDGSSGSNRKNVGGSSGGKKKTVGGSSGSKKKNVGRPSAKKAQK

SEQ ID NO: 39 - protein, 256 aa, Arabidopsis thaliana
  REGION 1..256: note = AtRKD4 (AT5G53040)
MSSSKHSSVFNYSALFLSLFLQQMDQNSLHHLDSPKIENEYEPDSLYDMLDKLPPLDSLLDMEDLKPNAGLHFQFHYNSFEDFFENIEVDNTIPSDIHLLTQEPYFSSDSSSSSPLAIQNDGLISNVKVEKVTVKKKRNLKKKRQDKLEMSEIKQFFDRPIMKAAKELNVGLTVLKKRCRELGIYRWPHRKLKSLNSLIKNLKNVGMEEEVKNLEEHRFLIEQEPDAELSDGTKKLRQACFKANYKRRKSLGDDYY

SEQ ID NO: 40 - protein, 16 aa, synthetic construct
  REGION 1..16: note = Synthetic peptide of TaRKD1-7D
CFKENYKRRRAAASVN

SEQ ID NO: 41 - protein, 15 aa, synthetic construct
  REGION 1..15: note = Synthetic peptide of TaRKD1-7D
ARWPHRKMKSLRSLI

SEQ ID NO: 42 - DNA, 1104 bp, synthetic construct
  source 1..1104: mol_type = other DNA
  misc_feature 1..1104: note = synTaRKD1 - codon optimised sequence of TaRKD1
atggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttag

SEQ ID NO: 43 - DNA, 1323 bp, synthetic construct
  source 1..1323: mol_type = other DNA
  misc_feature 1..1323: note = synOsRKD3: codon optimized sequence of OsRKD3
atggaaatgcacgagtgctgctactacgggggttccatcggaggcgactggcttaatccgctcgcggcaataccgccgccgtgttcatcatccagctcatcgtggtctagccagctgctgctgctctcggaccatgacgatgtgttgctccacagcgccggcgatcacggaggggcggtggcgggaattggcggtgcctgtatgactgctgatctggtggtgagggacgaggagatggagatggccgccggctacctccctgtcgctgcgtccgcagccgcggcggcggatgtcgatcactacatgtaccagcagttccagcttgagcccgatcagttcgtgtcaaccttgccggctgttgccgttgccgtggccgcgacagcagggggaggatcacacgacgacgaactcctcaggatgccattcaccgacatcgatttggatgcatttgccgatgccagagatgtggtcgtcggagttggggagccgaagccctctccccagcatacccttgacgctgcaatcgcgcttcctgcagtaggcggcggcggcgctcatcatttcggaacgcaagacgatgacgtgaagttcgacgtaaccaaacagcggaacgatgctgcgctcgctggcgatgatagtctgagtatggtgatcgtggagtcgtacgagatgggaatgagaagacatgcggctgagcaagagcaggaacagaagccaaagatcattacctcagcagccactaccctcacgccgctcccgctgccgcctcctcctccaccaccacctagagtgacgcggtcaaggagagatggtagctcagcagcaaccgccgggggaaaaacgcgccttgaccacataggattcgaggatcttagaagatatttttacatgcccatcacaaaggcagcgcgcgagatgaatgtggggcttacagtcttgaagaagaggtgcagagaactgggcgttgcgcggtggccacaccgcaaaatgaagagcttgaaaagcttgatccttaatgaaatggggagcaagggtatgtctgctgccgcgatgaggcgcgagctggaagcccttgaaaactgctgcgcactgatggagaggaaccccgcggtggaacttacagaacggactaagaagttgcgccaggcctgcttcaaggaaaattataagcgcaggcgcgcggctgctgtcgacgtgctggatctcgaccactgcttttctttcgcggctggtcactgccacagacaccaccaccaacagcttgcgctgcccccaccacctgcggcggctgctgatcaccgtcgtcgcgattttttcggctac

SEQ ID NO: 44 - DNA, 768 bp, synthetic construct
  source 1..768: mol_type = other DNA
  misc_feature 1..768: note = synAtRKD4: codon optimized sequence of AtRKD4
atgtcctcatcgaagcactcatctgttttcaattattctgccttatttcttagtctgttcttgcagcaaatggatcaaaatagcttacaccacctggactcaccgaagattgagaatgagtacgaaccggactccttatatgacatgttagataagctgcctcctctggactcacttttagacatggaagacctgaagccaaacgccggacttcacttccaattccactataacagctttgaagacttttttgagaatatcgaagtagacaataccattcctagtgacatccacttactgacacaggaaccgtacttttcgagtgattccagcagttcgtccccgctggccattcaaaatgacggcttaatctccaatgtaaaagtggaaaaggtcaccgtgaaaaagaagcgtaatctgaaaaaaaaacgccaagataagttagaaatgtcggagatcaagcagttcttcgaccgccccattatgaaggcggcgaaggagctgaatgtgggtttaactgtattaaaaaagcgctgtcgtgagttgggcatctatcgctggcctcaccgtaaacttaagtctcttaactccctgattaaaaatttgaagaacgtggggatggaggaagaggtaaagaatttggaggagcaccgcttccttattgaacaggagccggatgctgagctttctgatgggaccaagaaactgcgtcaagcctgcttcaaagctaattacaagcgccgcaagtccttgggggatgattactac